(19) United States
(12) Patent Application Publication
(10) Pub. No.: US 2026/0027131 A1
(43) Pub. Date: Jan. 29, 2026

(54) PRODRUGS, PRODRUG COMPOSITIONS AND RELATED METHODS

(71) Applicant: Praesidia Biotherapeutics Inc., Arlington, MA (US)

(72) Inventors: Pradip Kumar Majumder, Chestnut Hill, MA (US); Ahmed Masud Aman, Oakville, Ontario (CA); Pravin Kumar Dakshinamurthy, Bangalore (IN); Salma Shirin, Oakville, Ontario (CA); Sohang Chatterjee, Lexington, MA (US)

(21) Appl. No.: 18/996,739

(22) PCT Filed: Jul. 18, 2023
(86) PCT No.: PCT/US2023/070399

§ 371 Date: Jan. 17, 2025

Related U.S. Application Data
(60) Provisional application No. 63/390,510, filed on Jul. 19, 2022.

(51) Int. Cl. A61K 31/573; A61K 47/54; A61K 47/64; C07J 41/00

(52) U.S. Cl. CPC A61K 31/573; A61K 47/545; A61K 47/64; C07J 41/005

(57) ABSTRACT

Prodrugs including cleavable moieties capable of specific binding to a target are presented. Related pharmaceutical compositions and methods are also presented.

### CROSS REFERENCE TO RELATED APPLICATION

This application claims a benefit of, and priority to, U.S. Provisional Patent Application No. 63/390,510, filed Jul. 19, 2022, the disclosure of which is incorporated by reference in its entirety.

### TECHNICAL FIELD

Embodiments of the present invention generally relate to prodrugs, and more particularly to prodrugs including cleavable moieties capable of specific binding to a target.

### BACKGROUND

The development of prodrugs has become one of the strategies to improve the physicochemical, pharmacokinetic, and/or pharmacodynamic properties of pharmaceutically active agents, and thereby enhance their efficacy and/or reduce their side effects. For example, prodrugs provide possibilities to overcome various barriers to drug formulation and delivery such as poor aqueous solubility, physical and/or chemical instability, insufficient absorption, rapid pre-systemic metabolism, inadequate brain penetration, toxicity, and/or local irritation. Prodrugs can also improve drug targeting and duration of action. Prodrugs are typically inactive derivatives of a drug molecule that require chemical or enzymatic biotransformation to release the active parent drug in the body. Therefore, prodrugs need to be efficiently converted to the parent drugs to reach pronounced efficacy as soon as the drug target has been reached.

Thus, there is a need for new prodrug chemistries that provide for specific targeting of the drug to target cells and/or target sites while minimizing toxicity.

### SUMMARY

The following summary is illustrative only and is not intended to be in any way limiting. In addition to the illustrative aspects, example embodiments, and features described, further aspects, example embodiments, and features will become apparent by reference to the following detailed description.

In some aspects of the present invention, a compound having a structure of formula (I), or a pharmaceutically acceptable salt thereof is presented. Formula (I) is:

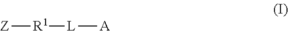

wherein Z is a therapeutic moiety;
L is a linker moiety bonded to Z via R¹, wherein R¹ is O, N, or NH, and L is selected from:

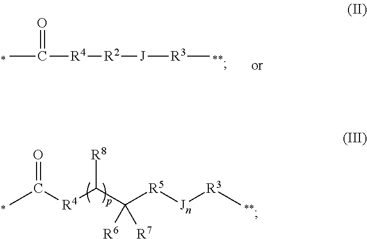

wherein * denotes the point of attachment of the linker moiety to R¹ and ** denotes the point of attachment of the linker moiety to A;
J is an aryl group or a heteroaryl group optionally substituted with one or more functional groups;
R² and R³ are independently at each occurrence a bond, a C₁-C₆ alkylene group, or a C₁-C₆ alkenylene group;
R⁴ is a bond, O or NR′;
R⁵ is a bond, a C₁-C₆ alkylene group, O or NR′, wherein R′ is independently hydrogen or a C₁-C₃ alkyl group;
R⁶, R⁷, and R⁸ are independently at each occurrence hydrogen or a C₁-C₃ alkyl group, with the proviso that at least one R⁶, R⁷, or R⁸ is a C₁-C₃ alkyl group or R⁶ and R⁷ together with a carbon to which each is attached form a C₃-C₆ cycloalkyl group;
“n” is an integer from 0 to 1, “p” is an integer from 0 to 1; and
A is an amino acid moiety or a peptide moiety.

In some aspects of the present invention, compounds having a structure of formula (IX) to (XIII), or pharmaceutically acceptable salt thereof, are presented. Formula (IX) to (XIII) is

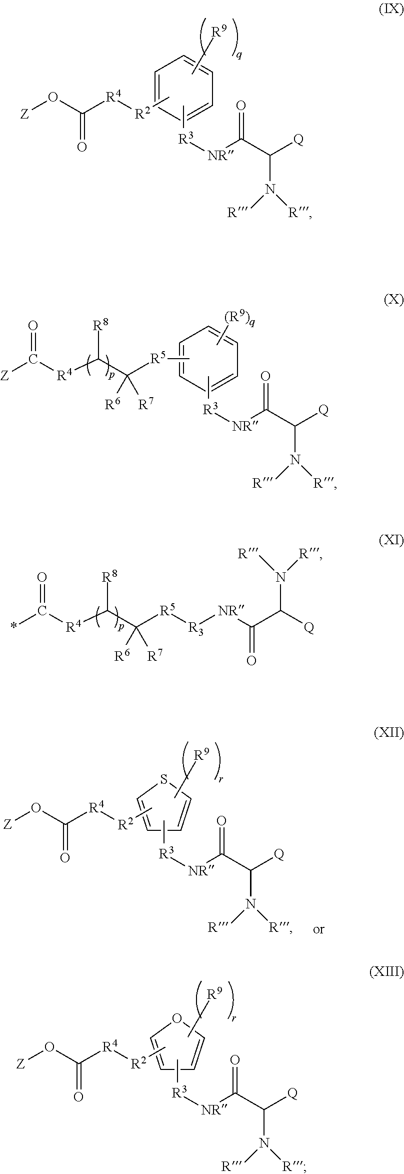

wherein Z is a therapeutic moiety;
“p” is an integer from 0 to 1; “q” is an integer from 0 to 4, “r” is an integer from 0 to 2; R² and R³ are independently at each occurrence a bond, a C₁-C₆ alkylene group, or a C₁-C₆ alkenylene group;
R⁴ is a bond, O or NR′;
R⁵ is a bond, a C₁-C₆ alkylene group, O or NR′, wherein R′ is independently hydrogen or a C₁-C₃ alkyl group;
R⁶, R⁷, and R⁸ are independently at each occurrence hydrogen or a C₁-C₃ alkyl group, with the proviso that at least one R⁶, R⁷, or R⁸ is a C₁-C₃ alkyl group or R⁶ and R⁷ together with a carbon to which each is attached form a C₃-C₆ cycloalkyl group;
R⁹ is independently at each occurrence a functional group selected from alkoxy, hydroxy, halogen, amine, nitro, cyano, or dialkylamine;
R″ is hydrogen or a C₁-C₃ alkyl group; and
R′″ is independently at each occurrence hydrogen, a C₁-C₃ alkyl group, or a C(═O)CH(Q)NR′″ moiety, Q is an amino acid side chain.

In some aspects of the present invention, a pharmaceutical composition is presented. The pharmaceutical composition includes a compound of formula (I), (IX), (X), (XI), (XXII), or (XIII), or a pharmaceutically acceptable salt, solvate, hydrate, polymorph, or co-crystal thereof, and a pharmaceutically carrier, diluent, or excipient.

In some aspects of the present invention, a process for preparing a pharmaceutical composition in a solid or a liquid formulation is presented. The process includes mixing a structure of formula (I), (IX), (X), (XI), (XXII), or (XIII), or a pharmaceutically acceptable salt, solvate, hydrate, polymorph, or co-crystal thereof with a pharmaceutically acceptable carrier, diluent, or excipient.

In some aspects of the present invention, a method of treating an inflammation is presented. The method includes administering to a patient an effective amount of a pharmaceutical compositing including a compound having a structure of formula (I), (IX), (X), (XI), (XXII) or (XIII), or a pharmaceutically acceptable salt, solvate, hydrate, polymorph, or co-crystal thereof.

In some aspects of the present invention, a method of treating a chronic respiratory disease is presented. The method includes administering to a patient an effective amount of a pharmaceutical composition including a compound having structure of formula formula (I), (IX), (X), (XI), (XXII) or (XIII), or a pharmaceutically acceptable salt, solvate, hydrate, polymorph, or co-crystal thereof. In some embodiments. In some embodiments, the chronic respiratory disease is chronic obstructive pulmonary disease (COPD), sarcoidosis, or asthma.

In some embodiments, a method of treating edema is presented. The method includes administering to a patient an effective amount of a pharmaceutical composition including a compound having a structure of formula (I), (IX), (X), (XI), (XXII) or (XIII), or a pharmaceutically acceptable salt, solvate, hydrate, polymorph, or co-crystal thereof. In some embodiments, the edema is cerebral edema, pulmonary edema, or peripheral edema.

In some aspects of the present invention, a method of treating a brain disease is presented. The method includes administering to a patient an effective amount of a pharmaceutical composition including a compound having a structure of formula (I), (IX), (X), (XI), (XXII) or (XIII), or a pharmaceutically acceptable salt, solvate, hydrate, polymorph, or co-crystal thereof. In some embodiments, the brain disease includes glioblastoma, medulloblastoma, glioma, or a brain metastatic disease.

### Definitions

The terminology used herein is for the purpose of describing particular example embodiments only and is not intended to be limiting. Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

To more clearly and concisely describe and point out the subject matter of the claimed invention, the following definitions are provided for specific terms that are used in the following description and the claims appended hereto. Definitions of specific functional groups and chemical terms are described in more detail below. For purposes of this invention, the chemical elements are identified in accordance with the Periodic Table of the Elements, CAS version, Handbook of Chemistry and Physics, 75th Ed., inside cover, and specific functional groups are generally defined as described therein. Unless otherwise stated, structures depicted herein are also meant to include compounds that differ only in the presence of one or more isotopically enriched atoms. For example, compounds having the present structures including the replacement of hydrogen by deuterium or tritium, the replacement of a carbon by a 13C- or 14C-enriched carbon, or the replacement of a fluorine by 18F-enriched fluorine, are within the scope of this invention. Such compounds are useful, for example, as analytical tools, as probes in biological assays, or as therapeutic agents in accordance with the present invention.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms.

Where a particular enantiomer is preferred, it may, in some embodiments be provided substantially free of the corresponding enantiomer, and may also be referred to as “optically enriched.” “Optically-enriched,” as used herein, means that the compound is made up of a significantly greater proportion of one enantiomer. In certain embodiments, the compound is made up of at least about 90% by weight of a preferred enantiomer. In other embodiments, the compound is made up of at least about 95%, 98%, or 99% by weight of a preferred enantiomer. Preferred enantiomers may be isolated from racemic mixtures by any method known to those skilled in the art, including chiral high-pressure liquid chromatography (HPLC) and the formation and crystallization of chiral salts or prepared by asymmetric syntheses.

As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated moieties, groups, compounds, steps, elements, and/or components, but do not preclude the presence or addition of one or more other moieties, groups, compounds, steps, elements, components, and/or groups thereof.

As used herein, the term “alkyl group” refers to a saturated monovalent group consisting of a linear or branched array of atoms that is not cyclic. Alkyl groups are defined to include at least one carbon atom and are represented by formula CₙH₂ₙ₊₁. The array of atoms included in the alkyl group may be composed exclusively of carbon and hydrogen. By way of example, the term “C₁-C₁₀ alkyl group” contains at least one but no more than 10 carbon atoms. A methyl group (i.e. CH₃—) is an example of a monovalent C₁ alkyl group. A decyl group (i.e., CH₃(CH₂)₉—) is an example of a monovalent C₁₀ alkyl group. Non-limiting examples of alkyl groups include methyl, ethyl, n-propyl, isopropyl, n-butyl, t-butyl, isobutyl, sec-butyl, n-pentyl, n-hexyl, n-heptyl, n-octyl, and the like.

As used herein, the term “alkylene group” refers to a saturated divalent group consisting of a linear or branched array of atoms that is not cyclic. Alkylene groups are defined to include at least one carbon atom and are represented by formula CₙH₂ₙ. The array of atoms included in the alkylene group may be composed exclusively of carbon and hydrogen. By way of example, the term “C₁-C₁₀ alkylene group” contains at least one but no more than 10 carbon atoms. The group —CH₂CH₂— is an example of a divalent linear C₂ alkylene group. Similarly, the group —CH(CH₃)CH₂— is an example of a divalent branched C₃ alkylene group. Non-limiting examples of alkylene groups include methylene ethylene, propylene, 2-methylpropylene, pentylene, hexylene and the like.

As used herein, the term “alkenylene group” refers to an unsaturated divalent group consisting of a linear or branched array of atoms that is not cyclic. Alkenylene groups are defined to include at least one carbon atom and are represented by formula CₙH₂ₙ₋₂. The array of atoms included in the alkenylene group may be composed exclusively of carbon and hydrogen. By way of example, the term “C₁-C₁₀ alkenylene group” contains at least one but no more than 10 carbon atoms. The group-CH═CH— is an example of a divalent linear C₂ alkenylene group. Similarly, the group CH(CH₃)═CH—) is an example of a divalent branched C₃ alkylene group.

As used herein the term “cycloalkyl group” refers to a group having a valence of one, and consisting of an array of atoms that is cyclic but which is not aromatic. A “cycloalkyl” may include one or more noncyclic components. For example, a cyclohexylmethyl group (C₆H₁₁CH₂—) is a cycloalkyl group that includes a cyclohexyl ring (the array of atoms that is cyclic but which is not aromatic) and a methylene group (the noncyclic component). By way of example, the term “a C₃-C₁₀ cycloalkyl group” includes cycloalkyl groups containing at least three but no more than 10 carbon atoms. The cyclohexylmethyl group (C₆H₁₁CH₂—) represents a C₇ cycloalkyl group.

As used herein the term “aryl group” refers to a monovalent aromatic hydrocarbon radical derived by the removal of one hydrogen atom from a single carbon atom of a parent aromatic ring system. Aryl group encompasses 5- and 6-membered carbocyclic aromatic rings, for example, benzene; bicyclic ring systems wherein at least one ring is carbocyclic and aromatic, for example, naphthalene, indane, and tetralin; and tricyclic ring systems wherein at least one ring is carbocyclic and aromatic, for example, fluorene.

The term “aryl group” also encompasses multiple ring systems having at least one carbocyclic aromatic ring fused to at least one carbocyclic aromatic ring, cycloalkyl ring, or heterocycloalkyl ring. For example, aryl group includes 5- and 6-membered carbocyclic aromatic rings fused to a 5- to 7-membered heterocycloalkyl ring containing one or more heteroatoms chosen from N, O, and S. For such fused, bicyclic ring systems wherein only one of the rings is a carbocyclic aromatic ring, the point of attachment may be at the carbocyclic aromatic ring or the heterocycloalkyl ring. Examples of aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In certain embodiments, an aryl group can include from 5 to 20 carbon atoms, and in certain embodiments, from 5 to 12 carbon atoms.

As used herein, the term “heteroaryl group” refers to a monovalent heteroaromatic radical derived by the removal of one hydrogen atom from a single atom of a parent heteroaromatic ring system. Heteroaryl group encompasses multiple ring systems having at least one aromatic ring fused to at least one other ring, which can be aromatic or non-aromatic in which at least one ring atom is a heteroatom. Heteroaryl group encompasses 5- to 12-membered aromatic, monocyclic rings (such as 5- to 7-membered rings) containing one or more, for example, from 1 to 4, or in certain embodiments, from 1 to 3, heteroatoms chosen from N, O, and S, with the remaining ring atoms being carbon; and bicyclic heterocycloalkyl rings containing one or more, for example, from 1 to 4, or in certain embodiments, from 1 to 3, heteroatoms chosen from N, O, and S, with the remaining ring atoms being carbon and wherein at least one heteroatom is present in an aromatic ring. For example, heteroaryl includes a 5- to 7-membered heteroaromatic ring fused to a 5- to 7-membered cycloalkyl ring. For such fused, bicyclic heteroaryl ring systems wherein only one of the rings contains one or more heteroatoms, the point of attachment may be at the heteroaromatic ring or the cycloalkyl ring. In certain embodiments, when the total number of N, S, and O atoms in the heteroaryl group exceeds one, the heteroatoms are not adjacent to one another. In certain embodiments, the total number of N, S, and O atoms in the heteroaryl group is not more than two. In certain embodiments, the total number of N, S, and O atoms in the aromatic heterocycle is not more than one.

Examples of heteroaryl groups include, but are not limited to, groups derived from acridine, arsindole, carbazole, beta-carboline, chromane, chromene, cinnoline, furan, furazan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

As used herein, the term “amino acid” refers to both naturally occurring and non-naturally occurring amino acids. Therefore, the term “amino acid” includes naturally occurring proteogenic L-amino acids; D-amino acids; chemically modified amino acids such as amino acid variants and derivatives; naturally occurring non-proteogenic amino acids such as norleucine, β-alanine, ornithine, etc.; and chemically synthesized compounds having properties known in the art to be characteristic of amino acids.

Some of the standard naturally occurring nonpolar (hydrophobic) amino acids include alanine (Ala), leucine (Leu), isoleucine (Ile), valine (Val), proline (Pro), phenylalanine (Phe), tryptophan (Trp) and methionine (Met). The polar neutral amino acids include glycine (Gly), serine (Ser), threonine (Thr), cysteine (Cys), tyrosine (Tyr), asparagine (Asn), and glutamine (Gln). The positively charged (basic) amino acids include arginine (Arg), lysine (Lys), and histidine (His). The negatively charged (acidic) amino acids include aspartic acid (Asp) and glutamic acid (Glu). The non-standard amino acids may be formed, for example, in the body by posttranslational modification. Some examples of such naturally occurring non-standard amino acids include selenocysteine and pyrolysine. Other non-limiting examples of amino acids include α-methyl amino acids (e.g., α-methyl alanine), β-amino acids, histidine-like amino acids (e.g., 2-amino-histidine, β-hydroxy-histidine, homohistidine, α-fluoromethyl-histidine, and α-methyl-histidine), amino acids having an extra methylene in the side chain (“homo” amino acids) and amino acids in which a carboxylic acid functional group in the side chain is replaced with a sulfonic acid group (e.g., cysteic acid).

The term “amino acid” as used herein encompasses amino acids wherein one or more amino groups in the amino acid compounds may be further substituted with an alkyl group to form a monoalkyl amino acid or a dialkyl amino acid. For example, N,N-dimethyl-L-phenylalanine is an example of a dialkyl amino acid and methyl-L-alanine is an example of a monoalkyl amino acid. Further, in the present description, the designation of an amino acid without specifying its stereochemistry is intended to encompass either the L or D form of the amino acid or a racemic mixture thereof.

As used herein, the term “amino acid side chain” refers to the moiety attached to the α-carbon of an amino acid, as defined herein. For example, amino acid side chains from naturally-occurring amino acids include hydrogen (as in glycine), —CH₃ (as in alanine), —CH(CH₃)₂ (as in valine), —CH₂CH(CH₃)₂ (as in leucine), —CH(CH₃)CH₂CH₃ (as in isoleucine), benzyl (as in phenylalanine), p-hydroxybenzyl (as in tyrosine), —CH₂-(1H-indol-3-yl) (as in tryptophan), —CH₂-(1H-imidazol-5-yl) (as in histidine), —CH₂OH (as in serine), —CH(OH)CH₃ (as in threonine), —CH₂SH (as in cysteine), —CH₂CH₂SCH₃ (as in methionine), —CH₂COOH (as in aspartic acid), —CH₂CH₂COOH (as in glutamic acid), —CH₂CONH₂ (as in asparagine), —CH₂CH₂CONH₂ (as in glutamine), —CH₂CH₂CH₂CH₂NH₂ (as in lysine), and —CH₂CH₂CH₂NHC(NH)(NH₂) (as in arginine).

As used herein, the term “peptide” refers to a linear sequence of two or more amino acids connected to each other by peptide bonds between the alpha-amino and carboxyl groups of adjacent amino acids. The amino acids may be naturally occurring amino acids or some other non-naturally occurring amino acids, as defined hereinabove. The peptides may be of a variety of lengths, either in their neutral (uncharged) form or in forms such as their salts. The peptides may be either free of modifications such as glycosylations, side chain oxidation, or phosphorylation or comprising such modifications. Substitutes for an amino acid within the sequence may also be selected from other members of the class to which the amino acid belongs. A suitable peptide may also include peptides modified by additional substituents attached to the amino side chains, such as glycosyl units, lipids, or inorganic ions such as phosphates as well as chemical modifications of the chains. Thus, the term “peptide” or its equivalent may be intended to include the appropriate amino acid sequence referenced, subject to the foregoing modifications, which do not destroy its functionality.

The terms “amino acid moiety” or “peptide moiety” as used herein refer to a residue of an amino acid or a peptide after binding to the linker, for example, via an amide or an ester bond.

As used herein the term “cleavable moiety” refers to a moiety having a bond that is cleavable under specified conditions of use, for example, following administration to a patient. The bond may be cleaved by enzymatic or non-enzymatic means. The cleavage may proceed spontaneously, such as via a hydrolysis reaction, or it may be catalyzed or induced by another agent, such as an enzyme, an acid, a base, or a change of or exposure to a physical or environmental parameter, such as temperature, pH, etc. The agent may be endogenous to the conditions of use, such as an enzyme present in the systemic circulation of a patient to which the prodrug is administered or the acidic conditions of the stomach, or the agent may be supplied exogenously. As used herein the term “enzymatically cleavable moiety” refers to a moiety having a bond that is cleavable by enzymatic means. In certain embodiments, the cleavable moiety has a bond that is cleavable by enzymatic means present in the diseased organ, tissue, or cells.

As used herein, the term “specific binding” refers to the specific recognition of one of two different molecules for the other compared to substantially less recognition of other molecules. The molecules may have areas on their surfaces or in cavities giving rise to specific recognition between the two molecules arising from one or more of electrostatic interactions, hydrogen bonding, or hydrophobic interactions. Specific binding examples include, but are not limited to, antibody-antigen interactions, enzyme-substrate interactions, polynucleotide interactions, receptor interactions, and the like. In some embodiments, specific binding refers to the binding of the compounds of the present invention to one or more enzymes located within or in the proximity of the target cells or the target site (such as a target tissue or organ).

As used herein, the term “target,” refers to moieties that are naturally more expressed in a diseased state versus a healthy state, or alternatively, to moieties that are naturally more expressed in the target cells or target site (such as a tissue or an organ). In general, the compounds of the present invention may bind to a target through one or more discrete chemical moieties of the target or a three-dimensional structural component of the target (e.g., 3D structures resulting from peptide folding). The target may include one or more of natural or modified peptides, proteins (e.g., antibodies, affibodies, aptamers, or lectins), nucleic acids (e.g., polynucleotides, DNA, RNA, or aptamers); polysaccharides (e.g., sugars), lipids, enzymes, enzyme substrates, ligands, receptors, antigens, or haptens. In some embodiments, targets may include one or more enzymes present within or in proximity to the target cells or the target site (such as a target tissue or organ). Non-limiting examples of targets include peptidases, brain amidases, macrophage mannose receptors, focal adhesion kinases, and the like.

As used herein, the term “prodrug” refers to a derivative of a drug or a pharmaceutically active agent that is administered in an inactive or less than fully active form and is then converted to its active form within the body. In some embodiments, the transformation releases the parent drug or pharmaceutically active agent. In some embodiments, a bioactive derivative of the parent drug or pharmaceutically active agent is generated.

As used herein, the term “therapeutically effective amount” refers to an amount (of a compound) that is sufficient to provide a therapeutic benefit to a patient in the treatment or management of a disease or disorder, or to delay or minimize one or more symptoms associated with the disease or disorder.

As used herein, the term “pharmaceutically acceptable salt” refers to any salt suitable for administration to a patient. Examples of salts include, but are not limited to, acid-derived, base-derived, organic, inorganic, amine, and alkali or alkaline earth metal salts, including but not limited to calcium salts, magnesium salts, potassium salts, sodium salts, salts of hydrochloric acid, hydrobromic acid, sulfuric acid, nitric acid, phosphoric acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p toluenesulfonic acid, salicylic acid, and the like.

### DETAILED DESCRIPTION

In some embodiments, a compound having a formula (I), or a pharmaceutically acceptable salt thereof, is presented.

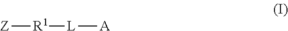

wherein Z is a therapeutic moiety;
L is a linker moiety bonded to Z via R¹, wherein R¹ is O, N, or NH, and L is selected from:

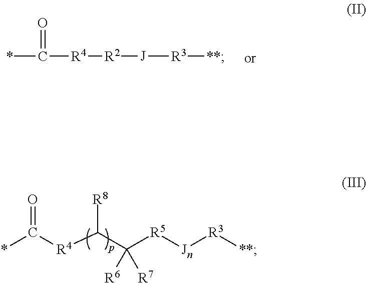

wherein * denotes the point of attachment of the linker moiety to R¹ and ** denotes the point of attachment of the linker moiety to A;
J is an aryl group or a heteroaryl group optionally substituted with one or more functional groups;
R² and R³ are independently at each occurrence a bond, a C₁-C₆ alkylene group, or a C₁-C₆ alkenylene group;
R⁴ is a bond, O or NR′;
R⁵ is a bond, a C₁-C₆ alkylene group, O or NR′, wherein R′ is independently hydrogen or a C₁-C₃ alkyl group;
R⁶, R⁷, and R⁸ are independently at each occurrence hydrogen or a C₁-C₃ alkyl group, with the proviso that at least one R⁶, R⁷, or R⁸ is a C₁-C₃ alkyl group or R⁶ and R⁷ together with a carbon to which each is attached form a C₃-C₆ cycloalkyl group;
“n” is an integer from 0 to 1, “p” is an integer from 0 to 1; and
A is an amino acid moiety or a peptide moiety.

In some embodiments, A is an enzymatically cleavable moiety. In certain embodiments, A is an amino acid moiety or a peptide moiety bonded to the linker moiety via an amide group. In certain embodiments, A includes a residue of one or more amino acids selected from the group consisting of glycine, valine, isoleucine, proline, phenyl alanine, tryptophan, and any combinations or modifications thereof. In some embodiments, one or more amino groups in the amino acid moiety or the peptide moiety may be further substituted with a C₁-C₃ alkyl group.

The linker moiety “L” may be selected from one of the following structures:

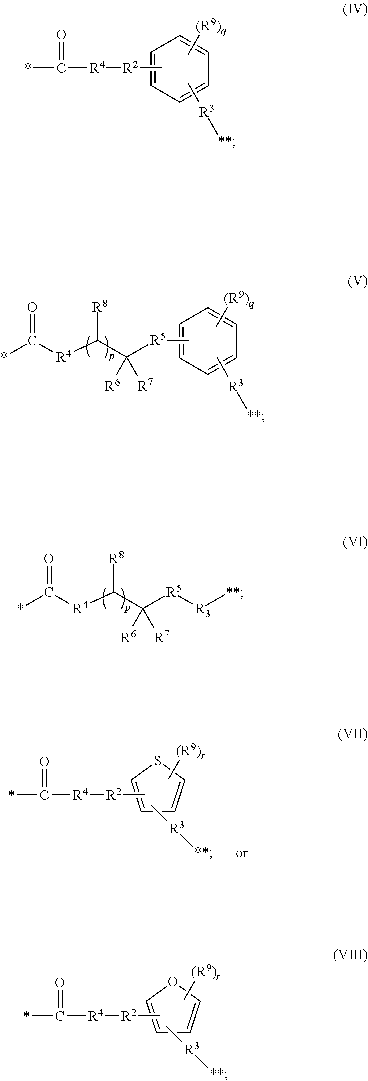

wherein ‘n”, “p” and R¹ to R⁸ are as defined hereinabove, “q” is an integer from 0 to 4, “r” is an integer from 0 to 2; and R⁹ is a functional group selected from alkoxy, hydroxy, halogen, amine, nitro, cyano, or dialkylamine

The linker moiety L is capable of undergoing an intramolecular cleavage to release the therapeutic moiety. In some embodiments, the linker moiety is capable of undergoing an intramolecular cyclization reaction to release the therapeutic moiety. In some embodiments, the linker moiety L is capable of undergoing an intramolecular cyclization reaction based on the Thorpe-Ingold mechanism to release the therapeutic moiety. In some embodiments, the linker moiety L may undergo an intramolecular cyclization reaction to release the therapeutic moiety, a lactam moiety, and the amino acid moiety or the peptide moiety.

In some embodiments compounds having formula (IX) to (XIII), or pharmaceutically acceptable salts thereof, are presented.

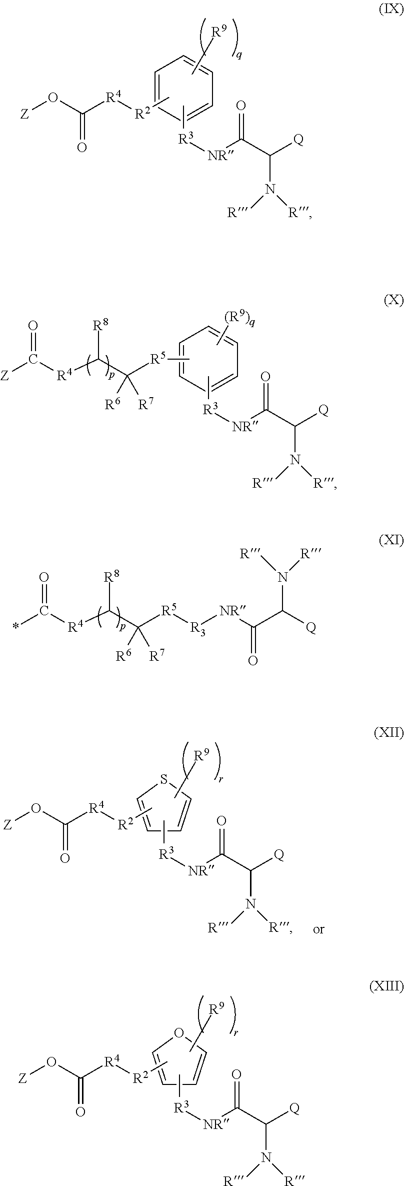

wherein Z is a therapeutic moiety;
“p” is an integer from 0 to 1; “q” is an integer from 0 to 4, “r” is an integer from 0 to 2;
R² and R³ are independently at each occurrence a bond, a C₁-C₆ alkylene group, or a C₁-C₆ alkenylene group;
R⁴ is a bond, O or NR′;
R⁵ is a bond, a C₁-C₆ alkylene group, O or NR′, wherein R′ is independently hydrogen or a C₁-C₃ alkyl group;
R⁶, R⁷, and R⁸ are independently at each occurrence hydrogen or a C₁-C₃ alkyl group, with the proviso that at least one R⁶, R⁷, or R⁸ is a C₁-C₃ alkyl group or R⁶ and R⁷ together with a carbon to which each is attached form a C₃-C₆ cycloalkyl group;
R⁹ is independently at each occurrence a functional group selected from alkoxy, hydroxy, halogen, amine, nitro, cyano, or dialkylamine amino;
R″ is hydrogen or a C₁-C₃ alkyl group; and
R′″ is independently at each occurrence hydrogen, a C₁-C₃ alkyl group, or a C(═O)CH(Q)NR′″ moiety, Q is an amino acid side chain.

In some embodiments, compounds having a formula (IX) to (XIII) or pharmaceutically acceptable salts thereof are presented, wherein R², R³, and R⁵ are independently a bond or a C₁-C₃ alkylene group, R⁴ is a bond, O, or NH, R⁶, and R⁷ are independently at each occurrence a methyl group, or R⁶ and R⁷ together with a carbon to which each is attached form a cyclopropyl group, and “p” is 0 or R⁸ is a C₁-C₃ alkyl group.

In some embodiments, compounds having a formula (IX) to (XIII) or pharmaceutically acceptable salts thereof are presented, wherein R², R³, and R⁵ are independently a bond or a C₁-C₃ alkylene group, R⁴ is a bond, R⁶ and R⁷ are independently at each occurrence a methyl group, or R⁶ and R⁷ together with a carbon to which each is attached form a cyclopropyl group, and “p” is 0 or R⁸ is a C₁-C₃ alkyl group.

In some embodiments, compounds having a formula (IX) to (XIII) or pharmaceutically acceptable salts thereof are presented, wherein R², R³, R⁴, and R⁵ are independently a bond, R⁶ and R⁷ are independently at each occurrence a methyl group, or R⁶ and R⁷ together with a carbon to which each is attached form a cyclopropyl group, and “p” is 0 or R⁸ is a methyl group.

In some embodiments, compounds having a formula (IX) to (XIII) or pharmaceutically acceptable salts thereof are presented, wherein R², R³, and R⁵ are independently a bond or a C₁-C₃ alkylene group, R⁴ is a bond, R⁶ and R⁷ are independently at each occurrence a methyl group, or R⁶ and R⁷ together with a carbon to which each is attached form a cyclopropyl group, “p” is 0 or R⁸ is a C₁-C₃ alkyl group, “q” is 1-2, “r” is 1-2, and R⁹ is independently at each occurrence alkoxy, halo, or a dialkylamine.

In some embodiments, compounds having a formula (IX) to (XIII) or pharmaceutically acceptable salts thereof are presented, wherein R², R³, R⁴, and R⁵ are independently a bond, R⁶, and R⁷ are independently at each occurrence a methyl group, or R⁶ and R⁷ together with a carbon to which each is attached form a cyclopropyl group, “p” is 0 or R⁸ is a methyl group, and Q includes valine, proline, phenylalanine, or tryptophan side chain.

In some embodiments, compounds having a formula (IX) to (XIII) or pharmaceutically acceptable salts thereof are presented, wherein R², R³ and R⁵ are independently a bond or a C₁-C₃ alkylene group, R⁴ is a bond, R⁶ and R⁷ are independently at each occurrence a methyl group, or R⁶ and R⁷ together with a carbon to which each is attached form a cyclopropyl group, “p” is 0 or R⁸ is a C₁-C₃ alkyl group, “q” is 1-2, “r” is 1-2, R⁹ is independently at each occurrence alkoxy, halo, or a dialkylamine, and Q is a valine, proline, phenylalanine, or tryptophan side chain.

In some embodiments, compounds having a formula (IX) to (XIII) or pharmaceutically acceptable salts thereof are presented, wherein R², R³, R⁴, and R⁵ are independently a bond, R⁶ and R⁷ are independently at each occurrence a methyl group, or R⁶ and R⁷ together with a carbon to which each is attached form a cyclopropyl group, and “p” is 0 or R⁸ is a methyl group, “p” is 0 or R⁸ is a C₁-C₃ alkyl group, “q” is 1-2, “r” is 1-2, and R⁹ is independently at each occurrence alkoxy, halo, or a dialkylamine.

In some embodiments, compounds having a formula (IX) to (XIII) or pharmaceutically acceptable salts thereof are presented, wherein R², R³, and R⁵ are independently a bond or a C₁-C₃ alkylene group, R⁴ is a bond, R⁶ and R⁷ are independently at each occurrence a methyl group, or R⁶ and R⁷ together with a carbon to which each is attached form a cyclopropyl group, “p” is 0 or R⁸ is a C₁-C₃ alkyl group, “q” is 1-2, “r” is 1-2, and R⁹ is independently at each occurrence alkoxy, halo, or a dialkylamine.

As noted earlier, Z is a therapeutic moiety obtained from a therapeutic agent. Non-limiting examples of therapeutic agents include ACE-inhibitors; anti-anginal drugs; anti-arrhythmias; anti-asthmatics; anti-cholesterolemics; anti-convulsants; anti-depressants; anti-diarrhea preparations; anti-histamines; antihypertensive drugs; anti-infectives; anti-inflammatory agents; anti-lipid agents; anti-manics; anti-nauseants; antistroke agents; anti-thyroid preparations; anti-tumor drugs; anti-tussives; anti-uricemic drugs; anti-viral agents; acne drugs; alkaloids; amino acid preparations; anabolic drugs; analgesics; anesthetics; angiogenesis inhibitors; antacids; anti-arthritics; antibiotics; anticoagulants; antiemetics; antiobesity drugs; antiparasitics; antipsychotics; antipyretics; antispasmodics; antithrombotic drugs; anxiolytic agents; appetite stimulants; appetite suppressants; beta blocking agents; bronchodilators; cardiovascular agents; cerebral dilators; chelating agents; cholecystokinin antagonists; chemotherapeutic agents; cognition activators; contraceptives; coronary dilators; cough suppressants; decongestants; deodorants; dermatological agents; diabetes agents; diuretics; emollients; enzymes; erythropoietic drugs; expectorants; fertility agents; fungicides; gastrointestinal agents; growth regulators; hormone replacement agents; hyperglycemic agents; hypnotics; hypoglycemic agents; laxatives; migraine treatments; mucolytics; narcotics; neuroleptics; neuromuscular drugs; NSAIDS; peripheral vasodilators; prostaglandins; psychotropics; renin inhibitors; respiratory stimulants; steroids; stimulants; sympatholytics; thyroid preparations; tranquilizers; uterine relaxants; vaginal preparations; vasoconstrictors; vasodilators; vertigo agents; vitamins; and wound healing agents.

In some embodiments, Z is a therapeutic moiety obtained from a therapeutic agent selected from the group consisting of an antibiotic, an anti-inflammatory agent, an anti-viral agent, an anti-cancer agent, an anti-infective agent, and combinations thereof. In some embodiments, Z is a therapeutic moiety obtained from a corticosteroid. Non-limiting examples of corticosteroids include dexamethasone, prednisone, prednisolone, cortisone, hydrocortisone, betamethasone, and combinations thereof. In certain embodiments, Z is a dexamethasone residue. In some embodiments, Z is a therapeutic moiety obtained from an antibiotic or an anti-cancer agent. In some embodiments, Z is a mithramycin residue or a doxorubicin residue.

In some embodiments, a compound having a formula (I) or a pharmaceutically acceptable salt thereof is presented, wherein Z is a therapeutic moiety obtained from a corticosteroid. In some embodiments, a compound having a formula (I) or a pharmaceutically acceptable salt thereof is presented, wherein Z is a therapeutic moiety obtained from a corticosteroid selected from the group consisting of dexamethasone, prednisone, prednisolone, triamcinolone, cortisone, hydrocortisone, and betamethasone. In some embodiments, a compound having a formula (I) or a pharmaceutically acceptable salt thereof is presented, wherein Z is a dexamethasone residue.

In some embodiments, compounds having a formula (IX) to (XIII) or pharmaceutically acceptable salts thereof are presented, wherein Z is a therapeutic moiety obtained from a corticosteroid. In some embodiments, compounds having a formula (IX) to (XIII) or pharmaceutically acceptable salts thereof are presented, wherein Z is a therapeutic moiety obtained from a corticosteroid selected from the group consisting of dexamethasone, prednisone, prednisolone, triamcinolone, cortisone, hydrocortisone, and betamethasone. In some embodiments, a compound having a formula (IX) to (XIII) or a pharmaceutically acceptable salts thereof are presented, wherein Z is a dexamethasone residue.

In some embodiments, a pharmaceutical composition is presented. The pharmaceutical composition includes a compound as described herein above or a pharmaceutically acceptable salt, solvate, hydrate, polymorph, or co-crystal thereof, and a pharmaceutical carrier, diluent, or excipient. In some embodiments, the pharmaceutical composition includes a compound having a formula (I). In some embodiments, the pharmaceutical composition includes a compound having a formula (IX) to (XIII).

The pharmaceutical compositions of the present invention may be in any form that allows for the composition to be administered to a subject. For example, the composition may be in the form of a solid, liquid, or gas (aerosol). Pharmaceutical compositions may be formulated to allow the active ingredients contained therein to be bioavailable upon administration of the composition to a subject. To further optimize the pharmacokinetic profile of the compounds of the present invention, the compounds may be administered in conjunction with a suitable delivery vehicle (e.g., microcapsules, microspheres, biodegradable polymer films, lipid-based delivery systems such as liposomes and lipid foams, viscous instillates and absorbable mechanical barriers) useful for maintaining the necessary concentrations of the prodrugs or the therapeutic agent at the site of the disease.

A process for preparing a pharmaceutical composition is also presented. The process includes mixing a compound as described hereinabove or a pharmaceutically acceptable salt, solvate, hydrate, polymorph, or co-crystal thereof with a pharmaceutically acceptable carrier, diluent, or excipient. In some embodiments, the process includes mixing a compound having a formula (I) or a pharmaceutically acceptable salt, solvate, hydrate, polymorph, or co-crystal thereof with a pharmaceutically acceptable carrier, diluent, or excipient. In some embodiments, the process includes mixing a compound having a formula (IX) to (XIII) or a pharmaceutically acceptable salt, solvate, hydrate, polymorph, or co-crystal thereof with a pharmaceutically acceptable carrier, diluent, or excipient.

In some embodiments, methods of treating or reducing symptoms of a certain disease by administering a compound of the present invention are also presented. The compounds or derivatives thereof can be administered to any host, including a human, a non-human animal, and mammals, in an amount effective to treat a disorder.

In some embodiments, a method of treating an inflammation is presented. The method includes administering to a patient an effective amount of a pharmaceutical composition including a compound of the present invention or a pharmaceutically acceptable salt, solvate, hydrate, polymorph, or co-crystal thereof. In some embodiments, the inflammation is a lung inflammation.

In some embodiments, a method of treating a chronic respiratory disease is presented. The method includes administering to a patient an effective amount of a pharmaceutical composition including a compound of the present invention or a pharmaceutically acceptable salt, solvate, hydrate, polymorph, or co-crystal thereof.

Chronic respiratory diseases (CRDs) and noncommunicable diseases are the leading cause of death and disability globally. The major CRDs in adult disease include chronic obstructive pulmonary diseases (COPD), sarcoidosis, eosinophilic asthma, and other types of asthma. COPD and asthma are associated with chronic inflammation in the airways and parenchyma. Non-limiting examples of chronic respiratory disease include chronic obstructive pulmonary disease (COPD), sarcoidosis, eosinophilic asthma, or other asthma.

Corticosteroids are a class of pharmaceutically active agents used for the long-treatment of COPD and asthma. Two main classes of corticosteroids, glucocorticoids, and mineralocorticoids, are involved in a wide range of physiological processes, including stress response, immune response, and regulation of inflammation, carbohydrate metabolism, protein catabolism, blood electrolyte levels, and behavior. Glucocorticoids are one class of corticosteroids commonly used for the treatment of COPD, sarcoidosis, eosinophilic asthma, and other types of asthma. Glucocorticoids are small-molecule steroids that bind to glucocorticoid receptors (GRs) and are utilized in anti-inflammatory and immunosuppressive therapies. In sarcoidosis, asthma, and COPD, the greater expression of inflammatory genes is regulated by pro-inflammatory transcription factors, which bind to the acetylated core histones of coactivator molecules to activate them, thereby initiating the transcription of inflammatory genes. Glucocorticoids play a role in repressing pro-inflammatory genes and activating anti-inflammatory genes that have been triggered by pro-inflammatory stimuli. However, due to the ubiquitous expression of glucocorticoid receptors in many cell types, glucocorticoid treatments are compromised by toxicities to most organ systems. Some embodiments of the present invention address the noted shortcomings in the art by providing prodrug compounds capable of specifically transporting and releasing the drug within the lung and thereby reducing toxicity.

In some embodiments, a method of treating edema is presented. The method includes administering to a patient an effective amount of a pharmaceutical composition including a compound of the present invention or a pharmaceutically acceptable salt, solvate, hydrate, polymorph, or co-crystal thereof. In some embodiments, the edema includes peripheral, pulmonary, or cerebral edema.

In some embodiments, a method of treating a brain disease is presented. The method includes administering to a patient an effective amount of a pharmaceutical composition including a compound of the present invention or a pharmaceutically acceptable salt, solvate, hydrate, polymorph, or co-crystal thereof. In some embodiments, brain disease is glioblastoma, glioma, medulloblastoma, or metastatic brain disease.

The blood-brain barrier (BBB) is a system-wide membrane barrier that prevents the brain uptake of circulating drugs, protein therapeutics, RNAi drugs, and gene medicines. Drugs or genes can be delivered to the human brain for the treatment of serious brain disease either (a) by injecting the drug or gene directly into the brain, thus bypassing the BBB, or (b) by injecting the drug or gene into the bloodstream so that the drug or gene enters the brain via the transvascular route across the BBB. Intra-cerebral administration of the drug is highly invasive and not very effective. The transvascular route is non-invasive and can potentially allow for wider distribution of the drug to the target cells in the brain. However, this latter approach requires the ability to undergo transport across the BBB, which has been a difficult barrier to traverse safely. Some embodiments of the present invention address the noted shortcomings in the art by providing prodrug compounds capable of specifically transporting the drug across BBB and releasing the drug within the brain and thereby reducing toxicity.

The pharmaceutical composition may be administered by any suitable method known to a person skilled in the art. Typical routes of administration include, without limitation, oral, topical, parenteral, sublingual, rectal, vaginal, ocular, and intranasal. The term parenteral as used herein includes intravenous, intraperitoneal, intramuscular, intradermal, and epidermal including subcutaneous and intradermal, oral, or application to mucosal surfaces, e.g, by intranasal administration using inhalation of aerosol suspensions, and by implanting to muscle or other tissue in the subject.

### SPECIFIC EMBODIMENTS

The following enumerated embodiments are representative of some aspects of the invention.

Embodiment 1. A compound having a structure of formula (I), or a pharmaceutically acceptable salt thereof, wherein formula (I) is:

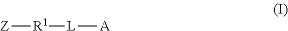

wherein Z is a therapeutic moiety;
L is a linker moiety bonded to Z via R¹, wherein R¹ is O, N, or NH, and L is selected from:

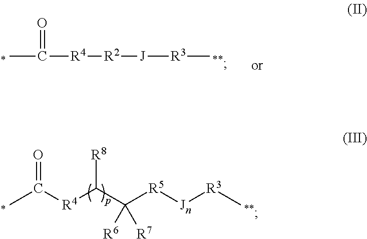

wherein * denotes the point of attachment of the linker moiety to R¹ and ** denotes the point of attachment of the linker moiety to A;
J is an aryl group or a heteroaryl group optionally substituted with one or more functional groups;
R² and R³ are independently at each occurrence a bond, a C₁-C₆ alkylene group, or a C₁-C₆ alkenylene group;
R⁴ is a bond, O or NR′;
R⁵ is a bond, a C₁-C₆ alkylene group, O or NR′, wherein R′ is independently hydrogen or a C₁-C₃ alkyl group;
R⁶, R⁷, and R⁸ are independently at each occurrence hydrogen or a C₁-C₃ alkyl group, with the proviso that at least one R⁶, R⁷, or R⁸ is a C₁-C₃ alkyl group or R⁶ and R⁷ together with a carbon to which each is attached form a C₃-C₆ cycloalkyl group;
“n” is an integer from 0 to 1, “p” is an integer from 0 to 1; and
A is an amino acid moiety or a peptide moiety.

Embodiment 2 The compound of embodiment 1, wherein A comprises a residue of one or more α-amino acids selected from the group consisting of glycine, valine, isoleucine, proline, phenyl alanine, tryptophan, and combinations thereof.

Embodiment 3 The compound of embodiment 1 or embodiment 2, wherein the linker moiety is selected from:

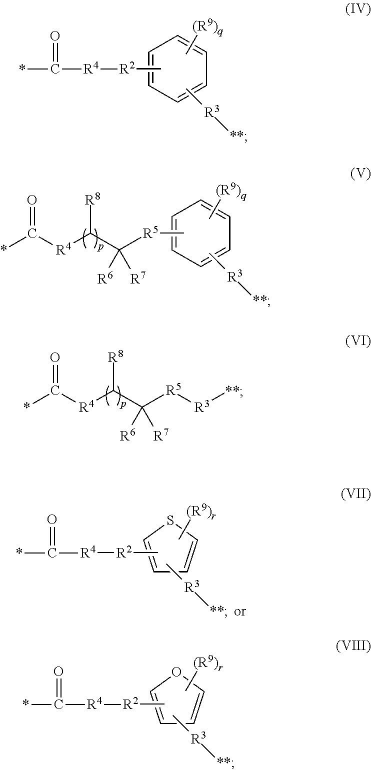

wherein “q” is an integer from 0 to 4, “Y” is an integer from 0 to 2; and R′ is independently at each occurrence a functional group selected from alkoxy, hydroxy, halogen, amine, nitro, cyano, or dialkylamine.

Embodiment 4 The compound of any one of embodiments 1-3, having a structure of formula (IX) to (XIII), or a pharmaceutically acceptable salt thereof:

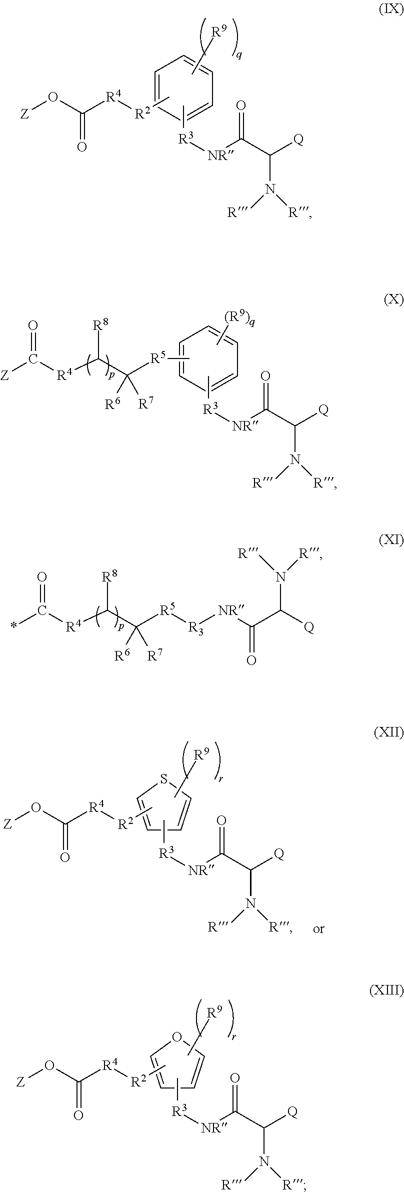

wherein “q” is an integer from 0 to 4, “r” is an integer from 0 to 2;
R⁹ is independently at each occurrence a functional group selected from alkoxy, hydroxy, halogen, amine, nitro, cyano, or dialkylamine;
R″ is hydrogen or a C₁-C₃ alkyl group; and
R′″ is independently at each occurrence hydrogen, a C₁-C₃ alkyl group, or a C(═O)CH(Q)NR′″ moiety, Q is an amino acid side chain.

Embodiment 5 The compound of any one of embodiments 1-4, wherein R², R³, and R⁵ are independently a bond or a C₁-C₃ alkylene group, R⁴ is a bond, O, or NH, R⁶, and R⁷ are independently at each occurrence a methyl group, or R⁶ and R⁷ together with a carbon to which each is attached form a cyclopropyl group, and “p” is 0 or R⁸ is a C₁-C₃ alkyl group.

Embodiment 6 The compound of any one of embodiments 1-5, wherein R², R³, and R⁵ are independently a bond or a C₁-C₃ alkylene group, R⁴ is a bond, R⁶, and R⁷ are independently at each occurrence a methyl group, or R⁶ and R⁷ together with a carbon to which each is attached form a cyclopropyl group, and “p” is 0 or R⁸ is a C₁-C₃ alkyl group.

Embodiment 7 The compound of any one of embodiments 1-6, wherein R², R³, R⁴, and R⁵ are independently a bond, R⁶ and R⁷ are independently at each occurrence a methyl group, or R⁶ and R⁷ together with a carbon to which each is attached form a cyclopropyl group, and “p” is 0 or R⁸ is a methyl group.

Embodiment 8 The compound of any one of embodiments 3-7, wherein “q” is 1-2, “r” is 1-2, and R⁹ is independently at each occurrence alkoxy, halogen, amine, or dialkylamine.

Embodiment 9 The compound of any one of embodiments 4-8, wherein Q is independently at each occurrence an amino acid side chain of an amino acid selected from the group consisting of valine, proline, phenyl alanine, and tryptophan.

Embodiment 10 The compound of any one of embodiments 1-3, when Z is a therapeutic moiety obtained from a therapeutic agent selected from the group consisting of an antibiotic, an anti-inflammatory agent, an anti-viral agent, an anti-cancer agent, an anti-infective agent, and combinations thereof.

Embodiment 11 The compound of any one of embodiments 1-3 and 10, wherein Z is a therapeutic moiety obtained from a corticosteroid.

Embodiment 12 The compound of any one of embodiments 1-3 and 10, wherein the corticosteroid is selected from the group consisting of dexamethasone, prednisone, prednisolone, triamcinolone, cortisone, hydrocortisone, and betamethasone.

Embodiment 13 The compound of any one of embodiments 4-9, wherein Z is a therapeutic moiety obtained from a therapeutic agent selected from the group consisting of an antibiotic, an anti-inflammatory agent, an anti-viral agent, an anti-cancer agent, an anti-infective agent, and combinations thereof.

Embodiment 14 The compound of any one of embodiments 4-9 and 13, wherein Z is a therapeutic moiety obtained from a corticosteroid selected from the group consisting of dexamethasone, prednisone, prednisolone, triamcinolone, cortisone, hydrocortisone, and betamethasone.

Embodiment 15 The compound of any one of embodiments 4-9 and 13-14, wherein Z is a dexamethasone residue.

Embodiment 16 A compound selected from the group consisting of:

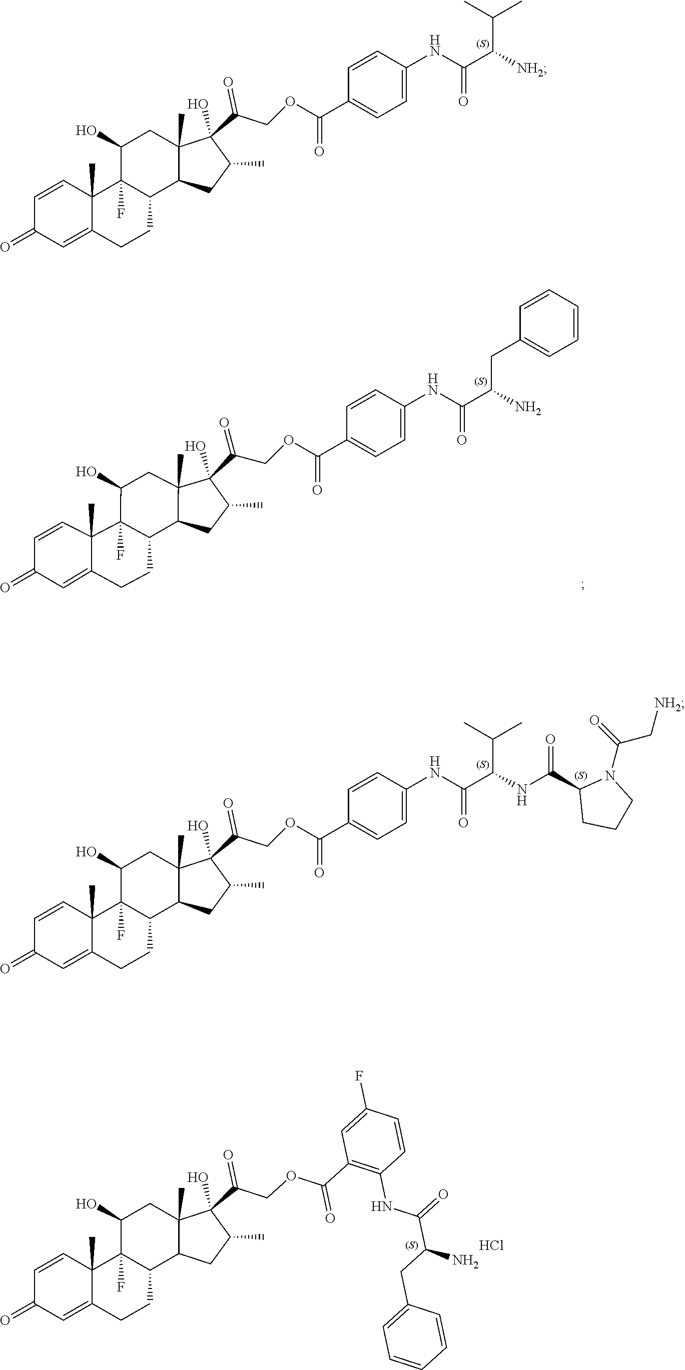

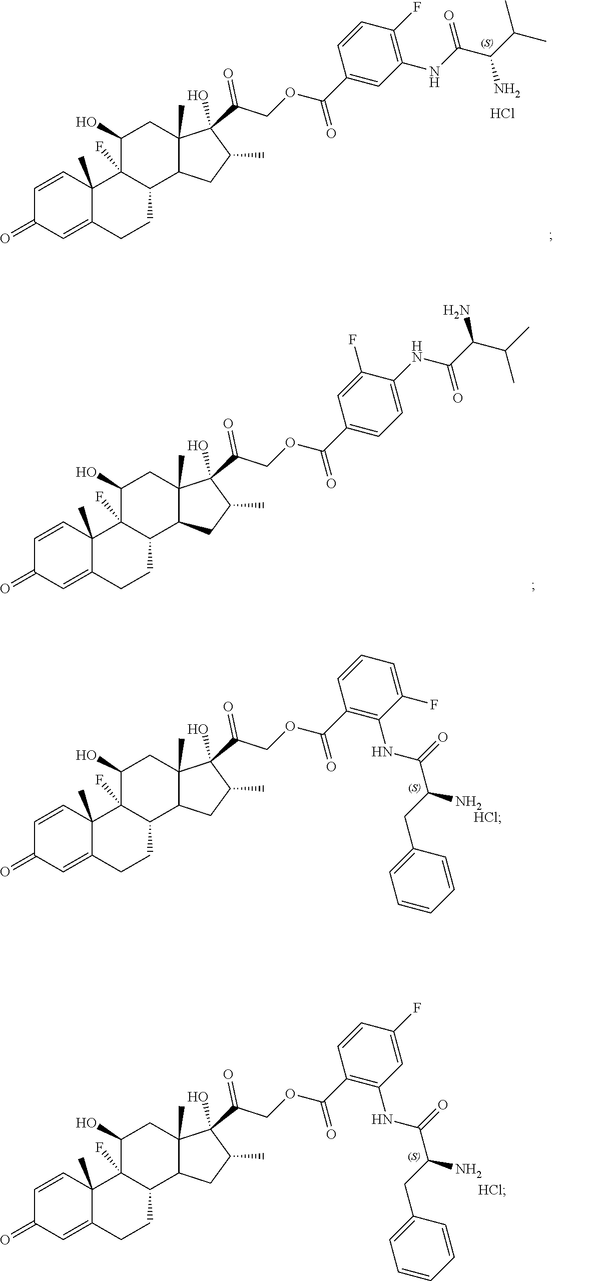

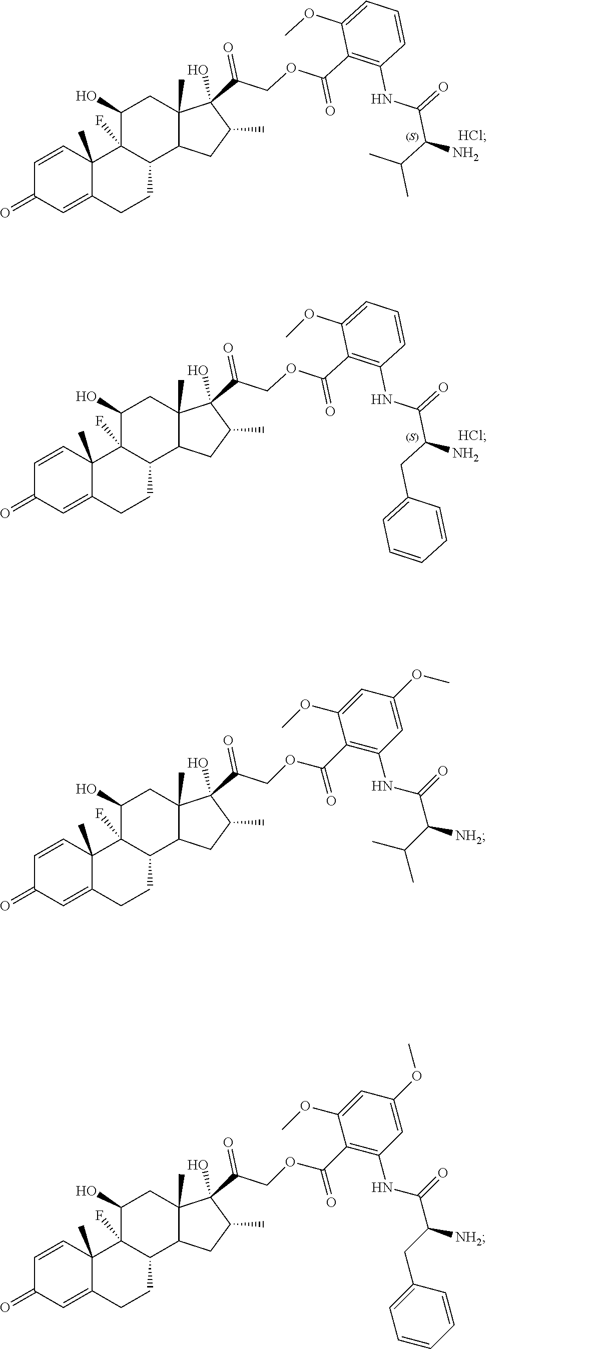

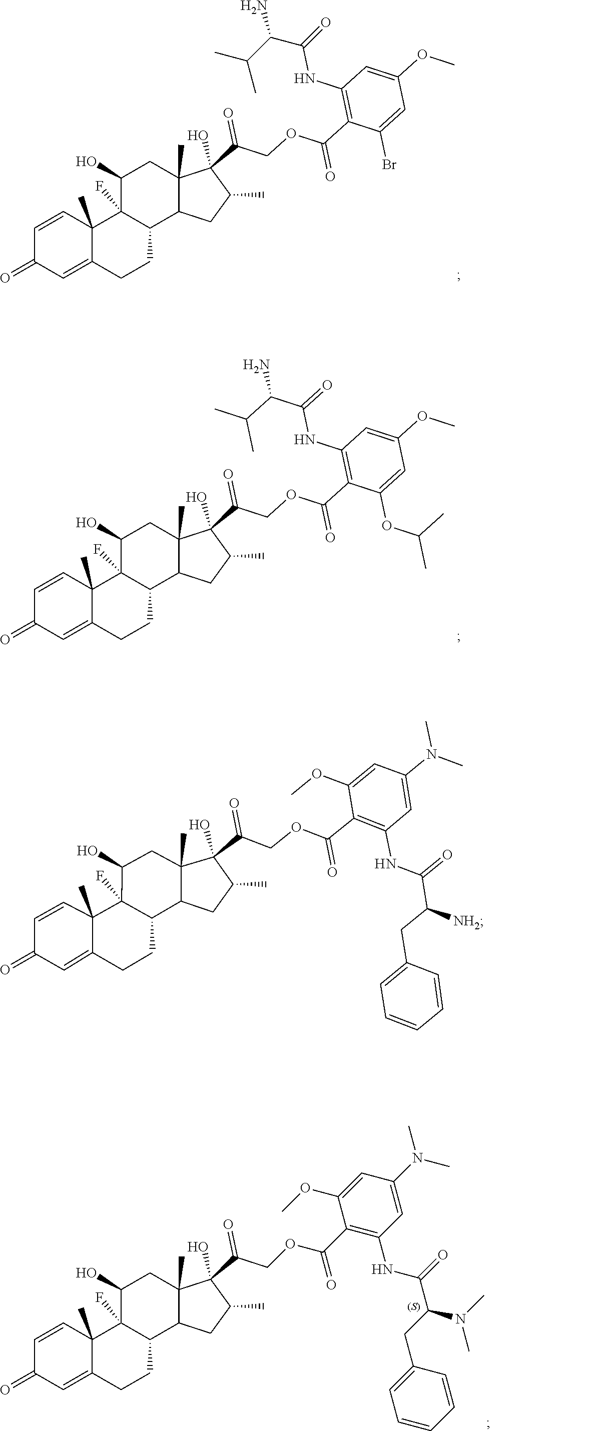

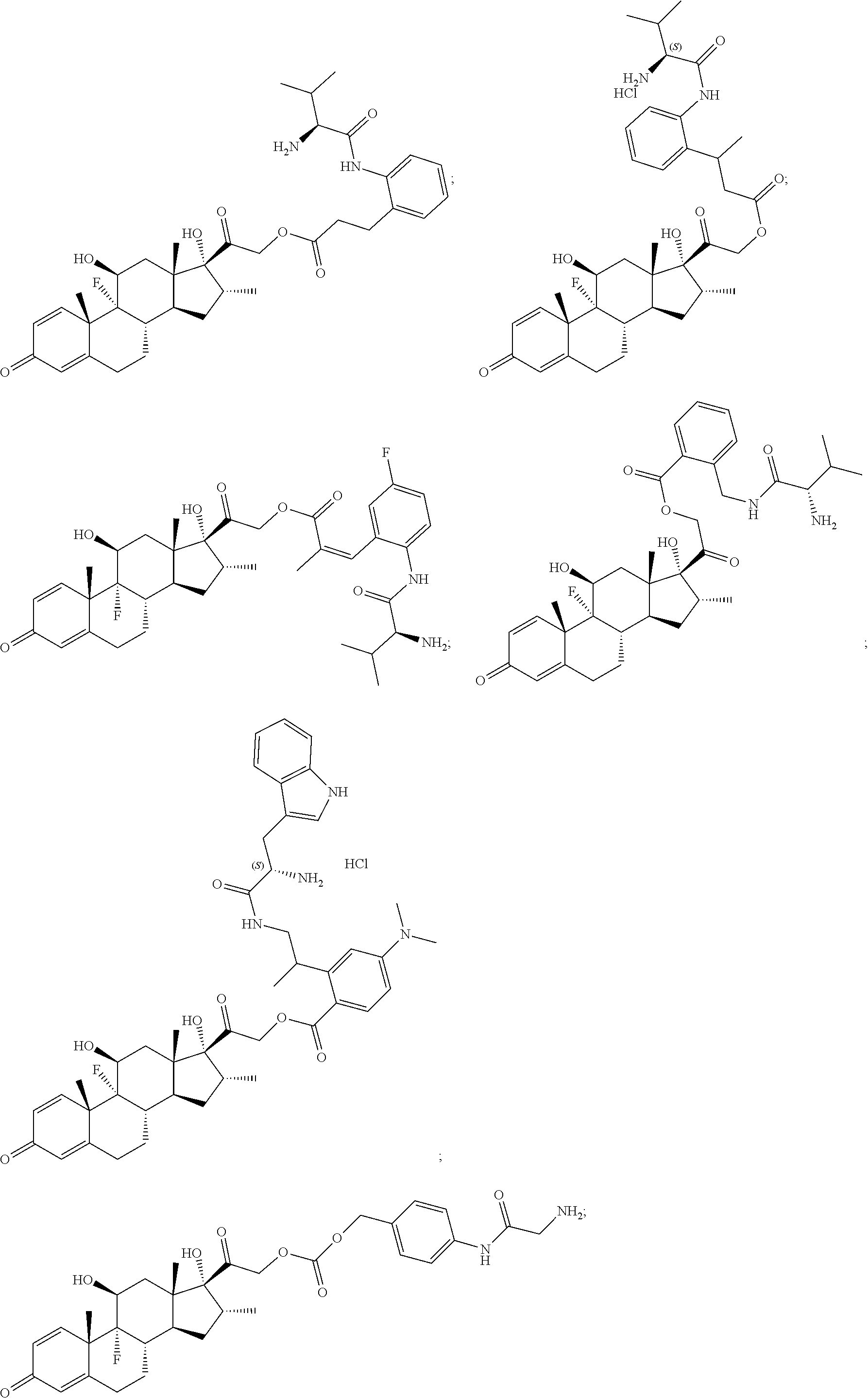

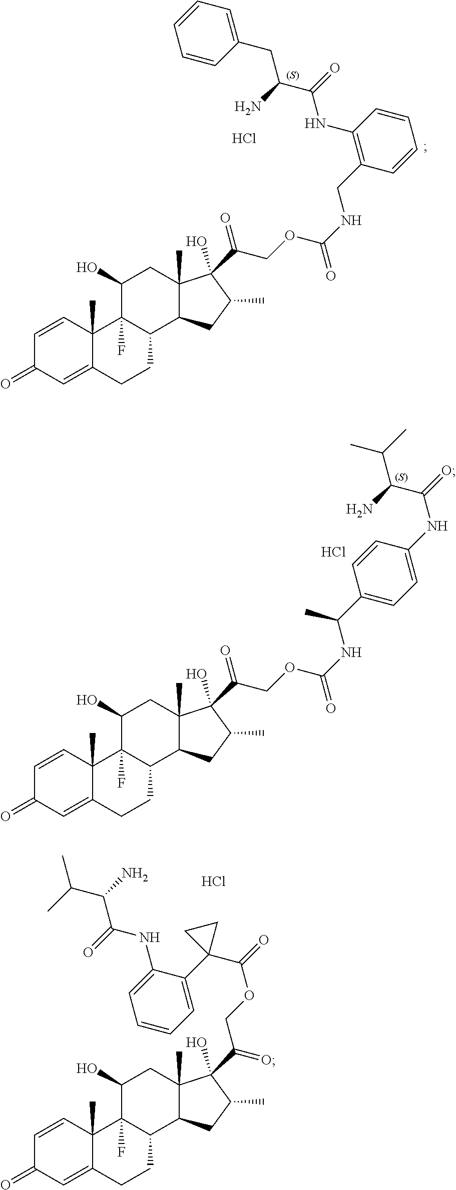

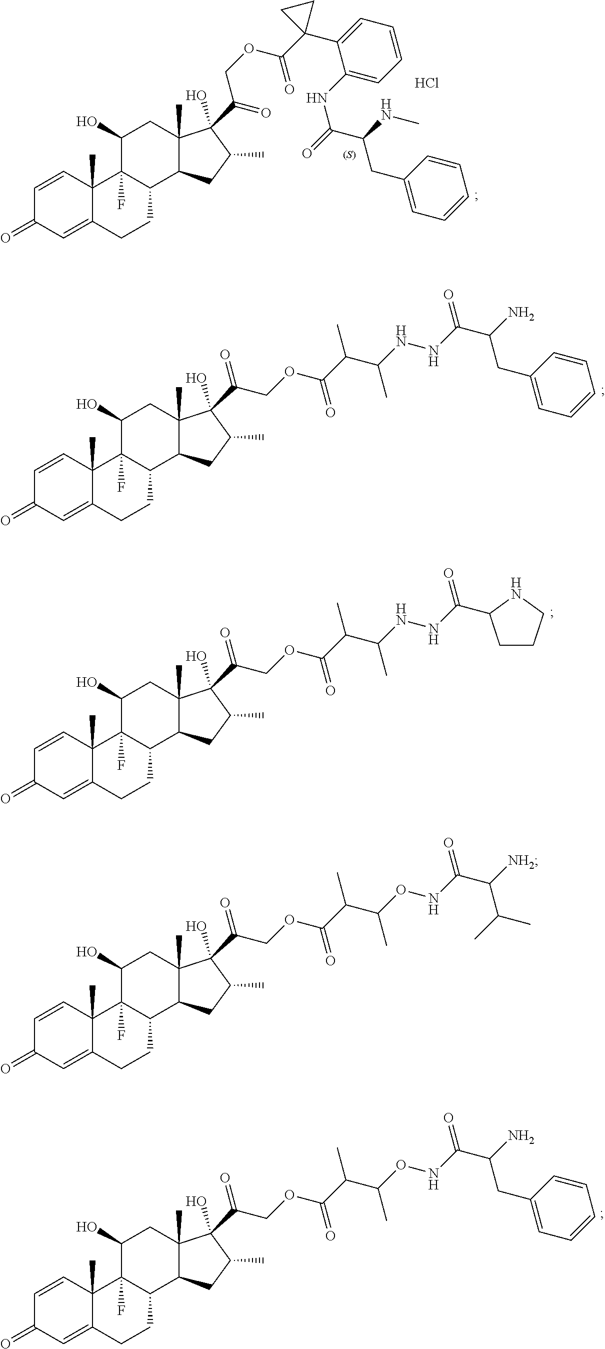

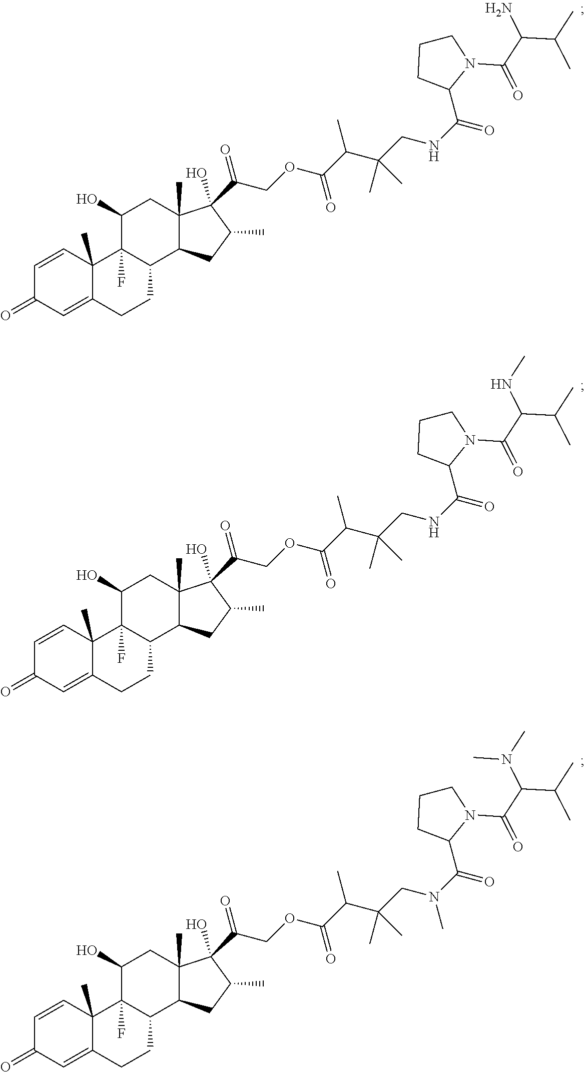

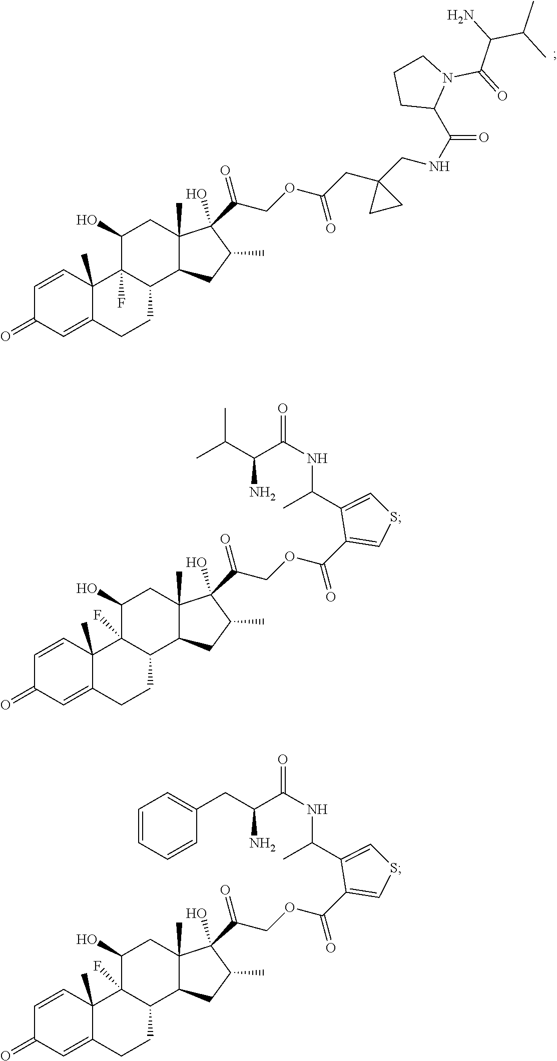

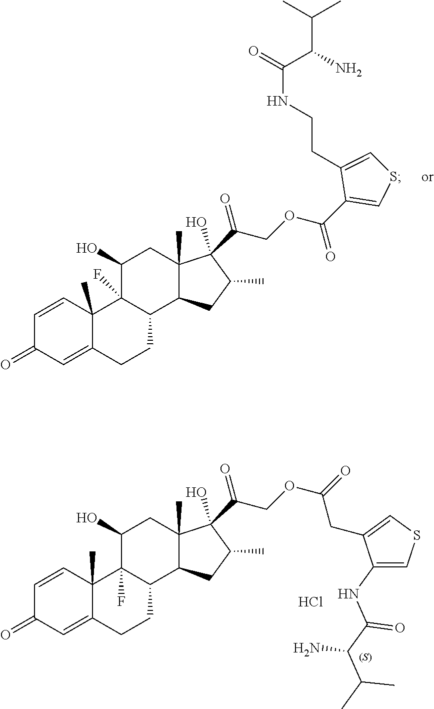

Embodiment 17 A pharmaceutical composition comprising:

the compound of any one of embodiments 1-16 or a pharmaceutically acceptable salt, solvate, hydrate, polymorph, or co-crystal thereof, and a pharmaceutically carrier, diluent, or excipient.

Embodiment 18 A method of treating a chronic respiratory disease, edema, or brain disease comprising administering to a patient an effective amount of a pharmaceutical composition comprising a compound of any one of embodiments 1-16 or a pharmaceutically acceptable salt, solvate, hydrate, polymorph, or co-crystal thereof.

Embodiment 19 The method of embodiment 18, wherein the chronic respiratory disease is chronic obstructive pulmonary disease (COPD), sarcoidosis, or asthma.

Embodiment 20 The method of embodiment 18, wherein the edema is cerebral edema, pulmonary edema, or peripheral edema.

Embodiment 21 The method of embodiment 18, wherein the brain disease is glioblastoma, medulloblastoma, glioma, or brain metastatic disease.

### EXAMPLES

### General Methods for the Synthesis of Prodrugs

Dexamethasone (>95% purity) was purchased from a commercial source and used for the conjugation with linkers. All commercial reagents and solvents were used as received. Reaction progress, intermediates and final products were monitored and assessed using LC/MS, HPLC-UV and by NMR (1H and 13C). Purification of the prodrug was performed on a silica gel column using flash column chromatography or by prep-LC when required. The ¹H NMR spectra was recorded on a Bruker BioSpin GmbH spectrometer at 300 MHz. Coupling constants (J) are reported in hertz (Hz). Chemical shifts (δ) are reported in parts per million referenced with respect to the residual solvent (DMSO-d6) 2.50 ppm or from internal standard tetramethylsilane 0.00 ppm. The following abbreviations were used in the reporting spectra: s, singlet; d, doublet; t, triplet; q, quartet; m, multiplet; dd, doublet of doublets; td, triplet of doublets; ddd, doublet of doublet of doublets. All prodrugs synthesized in this project were >95% pure.

Non-limiting examples of compounds synthesized according to the experimental section described herein are listed in Table 1.

| Compound No | Compound Structure |
|---|---|
| Comparative Compound 1 | 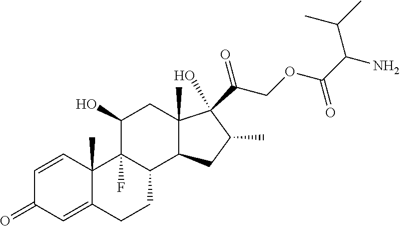 |
| Comparative Compound 2 | 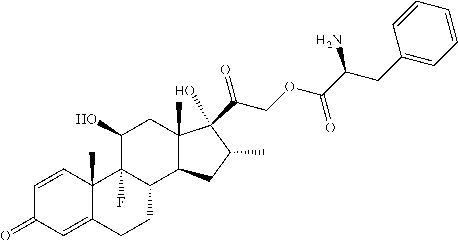 |
| Comparative Compound 3 | 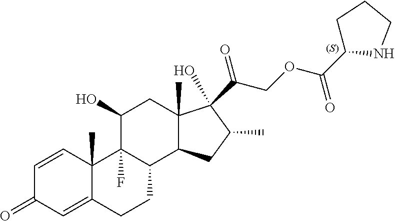 |
| Comparative Compound 4 | 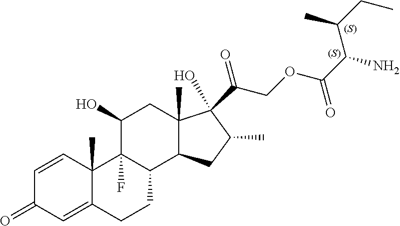 |
| Compound 1 | 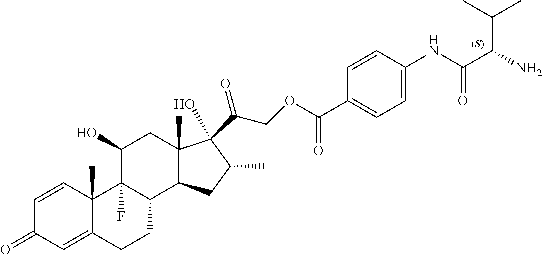 |
| Compound 2 | 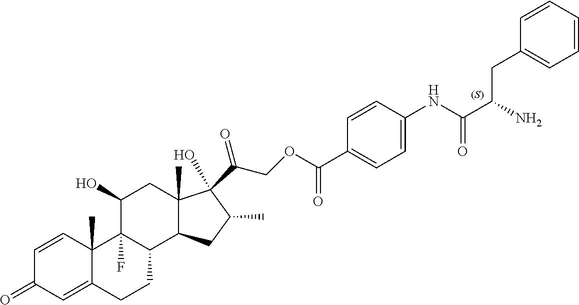 |
| Compound 3 | 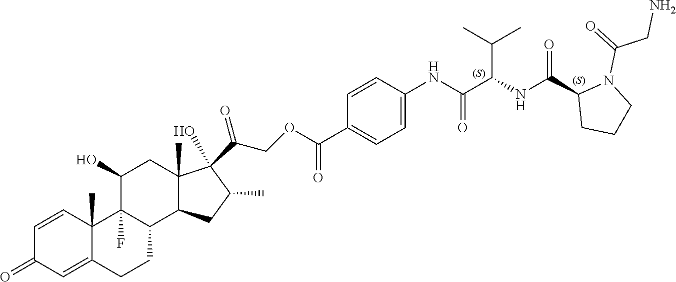 |
| Compound 4 | 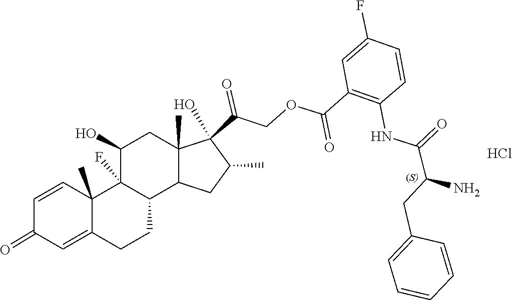 |
| Compound 5 | 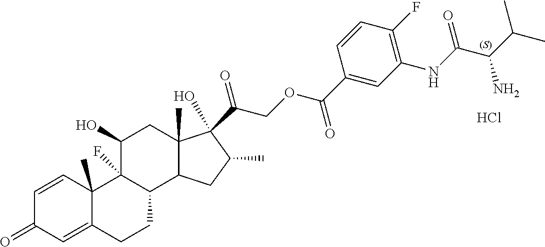 |
| Compound 6 | 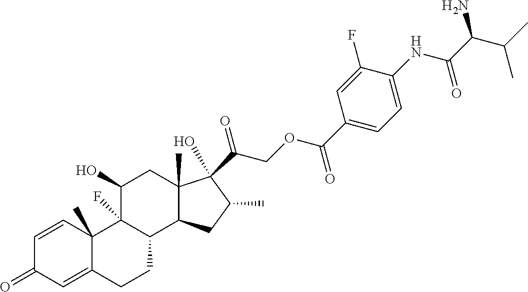 [SMILES: CC(C)[C@H](N)C(=O)Nc1ccc(C(=O)OCC(=O)[C@@]2(O)[C@H](C)C[C@H]3[C@@H]4CCC5=CC(=O)C=C[C@]5(C)[C@@]4(F)[C@@H](O)C[C@@]32C)cc1F] |
| Compound 7 | 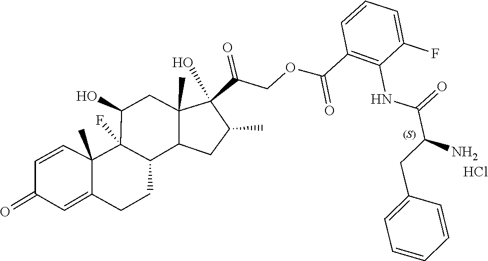 |
| Compound 8 | 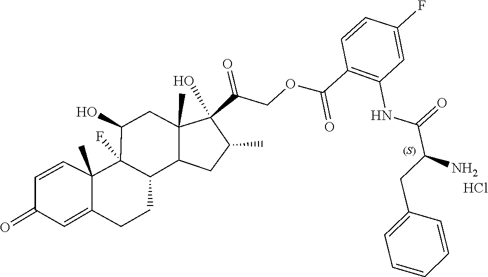 |
| Compound 9 | 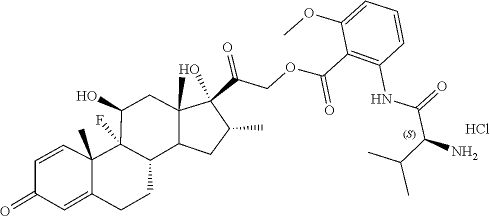 |
| Compound 10 | 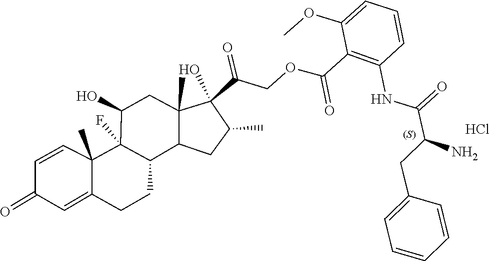 |
| Compound 11 | 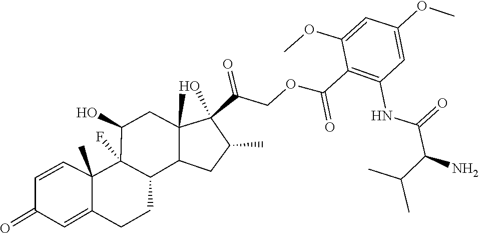 [SMILES: COc1cc(NC(=O)[C@@H](N)C(C)C)c(C(=O)OCC(=O)[C@@]2(O)[C@H](C)CC3[C@@H]4CCC5=CC(=O)C=C[C@]5(C)[C@@]4(F)[C@@H](O)C[C@@]32C)c(OC)c1] |
| Compound 12 | 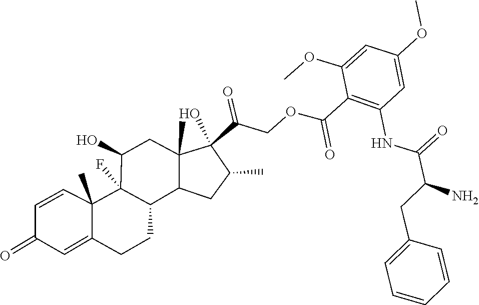 [SMILES: COc1cc(NC(=O)[C@@H](N)Cc2ccccc2)c(C(=O)OCC(=O)[C@@]2(O)[C@H](C)CC3[C@@H]4CCC5=CC(=O)C=C[C@]5(C)[C@@]4(F)[C@@H](O)C[C@@]32C)c(OC)c1] |
| Compound 13 | 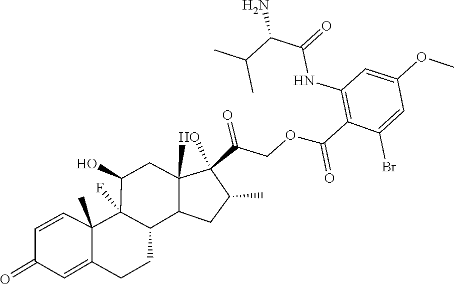 [SMILES: COc1cc(Br)c(C(=O)OCC(=O)[C@@]2(O)[C@H](C)CC3[C@@H]4CCC5=CC(=O)C=C[C@]5(C)[C@@]4(F)[C@@H](O)C[C@@]32C)c(NC(=O)[C@@H](N)C(C)C)c1] |
| Compound 14 | 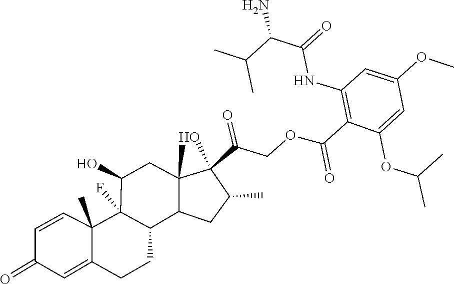 [SMILES: COc1cc(NC(=O)[C@@H](N)C(C)C)c(C(=O)OCC(=O)[C@@]2(O)[C@H](C)CC3[C@@H]4CCC5=CC(=O)C=C[C@]5(C)[C@@]4(F)[C@@H](O)C[C@@]32C)c(OC(C)C)c1] |
| Compound 15 | 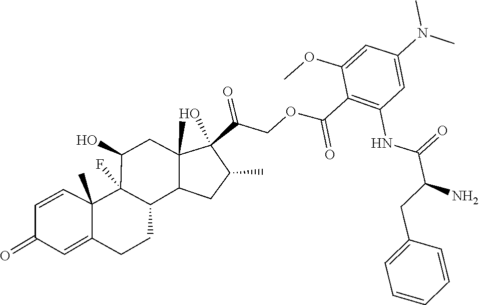 [SMILES: COc1cc(N(C)C)cc(NC(=O)[C@@H](N)Cc2ccccc2)c1C(=O)OCC(=O)[C@@]1(O)[C@H](C)CC2[C@@H]3CCC4=CC(=O)C=C[C@]4(C)[C@@]3(F)[C@@H](O)C[C@@]21C] |
| Compound 16 | 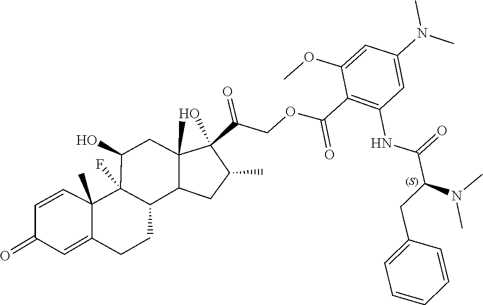 |
| Compound 17 | 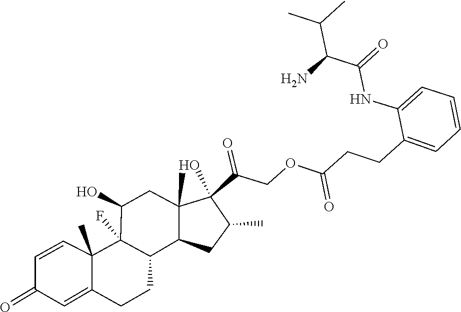 [SMILES: CC(C)[C@H](N)C(=O)Nc1ccccc1CCC(=O)OCC(=O)[C@@]1(O)[C@H](C)C[C@H]2[C@@H]3CCC4=CC(=O)C=C[C@]4(C)[C@@]3(F)[C@@H](O)C[C@@]21C] |
| Compound 18 | 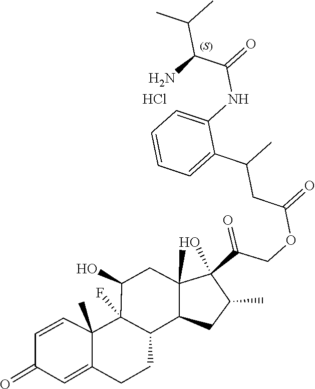 |
| Compound 19 | 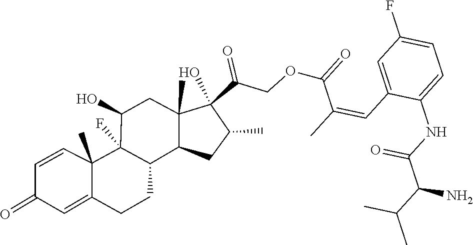 [SMILES: C/C(=C/c1cc(F)ccc1NC(=O)[C@@H](N)C(C)C)C(=O)OCC(=O)[C@@]1(O)[C@H](C)C[C@H]2[C@@H]3CCC4=CC(=O)C=C[C@]4(C)[C@@]3(F)[C@@H](O)C[C@@]21C] |
| Compound 20 | 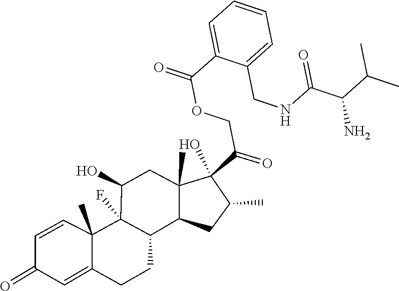 [SMILES: CC(C)[C@H](N)C(=O)NCc1ccccc1C(=O)OCC(=O)[C@@]1(O)[C@H](C)C[C@H]2[C@@H]3CCC4=CC(=O)C=C[C@]4(C)[C@@]3(F)[C@@H](O)C[C@@]21C] |
| Compound 21 | 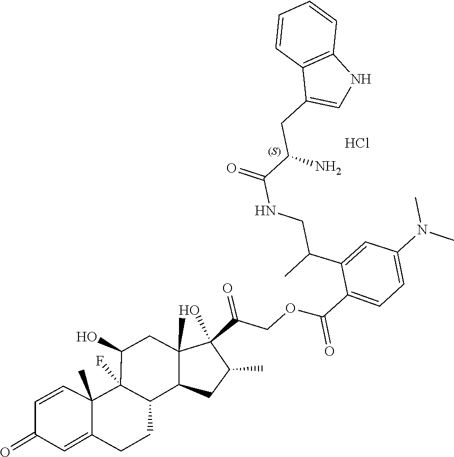 |
| Compound 22 | 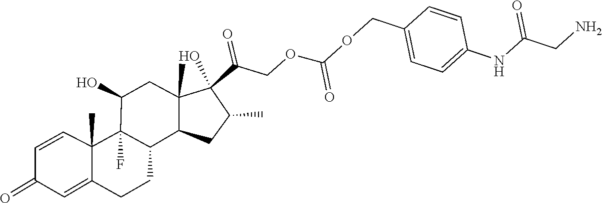 |
| Compound 23 | 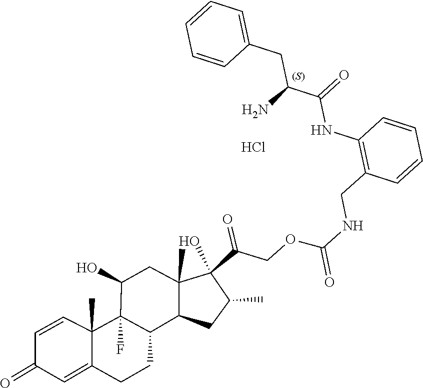 |
| Compound 24 | 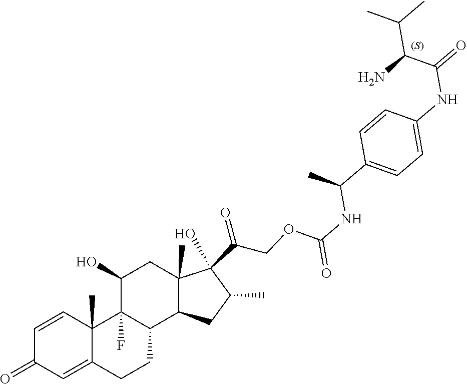 |
| Compound 25 | 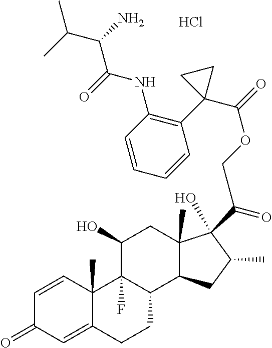 |
| Compound 26 | 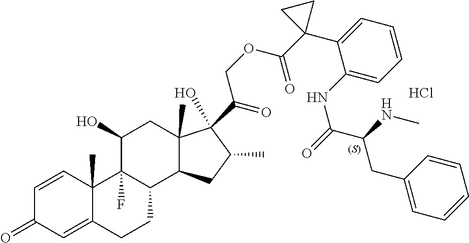 |
| Compound 27 | 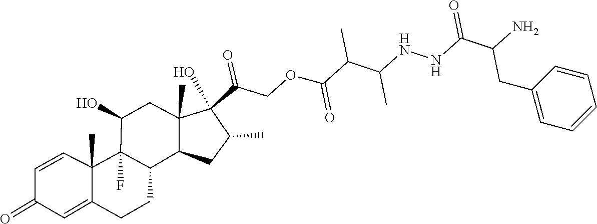 |
| Compound 28 | 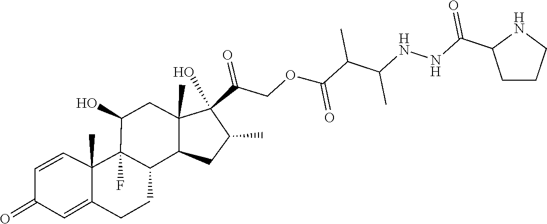 |
| Compound 29 | 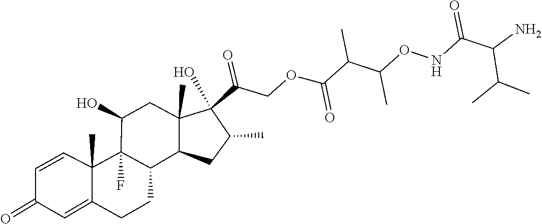 |
| Compound 30 | 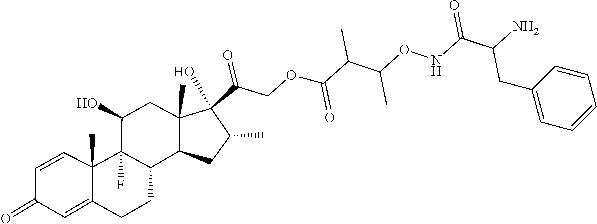 |
| Compound 31 | 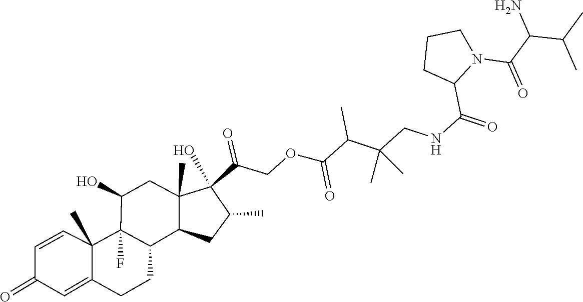 |
| Compound 32 | 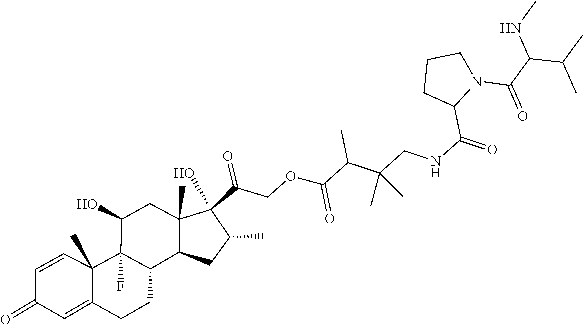 |
| Compound 33 | 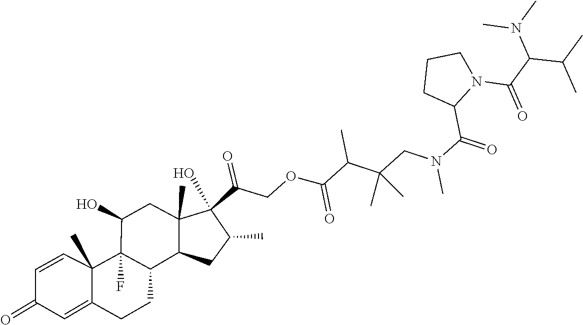 |
| Compound 34 | 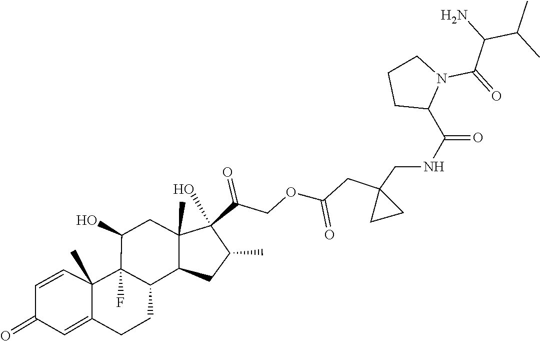 |
| Compound 35 | 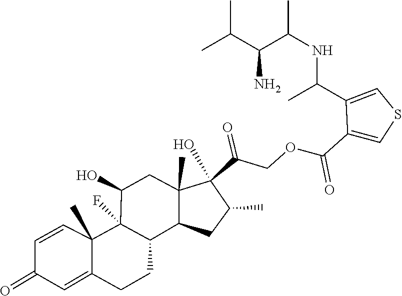 [SMILES: CC(NC(C)[C@@H](N)C(C)C)c1cscc1C(=O)OCC(=O)[C@@]1(O)[C@H](C)C[C@H]2[C@@H]3CCC4=CC(=O)C=C[C@]4(C)[C@@]3(F)[C@@H](O)C[C@@]21C] |
| Compound 36 | 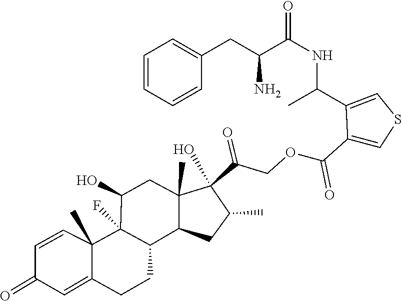 [SMILES: CC(NC(=O)[C@@H](N)Cc1ccccc1)c1cscc1C(=O)OCC(=O)[C@@]1(O)[C@H](C)C[C@H]2[C@@H]3CCC4=CC(=O)C=C[C@]4(C)[C@@]3(F)[C@@H](O)C[C@@]21C] |
| Compound 37 | 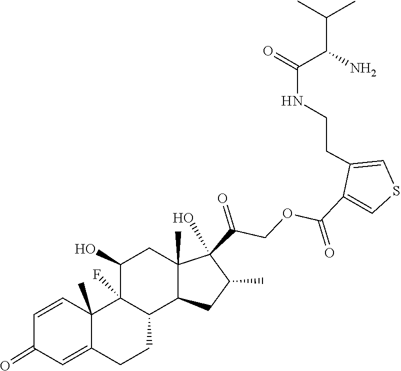 [SMILES: CC(C)[C@H](N)C(=O)NCCc1cscc1C(=O)OCC(=O)[C@@]1(O)[C@H](C)C[C@H]2[C@@H]3CCC4=CC(=O)C=C[C@]4(C)[C@@]3(F)[C@@H](O)C[C@@]21C] |
| Compound 38 | 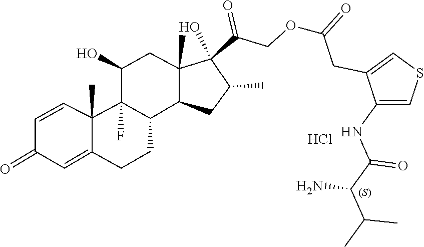 |

### Example 1: Synthesis of Comparative Compounds 1-4

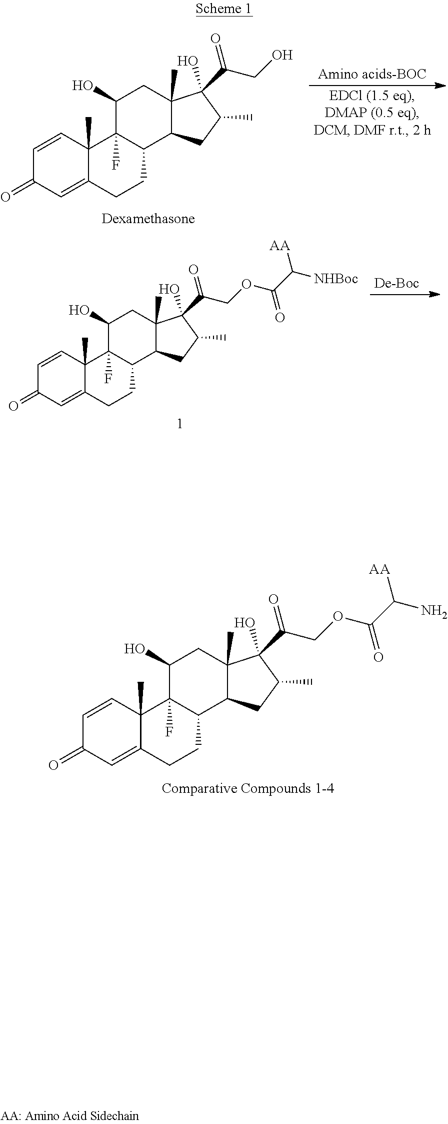

Comparative compounds 1-4 were synthesized according to Scheme 1 using valine, proline, phenylalanine and isoleucine amino acids. Into a round-bottom flask were added BOC-amino acids (1 equiv), DMF, DCM, dexamethasone (1 equiv) and DMAP (0.5 equiv) at room temperature. This was followed by addition of EDCI (1.5 equiv) at 0° C. The resulting mixture was stirred overnight at room temperature. The resulting mixture was concentrated under reduced pressure. The residue was purified by Prep-TLC (PE/EA 1:2) to afford the intermediate compound 1. Into a round-bottom flask were added intermediate compound 1, DCM/TFA (3/1) at room temperature. The resulting mixture was stirred for 2 h at room temperature. The resulting mixture was concentrated under reduced pressure. The crude product was purified by Prep-HPLC to afford the final product Comparative Compounds 1-4. Structures of the comparative compounds 1˜4 are listed in Table 1.

### Example 2: Synthesis of Compounds 1-2

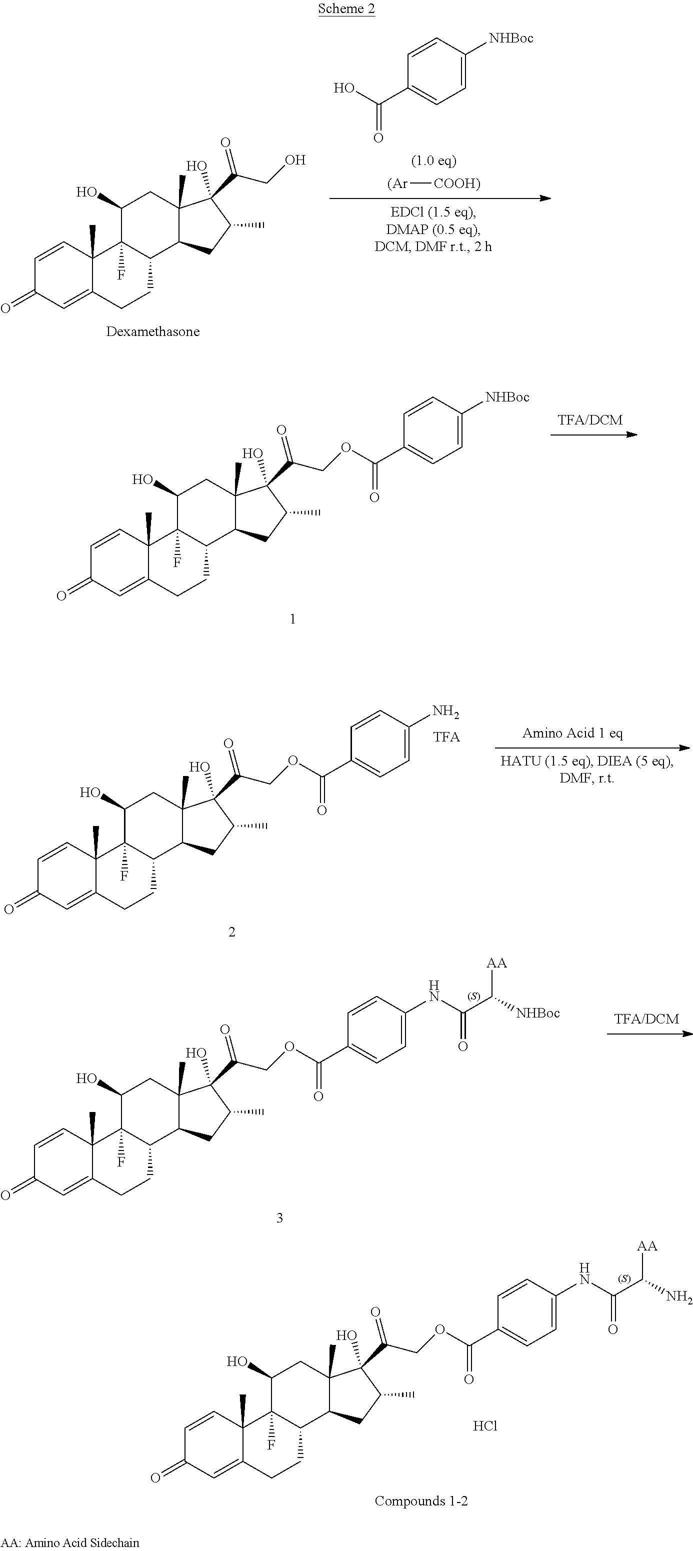

Compounds 1-2 were synthesized using scheme 2. Into a round-bottom flask were added Ar—COOH (1 equiv), DMF, DCM, dexamethasone (1 equiv) and DMAP (0.5 equiv) at room temperature. Then were added EDCI (1.5 equiv) at 0° C. The resulting mixture was stirred overnight at room temperature. The resulting mixture was concentrated under reduced pressure. The residue was purified by Prep-TLC (PE/EA 1:2) to afford the intermediate compound 1. Into a round-bottom flask were added intermediate compound 1, DCM/TFA (3/1) at room temperature. The resulting mixture was stirred for 2 h at room temperature. The resulting mixture was concentrated under reduced pressure. The crude product 2 was used in the next step directly without further purification. Into a round-bottom flask were added crude product 2 (1 equiv), DMF, amino acid (1.5 equiv), DIEA (5 equiv) and HATU (1.5 equiv) at room temperature. The resulting mixture was stirred for 1 h at room temperature. The resulting mixture was extracted with EtOAc. The combined organic layers were washed with brine, dried over anhydrous Na₂SO₄. After filtration, the filtrate was concentrated under reduced pressure. The residue was purified by Prep-TLC (PE/EA 1:1) to afford the intermediate product 3. Into a round-bottom flask were added intermediate compound 3, DCM/TFA (3/1) at room temperature. The resulting mixture was stirred for 2 h at room temperature. The resulting mixture was concentrated under reduced pressure. The crude product was purified by Prep-HPLC with the following conditions: Column, X-select CSH C18 OBD Column 30×150 mm 5 □m, mobile phase, Water (0.05% HCl) and ACN (13% ACN up to 43% in 8 min); Detector, UV254 nm product was obtained the final product Compounds 1-2. Structures of the compounds 1-2 are listed in Table 1. Compound 1: MS (ESI) (m/z): [M+1]⁺ calculated: 659.3; found: 659.4. Compound 2: MS (ESI) (m/z) [M+H]⁺ calculated: 610.2; found: 610.2.

### Example 3: Synthesis of Compound 3

Into a round-bottom flask were added Compound 1 (1 equiv) from Example 1, DMF, Proline (1.5 equiv), DIEA (5 equiv) and HATU (1.5 equiv) at room temperature. The resulting mixture was stirred for 1 h at room temperature. The resulting mixture was extracted with EtOAc. The combined organic layers were washed with brine, dried over anhydrous Na₂SO₄. After filtration, the filtrate was concentrated under reduced pressure. The residue was purified by Prep-TLC (PE/EA 1:1) to afford the intermediate compound 1. Into a round-bottom flask were added intermediate compound 1, DCM/TFA (3/1) at room temperature. The resulting mixture was stirred for 2 h at room temperature. The resulting mixture was concentrated under reduced pressure. The crude product was purified by Prep-HPLC to afford the final product Compound 3. Compound 3: MS (ESI) (m/z) [M+H]⁺ calculated: 765.4; found: 765.3.

### Example 4: Synthesis of Compounds 4-10

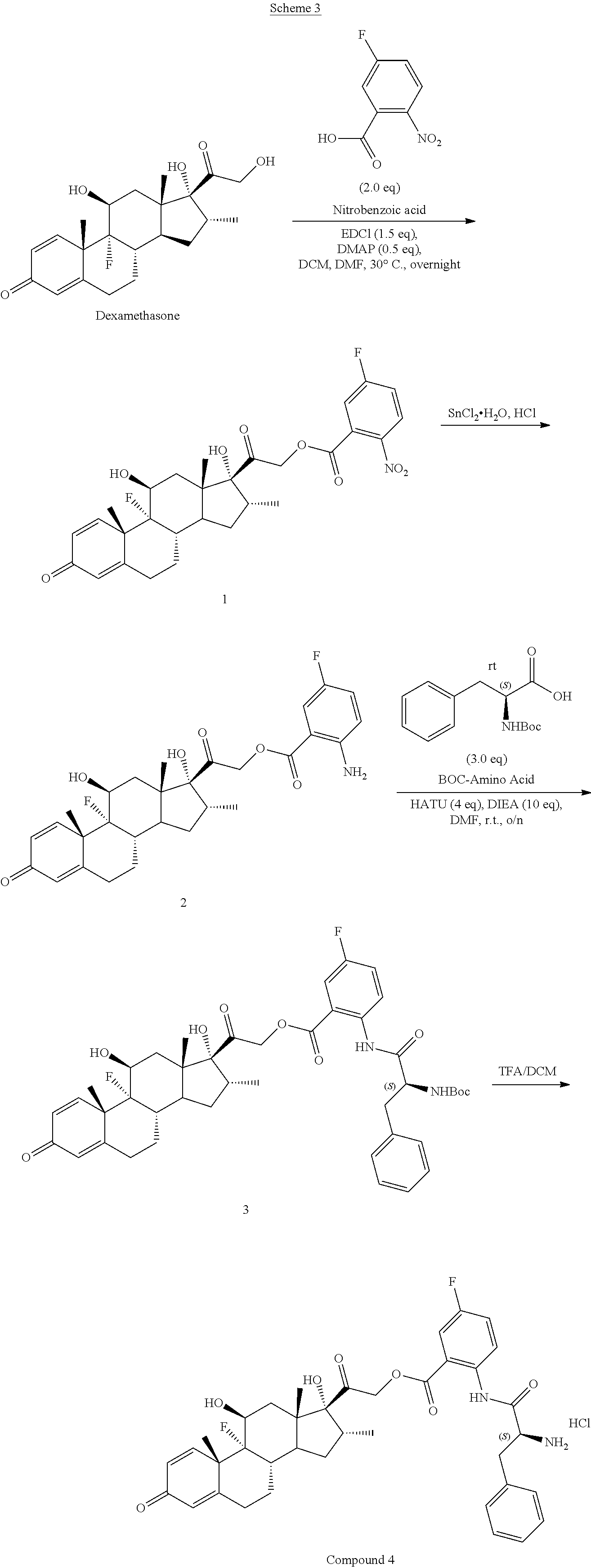

Into a round-bottom flask were added fluorinated or methoxylated nitrobenzoic acid (1 equiv), DMF, DCM, dexamethasone (1 equiv) and DMAP (0.5 equiv) at room temperature. Then were added EDCI (1.5 equiv) at 0° C. The resulting mixture was stirred overnight at room temperature. The resulting mixture was concentrated under reduced pressure. The residue was purified by Prep-TLC (PE/EA 1:2) to afford the intermediate compound 1.

Into a round-bottom flask were added intermediate compound 1 (1 equiv), SnCl₂·2H₂O (5 equiv) and HCl (12 N) at room temperature. The resulting mixture was stirred overnight at room temperature. The mixture was basified to pH 8 with saturated NaHCO₃ (aq.). The resulting mixture was extracted with EtOAc. The combined organic layers were washed with brine, dried over anhydrous Na₂SO₄. After filtration, the filtrate was concentrated under reduced pressure to afford the product intermediate compound 2.

Into a round-bottom flask were added intermediate compound 1 (1 equiv), DMF, Amino Acid Phenylalanine (1.5 equiv), DIEA (5 equiv) and HATU (1.5 equiv) at room temperature. The resulting mixture was stirred for 1 h at room temperature. The resulting mixture was extracted with EtOAc. The combined organic layers were washed with brine, dried over anhydrous Na₂SO₄. After filtration, the filtrate was concentrated under reduced pressure. The residue was purified by Prep-TLC (PE/EA 1:1) to afford the product intermediate compound 3.

Into a round-bottom flask were added intermediate compound 3, DCM/TFA (3/1) at room temperature. The resulting mixture was stirred for 2 h at room temperature. The resulting mixture was concentrated under reduced pressure. The crude product was purified by Prep-HPLC to afford the final product Compound 4. Compounds 5-12 were synthesized according to Scheme 3 by substituting the nitrobenzoic acid with the corresponding nitrobenzoic acids and phenylalanine with corresponding amino acids to produce intermediate compounds 1 and 3, respectively. Structures of the compounds 4-12 are listed in Table 1.

Compound 4: MS (ESI) (m/z) [M+H]⁺ calculated: 677.3; found: 677.3. Compound 5: MS (ESI) (m/z): [M+1]⁺ calculated: 677.3; found: 677.3. Compound 6: MS (ESI) (m/z) [M+H]⁺ calculated: 629.3; found: 629.5. Compound 7: MS (ESI) (m/z) [M+H]⁺ calculated: 677.3; found: 677.3. Compound 8: MS (ESI) (m/z) [M+H]⁺ calculated: 677.3; found: 677.2. Compound 9: MS (ESI) (m/z): [M+1]⁺ calculated: 641.3; found: 641.2. Compound 10: MS (ESI) (m/z): [M+1]⁺ calculated: 689.3; found: 689.2.

### Example 5: Synthesis of Compounds 11-12

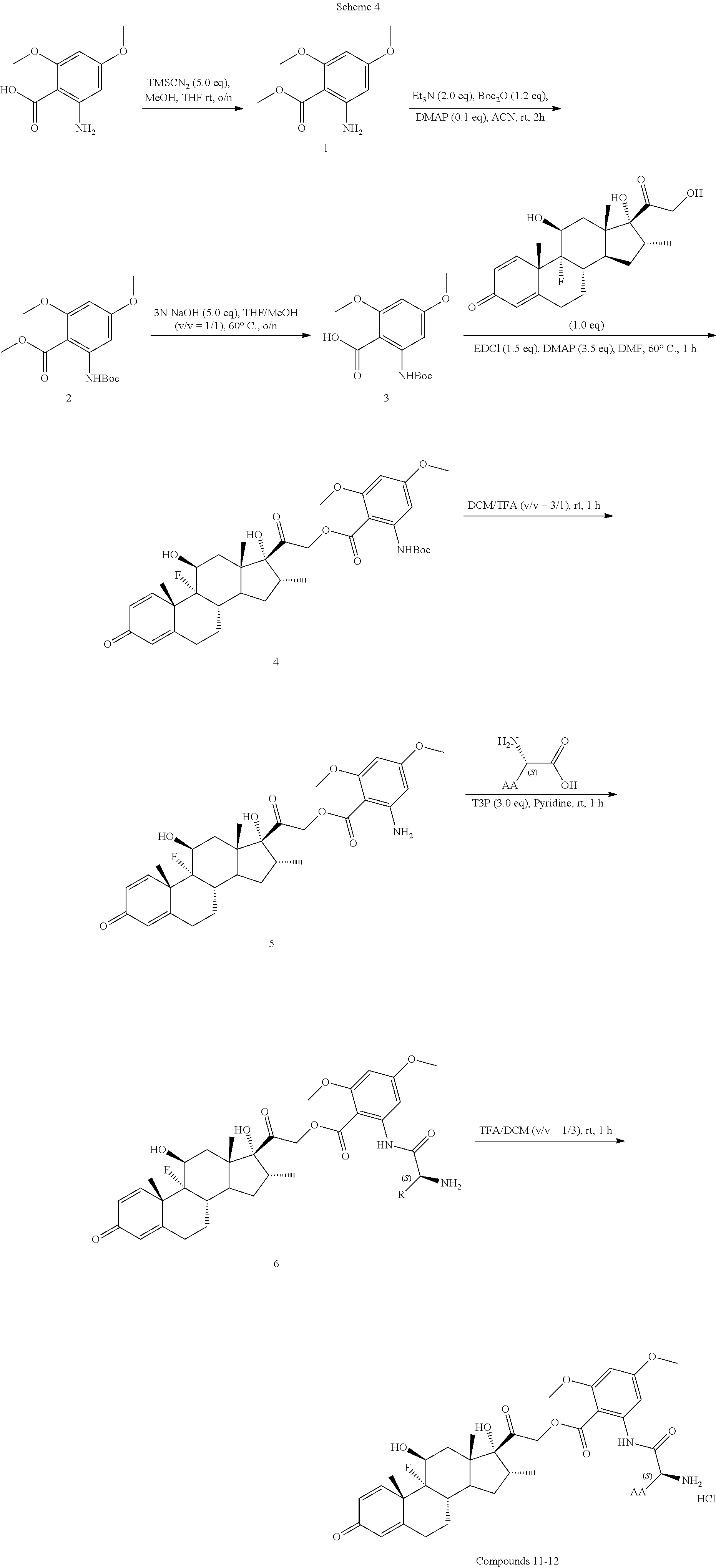

Into a 100 mL round-bottom flask were added 2-amino-4,6-dimethoxybenzoic acid (500 mg, 2.53 mmol, 1 equiv), McOH (5 mL), THF (5 mL) and TMSCHN2 (6.34 mL, 12.68 mmol, 5 equiv) at room temperature. The resulting mixture was stirred overnight at room temperature. The reaction was quenched with sat. NH4Cl (aq.) at room temperature. The resulting mixture was extracted with EtOAc (2×20 mL). The combined organic layers were washed with brine (2×30 mL), dried over anhydrous Na₂SO₄. After filtration, the filtrate was concentrated under reduced pressure. This resulted in methyl 2-amino-4,6-dimethoxybenzoate (intermediate compound 1) (420 mg, 78.42%) as a white solid.

Into a 50 mL round-bottom flask were added intermediate compound 1 (1 g, 4.73 mmol, 1 equiv), ACN (10 mL), Et₃N (1.32 mL, 9.46 mmol, 2 equiv), Boc2O (1.24 g, 5.68 mmol, 1.2 equiv) and DMAP (57.84 mg, 0.47 mmol, 0.1 equiv) at room temperature. The resulting mixture was stirred for 2 h at room temperature. The resulting mixture was diluted with water (30 mL). The resulting mixture was extracted with EtOAc (50 mL). The residue was washed with brine (50 mL). The combined organic layers were dried over anhydrous Na₂SO₄. After filtration, the filtrate was concentrated under reduced pressure. The residue was purified by silica gel column chromatography, eluted with PE/EA (68:32) to afford intermediate compound 2 (820 mg, 55.63%) as an off-white solid.

Into a 40 mL vial were added intermediate compound 2 (400 mg, 1.28 mmol, 1 equiv), THF (2 mL), MeOH (2 mL) and 3N NaOH (2.14 mL, 6.42 mmol, 5 equiv) at room temperature. The resulting mixture was stirred overnight at 60° C. The mixture was acidified to pH 4 with HCl (aq.). The resulting mixture was extracted with EtOAc (50 mL). The combined organic layers were washed with brine (60 mL), dried over anhydrous Na₂SO₄. After filtration, the filtrate was concentrated under reduced pressure. This resulted in intermediate compound 3 (370 mg, 96.86%) as a light yellow solid.

Into a round-bottom flask were added intermediate compound 3 (1 equiv), DMF, DCM, dexamethasone (1 equiv) and DMAP (0.5 equiv) at room temperature. Then were added EDCI (1.5 equiv) at room temperature. The resulting mixture was stirred for 1 h at 60° C. The resulting mixture was concentrated under reduced pressure. The residue was purified by Prep-TLC (PE/EA 1:2) to afford the intermediate compound 4. Into a round-bottom flask were added intermediate compound 4, DCM/TFA (3/1) at room temperature. The resulting mixture was stirred for 1 h at room temperature. The resulting mixture was concentrated under reduced pressure. The crude product intermediate compound 5 was used in the next step directly without further purification.

Into a round-bottom flask were added intermediate compound 5 (1 equiv), Py (s), Amino acids (3 equiv) and T3P (3 equiv, 50% in EA) at room temperature. The resulting mixture was stirred for 1 h at room temperature. The resulting mixture was extracted with EtOAc. The combined organic layers were washed with brine, dried over anhydrous Na₂SO₄. After filtration, the filtrate was concentrated under reduced pressure. The residue was purified by Prep-TLC (PE/EA 1:2) to afford the crude product intermediate compound 6. The crude product was purified by Prep-HPLC

Into a round-bottom flask were added intermediate compound 6, DCM/TFA (3/1) at room temperature. The resulting mixture was stirred for 1 h at room temperature. The resulting mixture was concentrated under reduced pressure. The crude product was purified by Prep-HPLC with the following conditions to afford the final product Compounds 11-12. Compound 10: MS (ESI) (m/z) [M+H]⁺ calculated: 707.2; found: 707.2. Compound 11: MS (ESI) (m/z) [M+H]⁺ calculated: 755.2; found: 755.2.

### Example 6: Synthesis of Compound 13

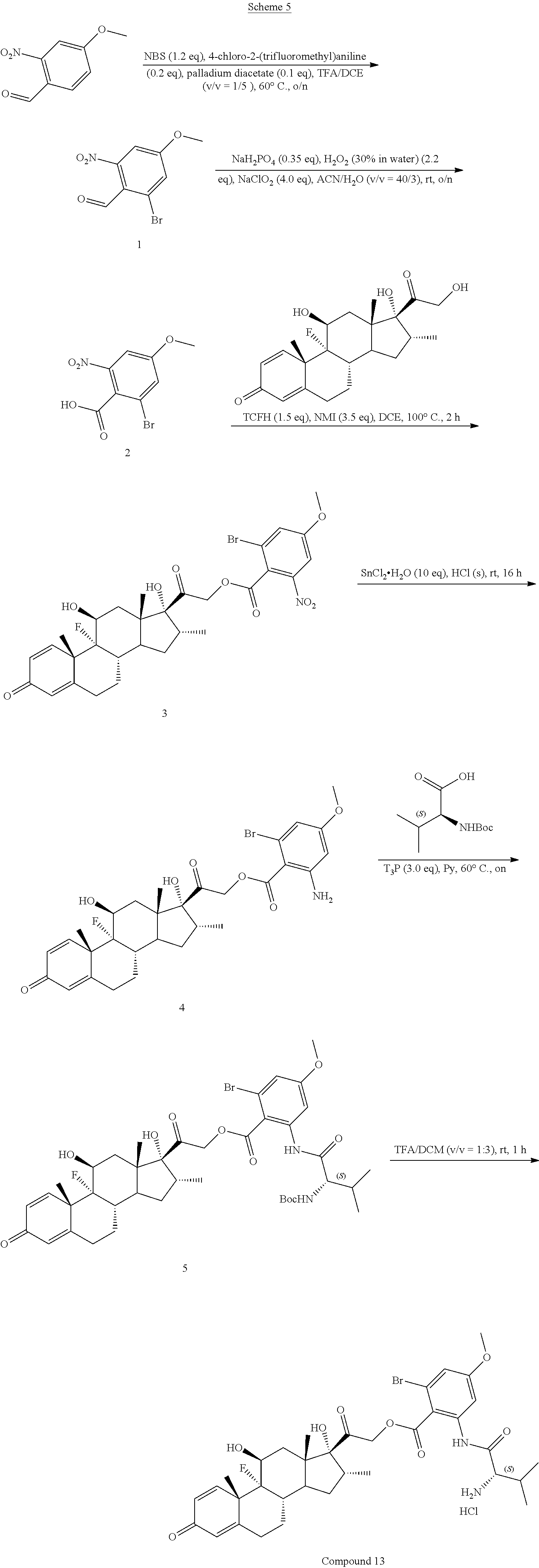

To a stirred solution of 4-methoxy-2-nitrobenzaldehyde (1 g, 5.52 mmol, 1 equiv) and NBS (1.18 g, 6.62 mmol, 1.2 equiv) in DCE (25 mL) and TFA (5 mL) were added 4-chloro-2-(trifluoromethyl) aniline (0.22 g, 1.10 mmol, 0.2 equiv) and Pd(OAc)₂ (0.12 g, 0.55 mmol, 0.1 equiv) at room temperature under nitrogen atmosphere. The resulting mixture was stirred overnight at 60° C. under nitrogen atmosphere. The resulting mixture was diluted with ethyl acetate (20 mL) and water (20 mL). The resulting mixture was extracted with EtOAc (2×30 mL). The combined organic layers were washed with brine (3×30 mL), dried over anhydrous Na₂SO₄. After filtration, the filtrate was concentrated under reduced pressure. The residue was purified by silica gel column chromatography, eluted with PE/EA (1:1) to afford intermediate compound 1 (1.2 g, 83.59%) as a white solid.

To a stirred solution of intermediate compound 1 (500 mg, 1.92 mmol, 1 equiv) and NaH₂PO₄ (0.08 g, 0.67 mmol, 0.35 equiv) in ACN (20 mL) and H₂O (1.5 mL) were added H₂O₂ (30%) (0.33 mL, 4.23 mmol, 2.2 equiv) and NaClO2 (0.70 g, 7.69 mmol, 4 equiv) at room temperature. The resulting mixture was stirred overnight at room temperature. The mixture was acidified to pH 3 with 1N HCl. The resulting mixture was extracted with EtOAc (3×30 mL). The combined organic layers were washed with brine (2×50 mL), dried over anhydrous Na₂SO₄. After filtration, the filtrate was concentrated under reduced pressure. This resulted in intermediate compound 2 (500 mg, 94.20%) as a brown oil. (220 mg, 96.41%).

To a stirred solution of intermediate compound 2 (500 mg, 1.81 mmol, 1 equiv) and dexamethasone (710.88 mg, 1.81 mmol, 1 equiv) in DCE (12 mL) were added TCFH (762.33 mg, 2.71 mmol, 1.5 equiv) and NMI (520.52 mg, 6.33 mmol, 3.5 equiv) at room temperature. The resulting mixture was stirred 2 h at 100° C. The resulting mixture was diluted with ethyl acetate (20 mL) and water (20 mL). The resulting mixture was extracted with EtOAc (2×20 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EA (1:2) to afford intermediate compound 3 (700 mg, 59.41%) as a white solid.

To a stirred mixture of intermediate compound 3 (680 mg, 1.04 mmol, 1 equiv) in HCl (5 mL) were added SnCl₂·2H₂O (2.38 g, 10.45 mmol, 10 equiv) at room temperature. The resulting mixture was stirred for 16 h at room temperature. The mixture was neutralized to pH 8 with saturated NaHCO₃. The resulting mixture was extracted with EtOAc (3×100 mL). The combined organic layers were washed with brine (2×300 mL), dried over anhydrous Na₂SO₄. After filtration, the filtrate was concentrated under reduced pressure. This resulted in intermediate compound 4 (680 mg, 94.35%) as a white solid.

To a stirred solution of intermediate compound 4 (1 equiv) and amino acid (2 equiv) in Pyridine were added T3P (3 equiv, 50%) at room temperature. The resulting mixture was stirred overnight at 60° C. The resulting mixture was concentrated under reduced pressure. The residue was purified by silica gel column chromatography, eluted with PE/EA (1:1) to afford intermediate compound 5.

Into a round-bottom flask were added intermediate compound 5, DCM/TFA (3/1) at room temperature. The resulting mixture was stirred for 1 h at room temperature. The resulting mixture was concentrated under reduced pressure. The crude product was purified by Prep-HPLC to afford compound 13. MS (ESI) (m/z) [M+H]⁺ calculated: 719.2; found: 719.2.

### Example 7: Synthesis of Compound 14

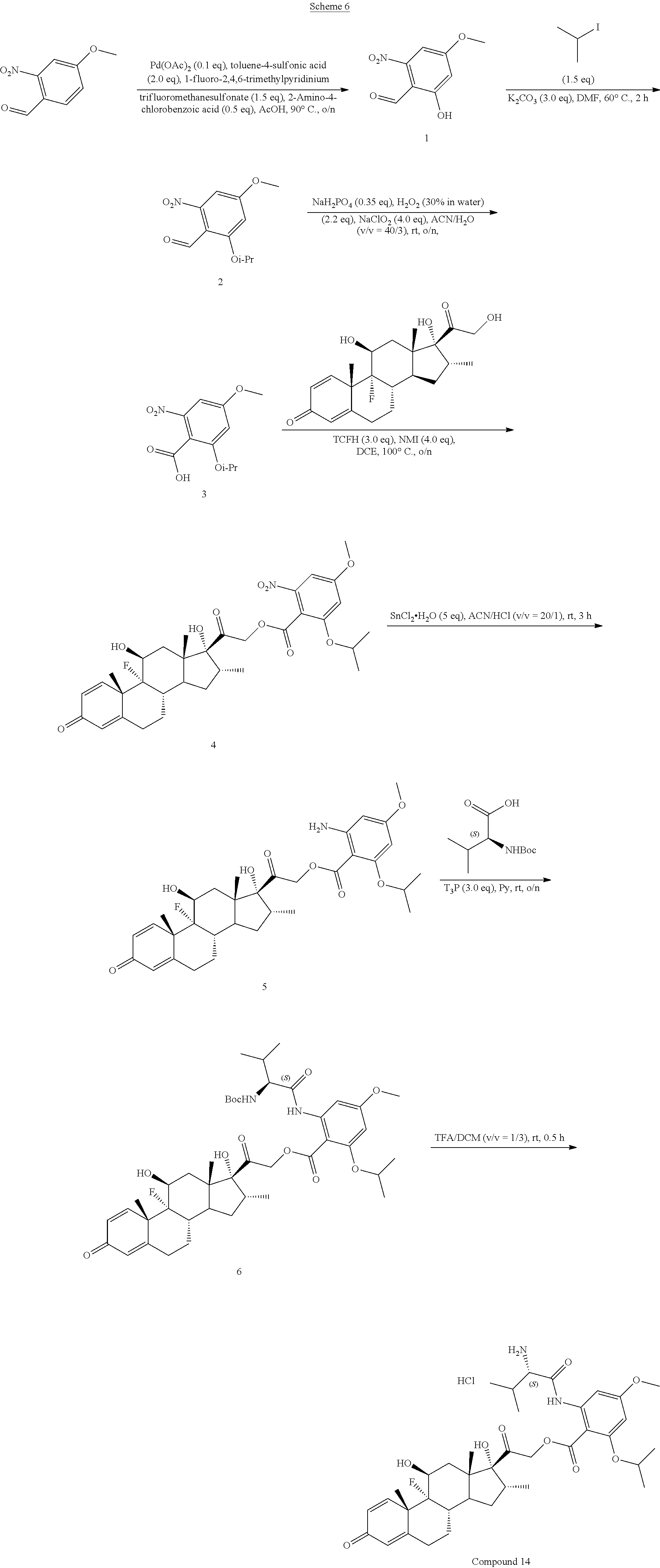

To a stirred solution of 4-methoxy-2-nitrobenzaldehyde (1 g, 5.52 mmol, 1 equiv) in AcOH (5 mL) was added Pd(OAc) 2 (123.94 mg, 0.55 mmol, 0.1 equiv), 2-amino-4-chlorobenzoic acid (0.47 g, 2.76 mmol, 0.5 equiv), 1-fluoro-2,4,6-trimethylpyridinium trifluoromethanesulfonate (2.40 g, 8.28 mmol, 1.5 equiv), p-toluenesulfonic acid (1.90 g, 11.04 mmol, 2 equiv) at room temperature under nitrogen atmosphere. The resulting mixture was stirred overnight at 90° C. The resulting mixture was filtered, the filter cake was washed with DCM (2×20 mL). The filtrate was concentrated under reduced pressure. The residue was purified by silica gel column chromatography, eluted with PE/EA (10:1) to afford intermediate compound 1 (380 mg, 34.92%) as a yellow solid.

To a stirred solution of intermediate compound 1 (380 mg, 1.92 mmol, 1 equiv) and 2-iodopropane (491.49 mg, 2.89 mmol, 1.5 equiv) in DMF (4 mL) was added K2CO3 (799.17 mg, 5.78 mmol, 3 equiv) at room temperature. The resulting mixture was stirred 2 h at 60° C. The resulting mixture was diluted with EtOAc (20 mL) and water (20 mL). The resulting mixture was extracted with EtOAc (2×50 mL). The combined organic layers were washed with brine (2×100 mL), dried over anhydrous Na₂SO₄. After filtration, the filtrate was concentrated under reduced pressure. The residue was purified by silica gel column chromatography, eluted with PE/EA (3:1) to afford intermediate compound 2 (380 mg, 82.41%) as a brown solid.

To a stirred solution of intermediate compound 2 (400 mg, 1.67 mmol, 1 equiv) and NaH2PO4 (70.21 mg, 0.58 mmol, 0.35 equiv) in ACN (8 mL) and H₂O (0.6 mL) was added H₂O₂ (125.12 mg, 3.67 mmol, 2.2 equiv, 30%) and NaClO2 (604.88 mg, 6.68 mmol, 4 equiv) at room temperature. The resulting mixture was stirred overnight at room temperature. The mixture was acidified to pH 4 with 1N HCl. The resulting mixture was extracted with EtOAc (2×70 mL). The combined organic layers were washed with brine (2×100 mL), dried over anhydrous Na₂SO₄. After filtration, the filtrate was concentrated under reduced pressure. This resulted in intermediate compound 3 (340 mg, 79.67%) as a green solid.

To a stirred solution of intermediate compound 3 (160 mg, 0.62 mmol, 1 equiv) and dexamethasone (246.04 mg, 0.62 mmol, 1 equiv) in DCE (2 mL) were added TCFH (527.68 mg, 1.88 mmol, 3 equiv) and NMI (205.89 mg, 2.51 mmol, 4 equiv) at room temperature. The resulting mixture was stirred overnight at 100° C. The resulting mixture was diluted with ethyl acetate (20 mL) and water (20 mL). The resulting mixture was extracted with EtOAc (2×20 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EA (1:2) to afford intermediate compound 4 (120 mg, 30.40%) as a white solid.

To a stirred mixture of intermediate compound 4 (140 mg, 0.22 mmol, 1 equiv) in ACN (2.8 mL) and HCl (0.14 mL) were added SnCl₂·2H₂O (253.08 mg, 1.110 mmol, 5 equiv) at room temperature. The resulting mixture was stirred for 3 h at room temperature. The mixture was neutralized to pH 8 with saturated NaHCO₃. The resulting mixture was extracted with EtOAc (3×100 mL). The combined organic layers were washed with brine (2×300 mL), dried over anhydrous Na₂SO₄. After filtration, the filtrate was concentrated under reduced pressure. This resulted in intermediate compound 5 (110 mg, 82.50%) as a white solid.

To a stirred solution of intermediate compound 5 (1 equiv) and amino acid (2 equiv) in Pyridine were added T3P (3 equiv, 50%) at room temperature. The resulting mixture was stirred overnight at room temperature. The resulting mixture was concentrated under reduced pressure. The residue was purified by silica gel column chromatography, eluted with PE/EA (1:1) to afford the product intermediate compound 6. Into a round-bottom flask were added intermediate compound 6, DCM/TFA (3/1) at room temperature. The resulting mixture was stirred for 0.5 h at room temperature. The resulting mixture was concentrated under reduced pressure. The crude product was purified by Prep-HPLC to afford the compound 14. MS (ESI) (m/z) [M+H]⁺ calculated: 699.4; found: 699.4.

### Example 8: Synthesis of Compound 15

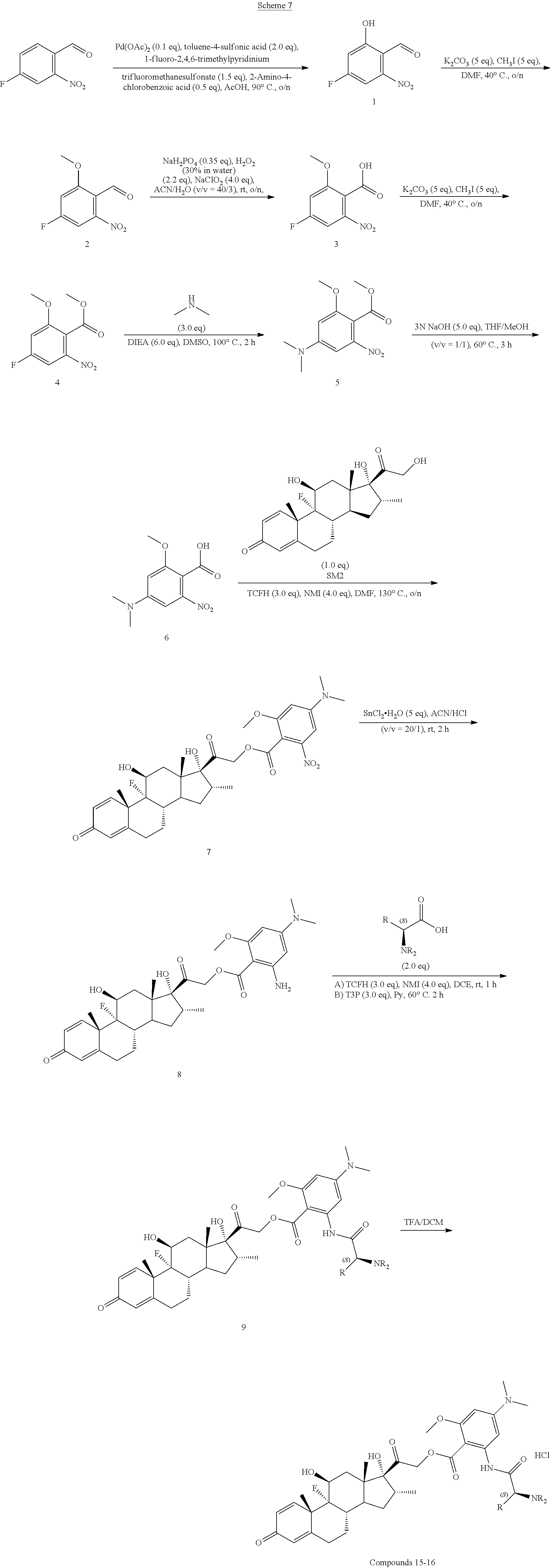

To a stirred solution of 4-fluoro-2-nitrobenzaldehyde (2 g, 11.82 mmol, 1 equiv) and 2-amino-4-chlorobenzoic acid (1.01 g, 5.91 mmol, 0.5 equiv) in ACOH (5 mL) were added Pd(AcO)₂ (0.27 g, 1.18 mmol, 0.1 equiv), fluoro-2,4,6-trimethylpyridinium triflate (5.13 g, 17.74 mmol, 1.5 equiv) and p-toluenesulfonic acid (4.07 g, 23.65 mmol, 2 equiv) at room temperature. The resulting mixture was stirred overnight at 90° C. The resulting mixture was diluted with EtOAc (50 mL) and water (50 mL). The resulting mixture was extracted with EtOAc (2×50 mL). The combined organic layers were washed with brine (2×100 mL), dried over anhydrous Na₂SO₄. After filtration, the filtrate was concentrated under reduced pressure. The residue was purified by silica gel column chromatography, eluted with PE/EA (10:1) to afford intermediate compound 1 (700 mg, 31.97%) as a yellow solid.

To a stirred solution of intermediate compound 1 (700 mg, 3.78 mmol, 1 equiv) and K₂CO₃ (2.61 g, 18.91 mmol, 5 equiv) in DMF (7 mL) was added CH₃I (1.18 mL, 18.91 mmol, 5 equiv) at room temperature. The resulting mixture was stirred overnight at 40° C. The resulting mixture was diluted with EtOAc (20 mL) and water (20 mL). The resulting mixture was extracted with EtOAc (2×30 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EA (5:1) to afford intermediate compound 2 (550 mg, 73.04%) as a yellow solid.

To a stirred solution of intermediate compound 2 (550 mg, 2.76 mmol, 1 equiv) and NaH₂PO₄ (115.98 mg, 0.96 mmol, 0.35 equiv) in ACN (4 mL) and H₂O (0.3 mL) were added H₂O₂ (30%) (0.47 mL, 6.07 mmol, 2.2 equiv) and NaClO2 (999.15 mg, 11.04 mmol, 4 equiv) at room temperature. The resulting mixture was stirred overnight at room temperature. The mixture was acidified to pH 5 with 1N HCl. The resulting mixture was extracted with EtOAc (2×20 mL). The combined organic layers were washed with brine (2×30 mL), dried over anhydrous Na₂SO₄. After filtration, the filtrate was concentrated under reduced pressure. This resulted in intermediate compound 3 (530 mg, 89.20%) as a yellow solid.

To a stirred solution of intermediate compound 3 (530 mg, 2.46 mmol, 1 equiv) and K₂CO₃ (1.70 g, 12.32 mmol, 5 equiv) in DMF (5 mL) was added CH3I (0.77 mL, 12.32 mmol, 5 equiv) at room temperature. The resulting mixture was stirred overnight at 40° C. The resulting mixture was diluted with EtOAc (10 mL) and water (10 mL). The resulting mixture was extracted with EtOAc (2×20 mL). The combined organic layers were washed with brine (2×30 mL), dried over anhydrous Na₂SO₄. After filtration, the filtrate was concentrated under reduced pressure. The residue was purified by Prep-TLC (PE/EA 5:1) to afford intermediate compound 4 (500 mg, 88.57%) as a yellow solid.

To a stirred solution of intermediate compound 4 (500 mg, 2.18 mmol, 1 equiv) and dimethylamine (295.11 mg, 6.54 mmol, 3.00 equiv) in DMSO (5 mL) was added DIEA (1691.99 mg, 13.09 mmol, 6 equiv) at room temperature. The resulting mixture was stirred for 2 h at 100° C. The resulting mixture was diluted with water (30 mL). The resulting mixture was extracted with EtOAc (50 mL). The residue was washed with brine (50 mL). The combined organic layers were dried over anhydrous Na₂SO₄. After filtration, the filtrate was concentrated under reduced pressure. This resulted in intermediate compound 5 (500 mg, 90.14%) as a yellow solid.

To a stirred solution of intermediate compound 5 (200 mg, 0.78 mmol, 1 equiv) in THF (1 mL) and MeOH (1 mL) were added 3N NaOH (1.3 mL, 3.93 mmol, 5 equiv) at room temperature. The resulting mixture was stirred for 3 h at 60° C. The mixture was acidified to pH 6 with 1N HCl. The resulting mixture was extracted with EtOAc (30 mL). The combined organic layers were washed with brine (50 mL), dried over anhydrous Na₂SO₄. After filtration, the filtrate was concentrated under reduced pressure. This resulted intermediate compound 6 (180 mg, 95.26%) as a yellow solid. MS (ESI) (m/z): [M+1]⁺ calculated: 241.1; found: 241.1.

Into a round-bottom flask were added intermediate compound 6 (1 equiv), DMF, dexamethasone (1 equiv), TCFH (3.0 equiv) and NMI (4.0 equiv) at room temperature. The resulting mixture was stirred overnight at 130° C. The resulting mixture was extracted with EtOAc. The combined organic layers were washed with brine, dried over anhydrous Na₂SO₄. After filtration, the filtrate was concentrated under reduced pressure. The residue was purified by Prep-TLC (PE/EA 1:2) to afford the product. intermediate compound 7

Into a round-bottom flask were added intermediate compound 7 (1 equiv), SnCl₂·2H₂O (5 equiv) and ACN/HCl (v/v=20/1) at room temperature. The resulting mixture was stirred for 2 h at room temperature. The mixture was basified to pH 8 with saturated NaHCO₃ (aq.). The resulting mixture was extracted with EtOAc. The combined organic layers were washed with brine, dried over anhydrous Na₂SO₄. After filtration, the filtrate was concentrated under reduced pressure. The crude product intermediate compound 8 was used in the next step directly without further purification.

Into a round-bottom flask were added intermediate compound 8 (1 equiv), Py(s), Amino acids (3 equiv) and T3P (3 equiv, 50% in EA) at room temperature. The resulting mixture was stirred for 2 h at 60° C. The resulting mixture was extracted with EtOAc. The combined organic layers were washed with brine, dried over anhydrous Na₂SO₄. After filtration, the filtrate was concentrated under reduced pressure. The residue was purified by silica gel column chromatography, eluted with PE/EA (100%˜70%, 45 min) to afford the product intermediate compound 9.

Into a round-bottom flask were added intermediate compound 9, DCM/TFA (3/1) at room temperature. The resulting mixture was stirred for 2 h at room temperature. The resulting mixture was concentrated under reduced pressure. The crude product was purified by Prep-HPLC to afford obtained the final product compound 15. MS (ESI) (m/z) [M+H]⁺ calculated: 768.3; found: 768.3.

### Example 9: Synthesis of Compound 16

Into a round-bottom flask were added intermediate compound 8 of Example 8 (1 equiv), DCE, Amino acids (2 equiv), TCFH (3.0 equiv) and NMI (4.0 equiv) at room temperature. The resulting mixture was stirred for 1 h at room temperature. The resulting mixture was extracted with EtOAc. The combined organic layers were washed with brine, dried over anhydrous Na₂SO₄. After filtration, the filtrate was concentrated under reduced pressure. The residue was purified by Prep-TLC (PE/EA 1:2) to afford the crude product. The crude product was purified by Prep-HPLC to afford the final product compound 16. MS (ESI) (m/z) [M+H]⁺ calculated: 759.9; found: 759.9.

### Example 10: Synthesis of Compound 17

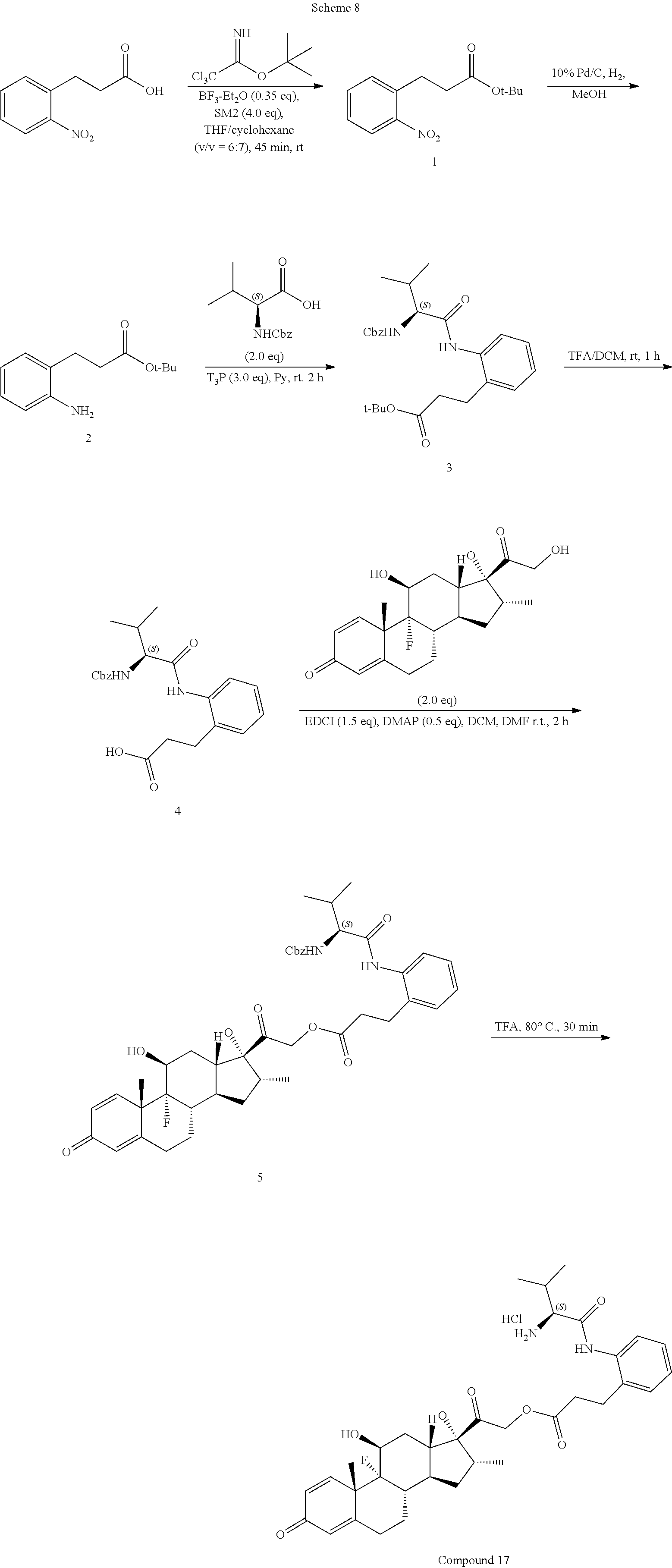

To a stirred solution of 3-(2-nitrophenyl) propanoic acid (2 g, 10.247 mmol, 1.0 equiv) in THF (30 mL) and cyclohexane (35 mL) was added tert-butyl 2,2,2-trichloroethanimidate (8.96 g, 40.988 mmol, 4.0 equiv) and BF3·Et2O (0.51 g, 3.586 mmol, 0.35 equiv) dropwise at room temperature. The resulting mixture was stirred for 45 min at room temperature under nitrogen atmosphere. The resulting mixture was extracted with EtOAc (200× mL). The combined organic layers were washed with brine (3×200 mL), dried over anhydrous Na₂SO₄. After filtration, the filtrate was concentrated under reduced pressure. The residue was purified by silica gel column chromatography, eluted with PE/EA (4:1) to afford intermediate compound 1 (2.4 g, 91.34%) as an off-white oil.

To a stirred solution of intermediate compound 1 (900 mg, 3.582 mmol, 1.0 equiv) in methanol (12 mL) was added Pd/C (900 mg, 0.423 mmol, 0.12 equiv, 5%) at room temperature. The resulting mixture was stirred for 2 h at room temperature under hydrogen atmosphere. The resulting mixture was filtered, the filter cake was washed with EtOAc (3×5 mL). The filtrate was concentrated under reduced pressure to afford intermediate compound 2 (800 mg, 72.67%) as a light yellow oil.

To a stirred solution of intermediate compound 2 (1.0 equiv) and Amino acids (2.0 equiv) in Pyridine was added T3P (3.00 equiv, 50%) at room temperature. The resulting mixture was stirred for 2 h at room temperature. The resulting mixture was extracted with EtOAc. The combined organic layers were washed with brine, dried over anhydrous Na₂SO₄. After filtration, the filtrate was concentrated under reduced pressure. The residue was purified by silica gel column chromatography, eluted with PE/EA (100%˜70%, 45 min) to afford intermediate compound 3

Into a round-bottom flask were added intermediate compound 3, DCM/TFA (3/1) at room temperature. The resulting mixture was stirred for 1 h at room temperature. The resulting mixture was concentrated under reduced pressure. The crude product intermediate compound 4 was used in the next step directly without further purification.

To a stirred solution of intermediate compound 4 (1.00 equiv) and dexamethasone (2.00 equiv) in DCM and DMF was added EDCI (1.50 equiv) DMAP (0.50 equiv) in portions at room temperature. The resulting mixture was stirred for 2 h at room temperature under nitrogen atmosphere. The resulting mixture was concentrated under reduced pressure. The residue was purified by reverse flash chromatography with the following conditions: column, C₁₈ silica gel; mobile phase, MeCN in Water (0.1% TFA), 10% to 50% gradient in 10 min; detector, UV 254 nm to afford intermediate compound 5.

Into a 20 mL vial were added intermediate compound 5 (1 equiv) and TFA(s) at room temperature. The resulting mixture was stirred for 30 min at 80° C. The resulting mixture was concentrated under reduced pressure. The crude product was purified by Prep-HPLC to afford compound 17. MS (ESI) (m/z) [M+H]⁺ calculated: 639.3; found: 639.3.

### Example 11: Synthesis of Compound 18

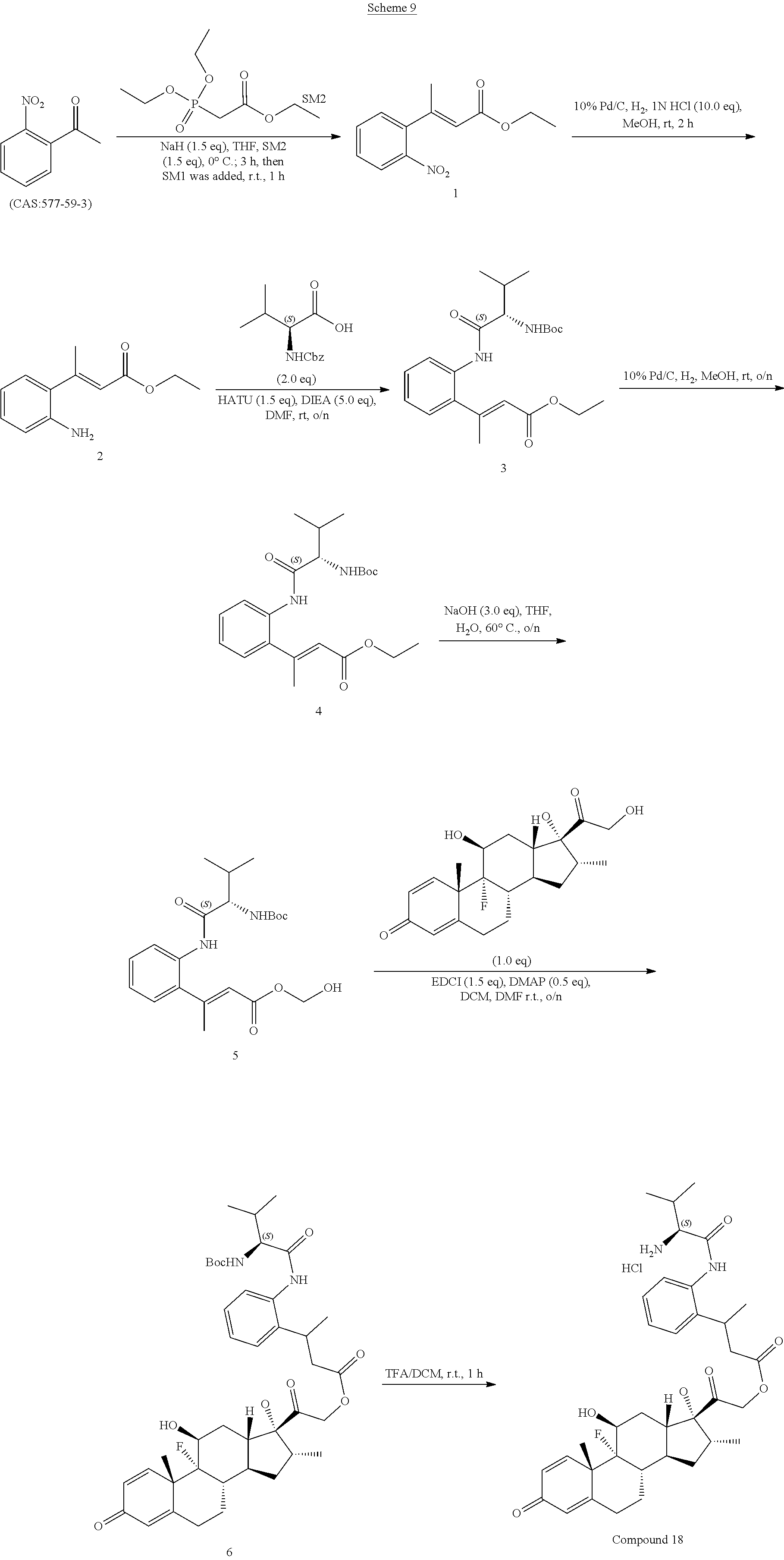

To a solution of triethyl phosphonoacetate (4.07 g, 18.165 mmol, 1.5 equiv) in THF (10 mL) was added NaH (0.73 g, 18.165 mmol, 1.5 equiv, 60%). The mixture was stirred for 3 h. 2-nitroacetophenone (2 g, 12.110 mmol, 1 equiv) in THF (10 mL) was added and the mixture was allowed to warm to RT and stirred for 1 h. The reaction mixture was quenched by water and extracted with EtOAc (3×50 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EA (2:1) to afford intermediate compound 1 (1.2 g, 42.12%) as a white solid. MS (ESI) (m/z): [M+1]+ calculated: 236.1; found: 236.2.

Into a 100 mL round-bottom flask were added ethyl (intermediate compound 1 (400 mg, 1.700 mmol, 1 equiv), HCl (4 mL) and SnCl₂·2H₂O (1.94 g, 8.500 mmol, 5 equiv) at room temperature. The resulting mixture was stirred for 2 days at 30° C. The resulting mixture was concentrated under reduced pressure. The resulting mixture was diluted with EtOAc (50 mL). The mixture was basified to pH 8 with saturated NaHCO₃ (aq.). The resulting mixture was extracted with EtOAc (2×50 mL). The combined organic layers were washed with brine (2×100 mL), dried over anhydrous Na₂SO₄. After filtration, the filtrate was concentrated under reduced pressure. This resulted in intermediate compound 2 (400 mg, 45.84%) as a white crude solid. MS (ESI) (m/z): [M+1]+ calculated: 206.1; found: 206.2.

Into a 50 mL round-bottom flask were added intermediate compound 2 (400 mg, 0.780 mmol, 1 equiv), DMF (8 mL), (2S)-2-[(tert-butoxycarbonyl)amino]-3-methylbutanoic acid (338.72 mg, 1.560 mmol, 2 equiv), DIEA (503.75 mg, 3.900 mmol, 5 equiv) and HATU (889.19 mg, 2.340 mmol, 3 equiv) at room temperature. The resulting mixture was stirred overnight at 30° C. The resulting mixture was extracted with EtOAc (2×30 mL). The combined organic layers were washed with brine (3×50 mL), dried over anhydrous Na₂SO₄. After filtration, the filtrate was concentrated under reduced pressure. The residue was purified by silica gel column chromatography, eluted with PE/EA (1:1) to afford intermediate compound 3 (200 mg, 63.43%) as a light yellow solid. MS (ESI) (m/z): [M+1]+ calculated: 405.2; found: 405.2.

To a solution of intermediate compound 3 (200 mg, 0.494 mmol, 1 equiv) in 8 mL MeOH was added Pd/C (10%, 0.1 g) in a 50 mL round-bottom flask. The mixture was hydrogenated at room temperature overnight under hydrogen atmosphere using a hydrogen balloon, filter through a Celite pad and concentrated under reduced pressure. This resulted in intermediate compound 4 as a white solid. MS (ESI) (m/z): [M+1]+ calculated: 407.2; found: 407.3.

Into a 50 mL round-bottom flask were added intermediate compound 4 (200 mg, 0.492 mmol, 1 equiv), THF (2 mL) and NaOH (0.49 mL, 1.476 mmol, 3 equiv) at room temperature. The resulting mixture was stirred overnight at 60° C. The mixture was acidified to pH 5 with 1N HCl (aq.). The resulting mixture was extracted with EtOAc (2×50 mL). The combined organic layers were washed with brine (2×50 mL), dried over anhydrous Na₂SO₄. After filtration, the filtrate was concentrated under reduced pressure. This resulted in intermediate compound 5 as a white solid. MS (ESI) (m/z): [M+1]+ calculated: 379.2; found: 379.3.

Into a 50 mL round-bottom flask were added intermediate compound 5 (200 mg, 0.528 mmol, 1 equiv), DMF (2 mL), DCM (2 mL), dexamethasone (207.40 mg, 0.528 mmol, 1 equiv), EDCI (151.95 mg, 0.792 mmol, 1.5 equiv) and DMAP (32.28 mg, 0.264 mmol, 0.5 equiv) at room temperature. The resulting mixture was stirred for 2 h at 30° C. The resulting mixture was extracted with EtOAc (2×30 mL). The combined organic layers were washed with brine (3×50 mL), dried over anhydrous Na₂SO₄. After filtration, the filtrate was concentrated under reduced pressure. The residue was purified by silica gel column chromatography, eluted with PE/EA (1:2) to afford intermediate compound 6 (150 mg, 37.70%) as a white solid. MS (ESI) (m/z): [M+1]+ calculated: 753.4; found: 753.4.

Into a round-bottom flask were added intermediate compound 6, DCM/TFA (3/1) at room temperature. The resulting mixture was stirred for 2 h at room temperature. The resulting mixture was concentrated under reduced pressure. The crude product was purified by Prep-HPLC to afford the final product compound 8. MS (ESI) (m/z): [M+1]+ calculated: 653.4; found: 653.3.

### Example 12: Synthesis of Compound 19

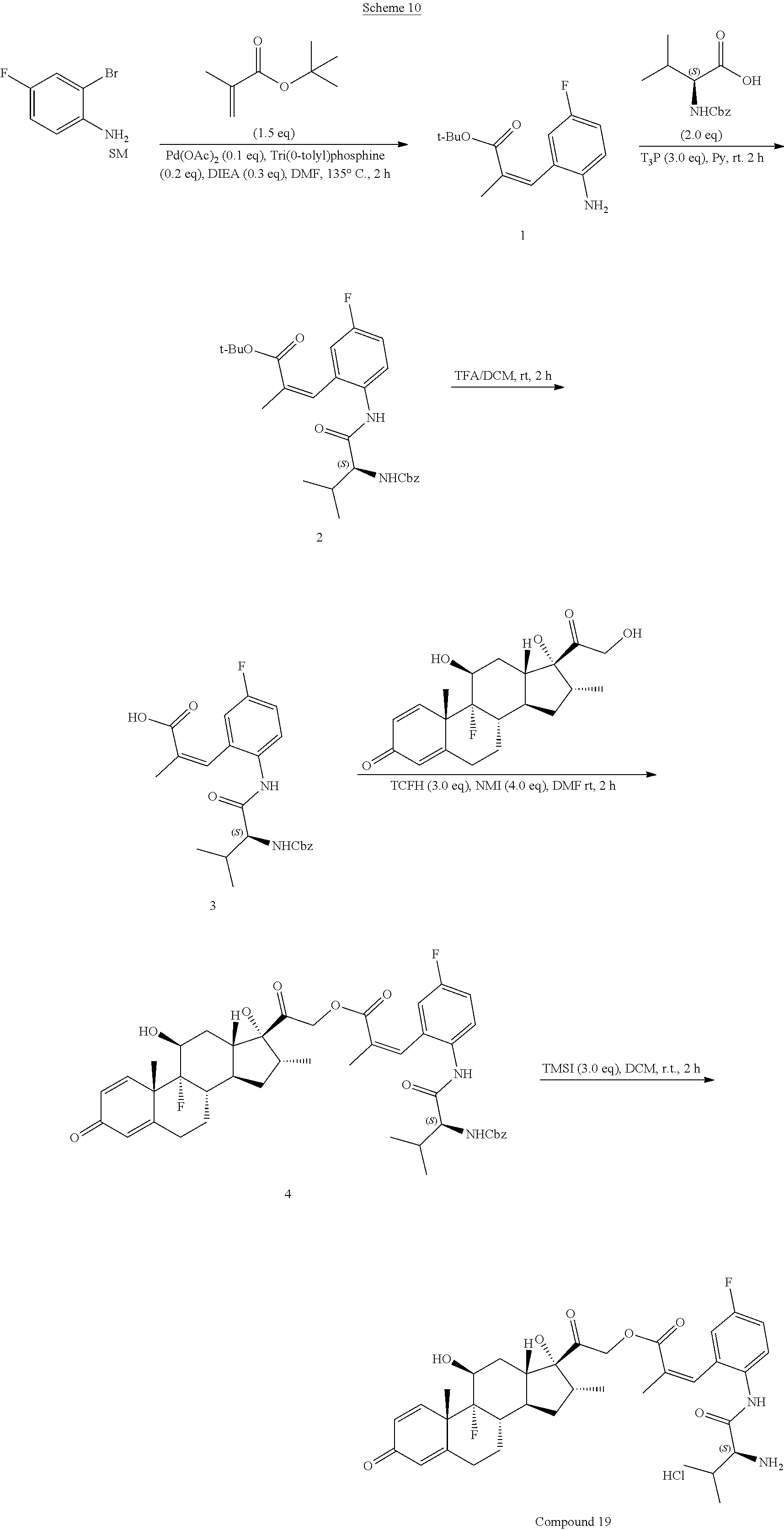

To a stirred solution of 2-bromo-4-fluoroaniline (400 mg, 2.10 mmol, 1 equiv) and tert-butyl 2-methylprop-2-enoate (299.34 mg, 2.10 mmol, 1 equiv) in DMF (5 mL) were added DIEA (816.23 mg, 6.31 mmol, 3 equiv), tris(2-methylphenyl)phosphane (128.14 mg, 0.42 mmol, 0.2 equiv) and Pd(AcO)₂ (47.26 mg, 0.21 mmol, 0.1 equiv) at room temperature. The resulting mixture was stirred for 2 h at 135° C. under nitrogen atmosphere. The resulting mixture was diluted with EtOAc (20 mL) and water (20 mL). The resulting mixture was extracted with EtOAc (2×20 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EA (1:1) to afford intermediate compound 1 (160 mg, 30.25%) as a light yellow oil.

To a stirred solution of intermediate compound 1 (160 mg, 0.63 mmol, 1 equiv) and (2S)-2-{[(benzyloxy) carbonyl]amino}-3-methylbutanoic acid (319.98 mg, 1.27 mmol, 2 equiv) in Pyridine (2 mL) was added T3P (1.22 mL, 1.91 mmol, 3 equiv, 50%) at room temperature. The resulting mixture was stirred for 2 h at room temperature. The resulting mixture was diluted with EtOAc (10 mL) and water (10 mL). The resulting mixture was extracted with EtOAc (2×10 mL). The combined organic layers were washed with brine (2×30 mL), dried over anhydrous Na₂SO₄. After filtration, the filtrate was concentrated under reduced pressure. The residue was purified by Prep-TLC (PE/EA 1:1) to afford intermediate compound 2 (200 mg, 64.83%) as a white solid.

To a stirred solution of intermediate compound 2 (200 mg, 0.41 mmol, 1 equiv) in DCM (3 mL) was added TFA (1 mL) at room temperature. The resulting mixture was stirred for 2 h at room temperature. The resulting mixture was concentrated under reduced pressure. This resulted in intermediate compound 3 (150 mg, 84.82%) as a yellow oil.

Into a round-bottom flask were added intermediate compound 3 (1 equiv), DMF, dexamethasone (1 equiv), TCFH (3.0 equiv) and NMI (4.0 equiv) at room temperature. The resulting mixture was stirred for 2 h at room temperature. The resulting mixture was extracted with EtOAc. The combined organic layers were washed with brine, dried over anhydrous Na₂SO₄. After filtration, the filtrate was concentrated under reduced pressure. The residue was purified by Prep-TLC (PE/EA 1:2) to afford intermediate compound 4.

Into a round-bottom flask were added intermediate compound 4, TMSI (3.0 equiv) and DCM at room temperature. The resulting mixture was stirred for 2 h at room temperature. The resulting mixture was concentrated under reduced pressure. The crude product was purified by Prep-HPLC to afford compound 19. MS (ESI) (m/z) [M+H]+ calculated: 705.2; found: 705.2.

### Example 13: Synthesis of Compound 20

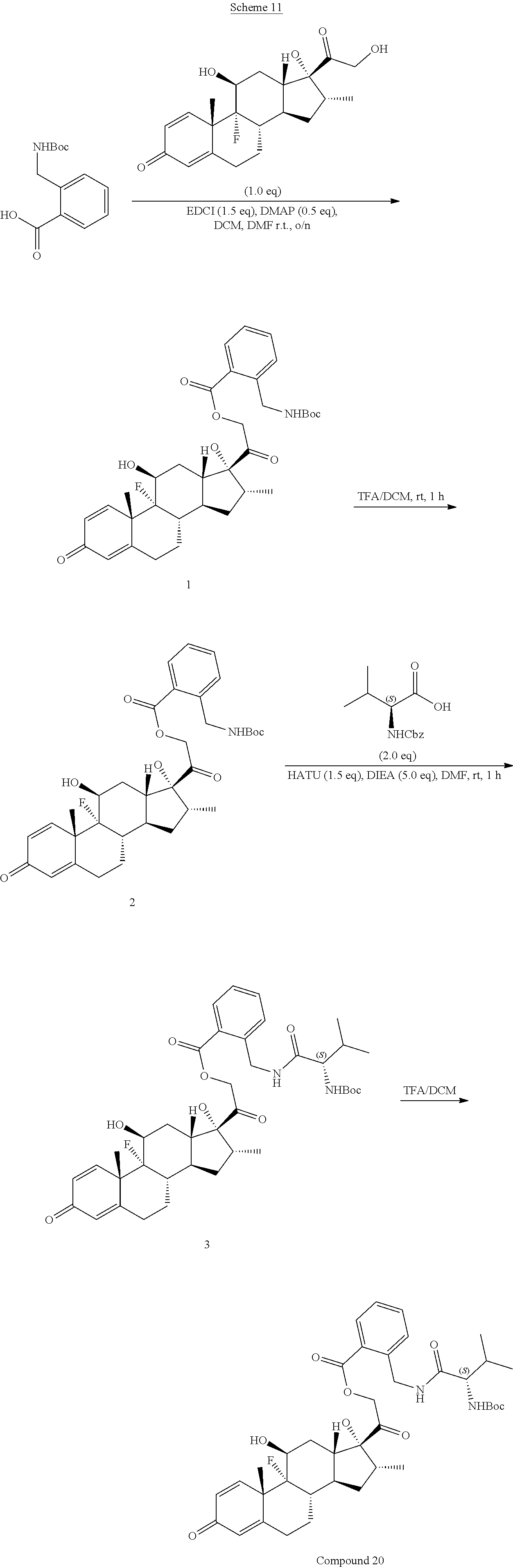

Into a round-bottom flask were added 2-{[(tert-butoxycarbonyl)amino]methyl}benzoic acid (2 equiv), DMF, DCM, dexamethasone (1 equiv) and DMAP (0.5 equiv) at room temperature. Then were added EDCI (1.5 equiv) at 0° C. The resulting mixture was stirred overnight at room temperature. The resulting mixture was concentrated under reduced pressure. The residue was purified by Prep-TLC (PE/EA 1:2) to afford intermediate compound 1. Into a round-bottom flask were added intermediate compound 1, DCM/TFA (3/1) at room temperature. The resulting mixture was stirred for 1 h at room temperature. The resulting mixture was concentrated under reduced pressure. The crude product intermediate compound 2 was used in the next step directly without further purification.

Into a round-bottom flask were added intermediate compound 2 (1 equiv), DMF, (2S)-2-[(tert-butoxycarbonyl)amino]-3-methylbutanoic acid (2.0 equiv), DIEA (5 equiv) and HATU (1.5 equiv) at room temperature. The resulting mixture was stirred for 1 h at room temperature. The resulting mixture was extracted with EtOAc. The combined organic layers were washed with brine, dried over anhydrous Na₂SO₄. After filtration, the filtrate was concentrated under reduced pressure. The residue was purified by Prep-TLC (PE/EA 1:1) to afford intermediate compound 3

Into a round-bottom flask were added intermediate compound 3, DCM/TFA (3/1) at room temperature. The resulting mixture was stirred for 2 h at room temperature. The resulting mixture was concentrated under reduced pressure. The crude product was purified by Prep-HPLC to afford compound 20. MS (ESI) (m/z) [M+H]+ calculated: 625.3; found: 625.3.

### Example 14: Synthesis of Compound 21

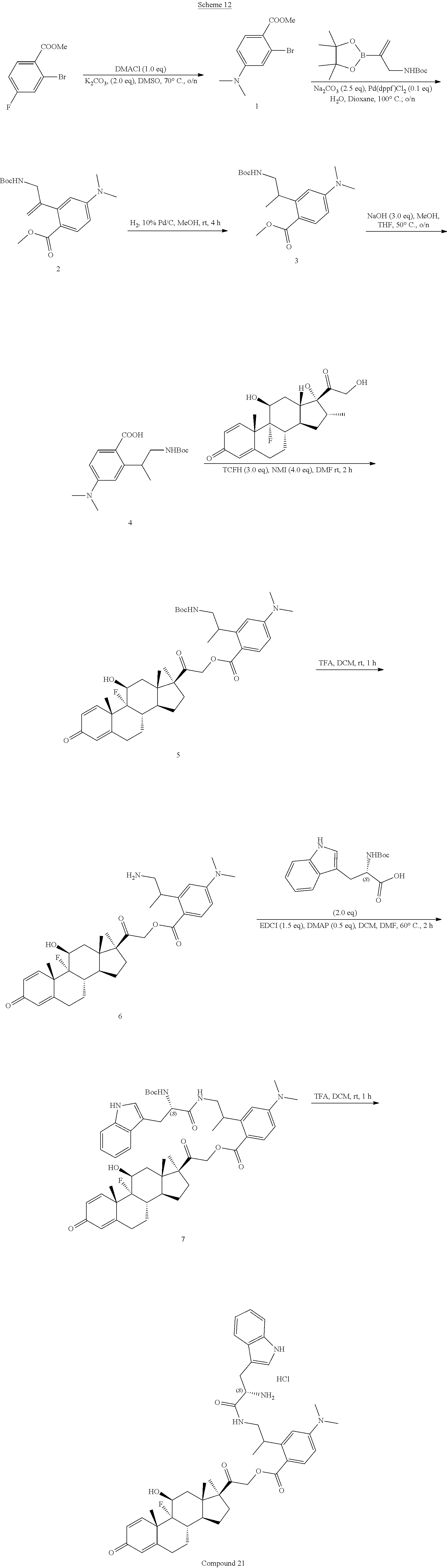

To a stirred solution of methyl 2-bromo-4-fluorobenzoate (2 g, 8.58 mmol, 1 equiv) and Dimethylamine hydrochloride (0.70 g, 8.58 mmol, 1 equiv) in DMSO (8 mL) were added K₂CO₃ (2.97 g, 21.45 mmol, 2.5 equiv) at room temperature. The resulting mixture was stirred overnight at 70° C. The resulting mixture was diluted with EtOAc (30 mL) and water (50 mL). The resulting mixture was extracted with EtOAc (2×50 mL). The combined organic layers were washed with Brine (2×100 mL), dried over anhydrous Na₂SO₄. The residue was purified by silica gel column chromatography, eluted with PE/EA (1:1) to afford intermediate compound 1 (2.1 g, 94.80%) as a yellow oil.

To a stirred solution of intermediate compound 1 (1.9 g, 7.36 mmol, 1 equiv) and tert-butyl N-[2-(4,4,5,5-tetramethyl-1,3,2-dioxaborolan-2-yl) prop-2-en-1-yl]carbamate (4.17 g, 14.72 mmol, 2.00 equiv) in dioxane (10 mL) and H₂O (1 mL) were added Na2CO3 (1.95 g, 18.42 mmol, 2.5 equiv) and Pd(dppf)Cl₂ (0.54 g, 0.73 mmol, 0.1 equiv) at room temperature. The resulting mixture was stirred overnight at 100° C. The resulting mixture was diluted with EtOAc (30 mL) and water (30 mL). The resulting mixture was extracted with EtOAc (2×50 mL). The combined organic layers were washed with Brine (2×100 mL), dried over anhydrous Na₂SO₄. The residue was purified by silica gel column chromatography, eluted with PE/EA (3:1) to afford intermediate compound 2 (944 mg, 38.35%) as a yellow oil.

To a solution of intermediate compound 2 (920 mg, 2.75 mmol, 1 equiv) in MeOH (9 mL) were added 10% Pd/C (460 mg) at room temperature. The resulting mixture was stirred for 4 h at room temperature under hydrogen atmosphere. The resulting mixture was diluted with MeOH (60 mL). The resulting mixture was filtered through a Celite pad and concentrated under reduced pressure. This resulted in intermediate compound 3 (840 mg, 90.76%) as a light yellow solid.

To a stirred solution of intermediate compound 3 (0.38 g, 1.13 mmol, 1 equiv) in THF (3 mL) and MEOH (3 mL) was added 3N NaOH (1.13 mL, 3.39 mmol, 3 equiv) at room temperature. The resulting mixture was stirred overnight at 50° C. The mixture was acidified to pH 5 with 1N HCl. The resulting mixture was extracted with EtOAc (2×50 mL). The combined organic layers were washed with water (2×100 mL), dried over anhydrous Na₂SO₄. After filtration, the filtrate was concentrated under reduced pressure. This resulted in intermediate compound 4 (280 mg, 76.89%) as a light yellow solid.

To a stirred solution of intermediate compound 4 (230 mg, 0.71 mmol, 1 equiv) and dexamethasone (279.98 mg, 0.71 mmol, 1 equiv) in DMF (5 mL) were added NMI (234.29 mg, 2.85 mmol, 4 equiv) and TCFH (600.49 mg, 2.13 mmol, 3 equiv) at room temperature. The resulting mixture was stirred for 2 h at room temperature. The resulting mixture was diluted with EtOAc (10 mL) and water (10 mL). The resulting mixture was extracted with EtOAc (2×30 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EA (1:2) to afford intermediate compound 5 (180 mg, 36.21%) as a white solid.

Into a 8 mL vial were added intermediate compound 5 (120 mg, 0.17 mmol, 1 equiv), DCM (1.2 mL) and TFA (0.4 mL) at room temperature. The resulting mixture was stirred for 1 h at room temperature. The mixture was basified to pH 8 with saturated NaHCO₃ (aq.). The resulting mixture was extracted with EtOAc (3×30 mL). The combined organic layers were washed with brine (3×30 mL), dried over anhydrous Na₂SO₄. After filtration, the filtrate was concentrated under reduced pressure. This resulted in intermediate compound 6 (100 mg, 97.31%) as a white solid. The crude product was used in the next step directly without further purification.

To a stirred solution of intermediate compound 6 (1 equiv) and amino acid (2 equiv) in DMF were added HATU (1.5 equiv) and DIEA (5 equiv) at room temperature. The resulting mixture was stirred for 2 h at 60° C. The resulting mixture was diluted with EtOAc and water. The resulting mixture was extracted with EtOAc. The combined organic layers were washed with brine, dried over anhydrous Na₂SO₄. After filtration, the filtrate was concentrated under reduced pressure. The residue was purified by Prep-TLC (PE/EA 1:1) to afford intermediate compound 7.

Into a round-bottom flask were added intermediate compound 7, DCM/TFA (3/1) at room temperature. The resulting mixture was stirred for 1 h at room temperature. The resulting mixture was concentrated under reduced pressure. The crude product was purified by Prep-HPLC to afford compound 21. MS (ESI)(m/z) [M+H]⁺ calculated: 782.4; found: 782.4.

### Example 15: Synthesis of Compound 22

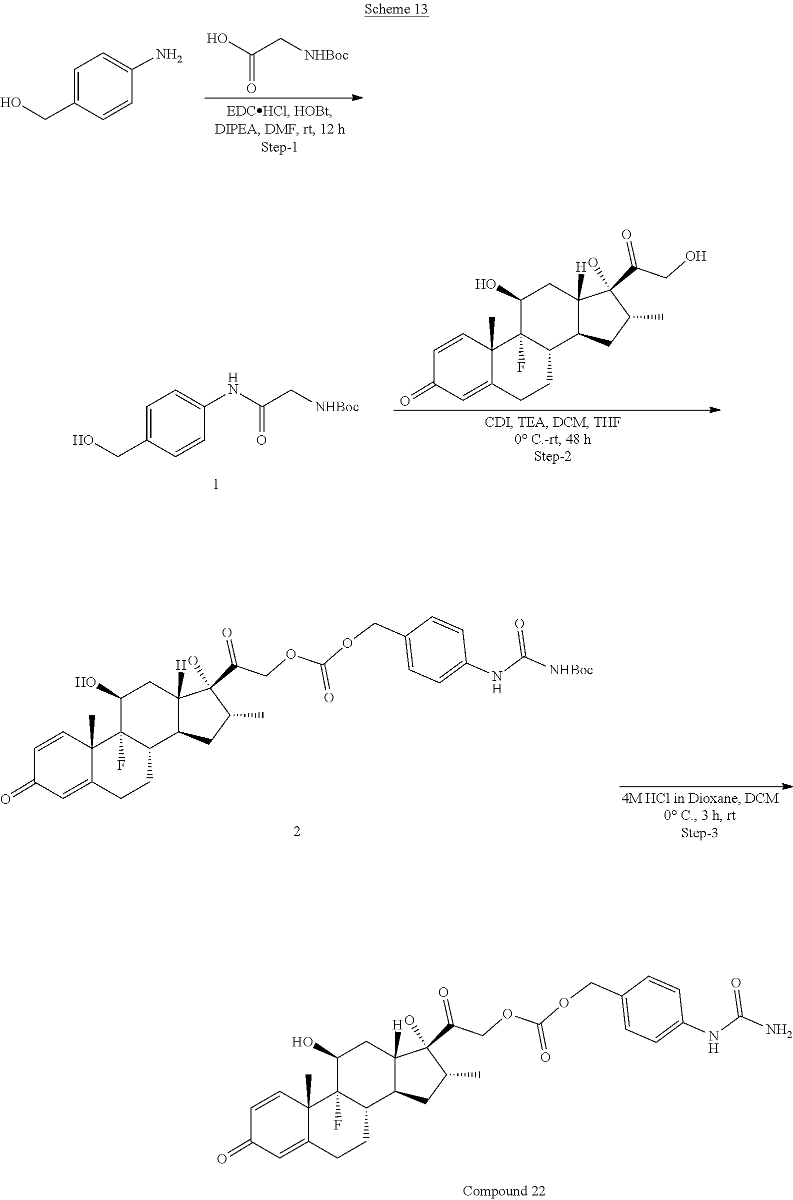

To a stirred solution of (tert-butoxycarbonyl) glycine (3.2 g, 17.864 mmol) in DMF (15 mL) was added HOBt (3.3 g, 24.360 mmol) and DIPEA (8.5 ml, 48.721 mmol). Then (4-aminophenyl) methanol (2 g, 16.240 mmol) and EDC·HCl (4.7 g, 24.360 mmol) were added at 0° C. and the resulting mixture was stirred for 12 h at room temperature and monitored by TLC. The reaction mixture was quenched with ice water (200 mL) and extracted with ethyl acetate (2×300 mL). The combined organic extract was washed with brine solution, dried over sodium sulphate and concentrated under reduced pressure to get the crude. The crude was purified by flash chromatography and the product was eluted in 50 to 60% of EtOAc in pet ether gradient to afford intermediate compound 1 (1.6 g, 70.4%) as a yellow oil.

To a stirred solution of intermediate compound 1 (1 g, 3.571 mmol) in THF and DCM (5:5 mL) was added CDI (2.1 g, 3.571 mmol) at 0° C. and stirred for 20 min. A solution of Dexamethasone (1.4 g, 3.571 mmol) and TEA (1.5 ml, 10.714 mmol) in THF and DCM (5:5 mL) was added and the resulting reaction mixture was stirred for 24 h at ambient temperature. After completion of the reaction the reaction mixture was concentrated under reduced pressure to get crude. The crude was purified by flash chromatography and the product was eluted in 60 to 70% of EtOAc in pet ether gradient to afford intermediate compound 2 (1.55 g, 62.22%) as an off-white solid.

To a stirred solution of intermediate compound 2 (400 mg, 0.573 mmol) in DCM (10 mL) was added 4M HCl in dioxane (8 mL) at 0° C. and the resulting mixture was stirred for 3 h at ambient temperature. After completion of the reaction the solvent was concentrated under reduced pressure to get crude. Water was added and the pH was adjusted to 8.0 with saturated NaHCO₃ solution and extracted with ethyl acetate (2×100 mL). The combined organic extract was washed with brine solution (20 mL) and concentrated under reduced pressure to afford the crude. The crude was purified by preparative HPLC method and lyophilized. The compound was basified with saturated NaHCO₃ solution to neutralize the formic acid traces which could have carried from preparative HPLC purification, and extracted with ethyl acetate (800 mL), then concentrated under reduced pressure and lyophilized to afford Compound 22. MS (ESI) (m/z): [M+1]+ found: 599

### Example 16: Synthesis of Compounds 23-24

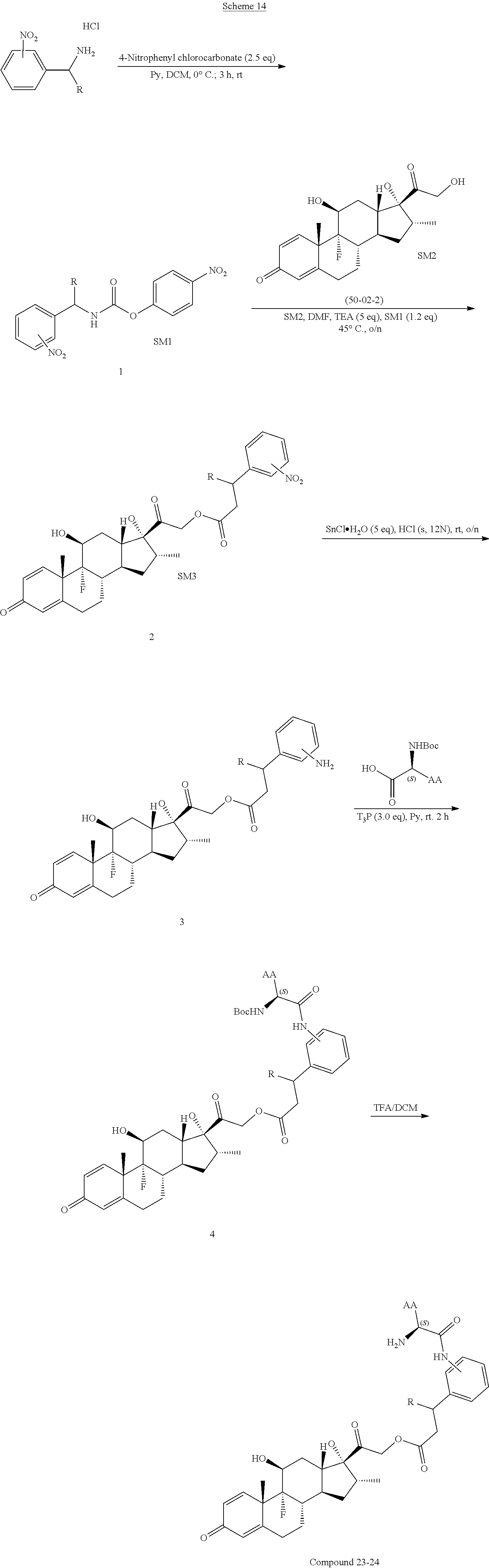

To a stirred solution/mixture of nitrophenyl methanamine analogs (1 equiv) and 4-nitrophenyl carbonochloridate (1.5 equiv) and pyridine (1.5 equiv) in DCM at 0° C. under nitrogen atmosphere. The resulting mixture was stirred for 3 h at room temperature under nitrogen atmosphere. The aqueous layer was extracted with EtOAc (3×30 mL). The resulting mixture was concentrated under reduced pressure. The crude product mixture was used in the next step directly without further purification.

To a stirred solution of nitrophenyl nitrobenzylcarbamate analogs 1 (1.2 equiv) and dexamethasone (3.28 g, 8.369 mmol, 1 equiv) in DMF (10 mL) was added TEA (4.23 g, 41.846 mmol, 5 equiv) at room temperature. The resulting mixture was stirred for 3.0 h at 45° C. under nitrogen atmosphere. Desired product could be detected by LCMS. The resulting mixture was diluted with DCM (40 mL). The resulting mixture was extracted with H₂O (2×2×40 mL). The combined organic layers were washed with brine (2×2×40 mL), dried over anhydrous Na₂SO₄. The residue was purified by silica gel column chromatography, eluted with CH₂Cl₂/EA (5:1) to afford the intermediate compound 2.

Into a round-bottom flask were added intermediate compound 2 (1 equiv), SnCl₂·2H₂O (5 equiv) and HCl (12 N) at room temperature. The resulting mixture was stirred overnight at room temperature. The mixture was basified to pH 8 with saturated NaHCO₃ (aq.). The resulting mixture was extracted with EtOAc. The combined organic layers were washed with brine, dried over anhydrous Na₂SO₄. After filtration, the filtrate was concentrated under reduced pressure. The crude product intermediate compound 3 was used directly in the next step without further purification.

Into a round-bottom flask were added intermediate compound 3 (1 equiv), Py (s), Amino acids (3 equiv) and T3P (3 equiv, 50% in EA) at room temperature. The resulting mixture was stirred for 2 h at room temperature. The resulting mixture was extracted with EtOAc. The combined organic layers were washed with brine, dried over anhydrous Na₂SO₄. After filtration, the filtrate was concentrated under reduced pressure. The residue was purified by silica gel column chromatography, eluted with PE/EA (100%˜70%, 45 min) to afford the product intermediate compound 4.

Into a round-bottom flask were added intermediate compound 4, DCM/TFA (3/1) at room temperature. The resulting mixture was stirred for 2 h at room temperature. The resulting mixture was concentrated under reduced pressure. The crude product was purified by Prep-HPLC to afford the final product Compounds 23-24. Structures of the compounds 23-24 are listed in Table 1. Compound 23: MS (ESI) (m/z) [M+H]⁺ calculated: 654.3; found: 654.4. Compound 24: MS (ESI) (m/z) [M+H]⁺ calculated: 688.3; found: 688.2.

### Example 17: Synthesis of Compounds 25-26

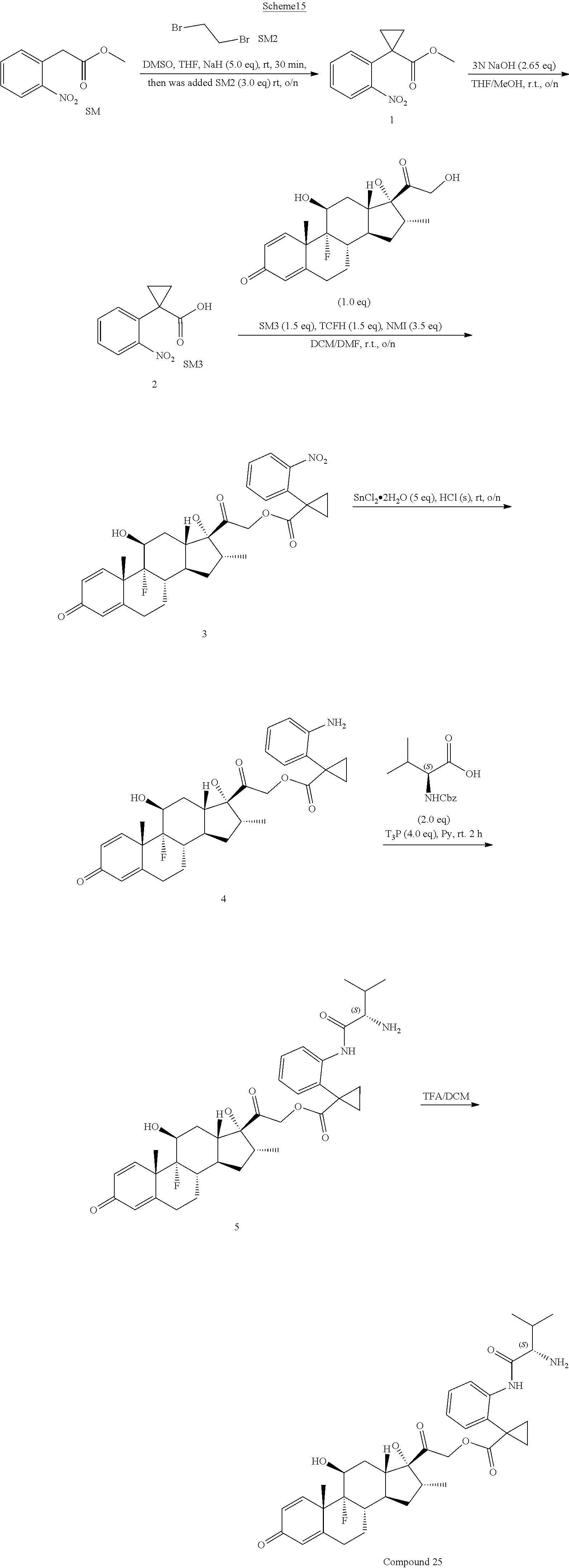

A solution of methyl 2-(2-nitrophenyl)acetate (5 g, 25.6 mmol, 1 equiv) in THF (20 mL) and DMSO (62.5 mL) was prepared at room temperature under nitrogen atmosphere followed by the addition of NaH (5.12 g, 128 mmol, 5 equiv) in portions at 0° C. The resulting mixture was stirred for 30 min at room temperature under nitrogen atmosphere. To the above mixture was added dibromoethane (7 mL, 81.2 mmol, 3.2 equiv) dropwise at room temperature. The resulting mixture was stirred overnight at room temperature. The reaction was quenched with water/ice at 0° C. The aqueous layer was extracted with EtOAc (3×30 mL). The combined organic layers were washed with brine (3×50 mL), dried over anhydrous Na₂SO₄. After filtration, the filtrate was concentrated under reduced pressure. This resulted in intermediate compound 1 (5.5 g, 97.05%) as an orange solid. The crude product was used in the next step directly without further purification. MS (ESI) (m/z): [M+1]=222.

To a stirred solution of intermediate compound 1 (4 g, 18.082 mmol, 1 equiv) in THF (40 mL) and MeOH (40 mL) was added 3 N NaOH (15.97 mL, 47.917 mmol, 2.65 equiv) dropwise at room temperature under nitrogen atmosphere. The resulting mixture was stirred overnight at room temperature under nitrogen atmosphere. The mixture was acidified to pH 6 with 1N HCl. The aqueous layer was extracted with EtOAc (3×50 mL). The combined organic layers were washed with brine (3×50 mL), dried over anhydrous Na₂SO₄. After filtration, the filtrate was concentrated under reduced pressure. The resulting mixture was concentrated under reduced pressure. This resulted in intermediate compound 2 (3.5 g, 93.42%) as a brown solid. The crude product mixture was used in the next step directly without further purification. MS (ESI) (m/z): [M+1]=207.

To a stirred solution of dexamethasone (3.3 g, 8.408 mmol, 1 equiv) and intermediate compound 2 (3.48 g, 16.816 mmol, 2 equiv) in DCM (40 mL) and DMF (40 mL) was added NMI (2.42 g, 29.428 mmol, 3.5 equiv) dropwise at room temperature under nitrogen atmosphere. The resulting mixture was stirred for 30 min at room temperature under nitrogen atmosphere. To the above mixture was added TCFH (3.54 g, 12.612 mmol, 1.5 equiv) dropwise at room temperature. The resulting mixture was stirred overnight at room temperature. The aqueous layer was extracted with EtOAc (3×50 mL). The combined organic layers were washed with brine (3×50 mL), dried over anhydrous Na₂SO₄. After filtration, the filtrate was concentrated under reduced pressure. The residue was purified by silica gel column chromatography, eluted with PE/EA (4:1) to afford intermediate compound 4a (4 g, 81.79%) as a yellow solid. MS (ESI) (m/z): [M+1]=581.

To a stirred mixture of intermediate compound 4a (4 g, 6.877 mmol, 1 equiv) in HCl (12 N, 40 mL) were added SnCl₂·2H₂O (6.95 g, 34.385 mmol, 5 equiv) at room temperature under nitrogen atmosphere. The resulting mixture was stirred overnight at room temperature under nitrogen atmosphere. The mixture was basified to pH 8 with saturated NaHCO₃ (aq.). The resulting mixture was extracted with EtOAc (3×50 mL). The combined organic layers were washed brine (3×50 mL), dried over anhydrous Na₂SO₄. After filtration, the filtrate was concentrated under reduced pressure. The residue was purified by silica gel column chromatography, eluted with PE/EA (100%˜0, 30 min) to afford intermediate compound 4 (3.5 g, 92.26%) as a white solid. MS (ESI) (m/z): [M+1]=551.

To a stirred solution of intermediate compound 4 (300 mg, 0.544 mmol, 1 equiv) and (2S)-2-[(tert-butoxycarbonyl)amino]-3-methylbutanoic acid (354.46 mg, 1.632 mmol, 3 equiv) in pyridine was added T3P (1.38 g, 2.176 mmol, 4 equiv, 50% in EA) at room temperature under nitrogen atmosphere. The resulting mixture was stirred for 2 h at 30° C. under nitrogen atmosphere. The resulting mixture was concentrated under reduced pressure. The residue was purified by silica gel column chromatography, eluted with PE/EA (100%˜50%, 30 min) to afford intermediate compound 5 (200 mg, 48.98%) as a white solid. MS (ESI)(m/z): [M+1]=750.

To a stirred solution of intermediate compound 5 (150 mg, 0.200 mmol, 1 equiv) in DCM) was added TFA (0.5 mL) at room temperature under nitrogen atmosphere. The resulting mixture was stirred for 1 h at room temperature under nitrogen atmosphere. The resulting mixture was concentrated under reduced pressure. The crude product (97.0% was purified by Chiral-Prep-HPLC to afford the final product Compound 25 (58.2 mg, 41.12% yield, HPLC purity:) as a white solid. Compound 26 was prepared in a similar manner by varying the amino acid. Compound 25 MS (ESI) (m/z) [M+H]⁺: 651.3. Compound 26 MS (ESI) (m/z) [M+H]⁺ calculated: 713.4; found: 713.4.

### Example 18: Synthesis of Compounds 27-28

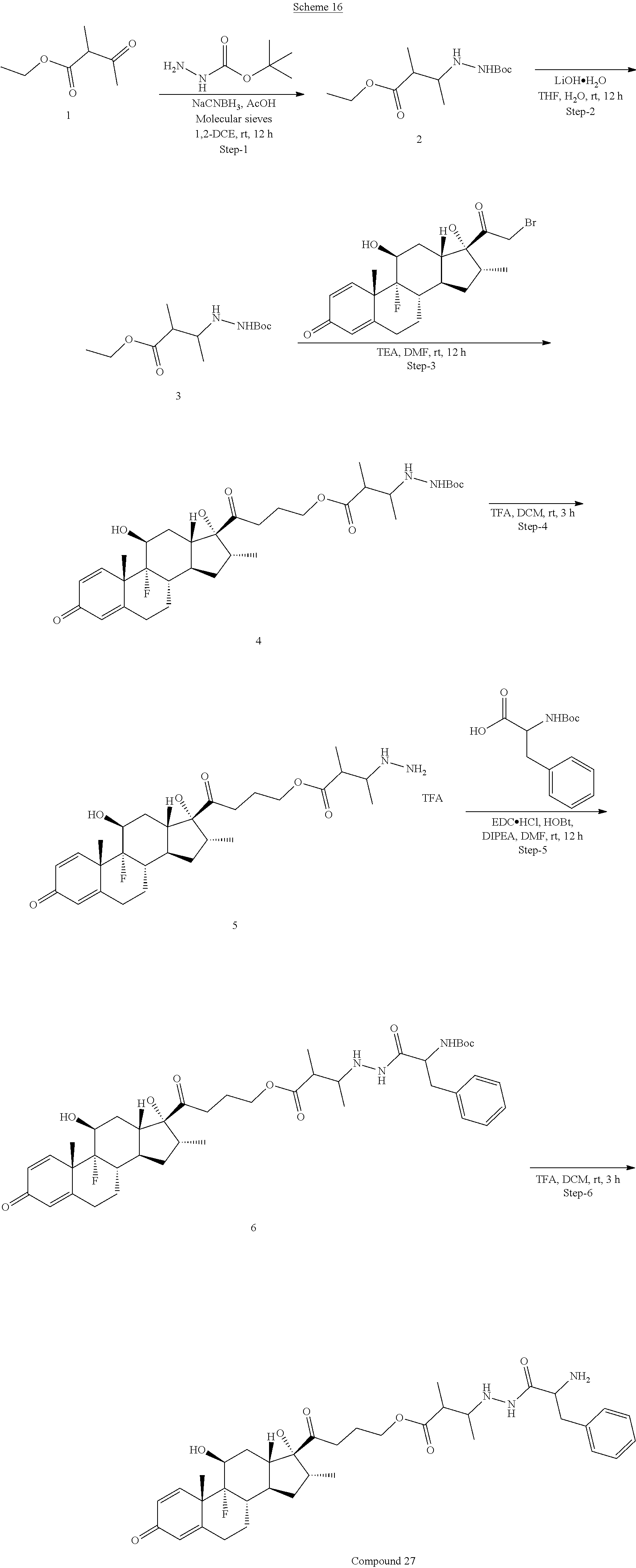

To a stirred solution of Dexamethasone (6 g, 15.30 mmol) in DCM (50 mL) at room temperature were added TPP (4.42 g, 16.83 mmol) and CBr₄ (5.6 g, 16.83 mmol) at 0° C. The resulting mixture was stirred for 3 h at room temperature. After completion of the reaction was quenched with ice water (100 mL) and extracted with DCM (2×300 mL). The combined organic layer washed with brine solution and the organic layer was dried over sodium sulphate, and then concentrated under reduced pressure to get crude. The crude material was purified by flash chromatography and compound eluted with 70 to 80% of ethyl acetate in pet ether to afford brominated dexamethasone (3 g, 43.22%) as pale-yellow solid.

To a stirred solution of ethyl-2-methyl-3-oxobutanoate (10 g, 69.36 mmol) and tert-butyl hydrazine carboxylate (13.75 g, 104.04 mmol) in 1, 2-dichloroethane (100 mL) was added acetic acid (2.07 mL, 34.68 mmol) at 0° C. The resulting mixture was stirred for 2 h at ambient temperature then was added NaCNBH₃ (17.48 g, 277.45 mmol) at 0° C. The reaction mixture was warmed to room temperature and stirred for TLC monitored 12 h. Completion of the reaction was quenched with ice water (200 mL) and extracted with DCM (2×300 mL). The combined organic layer washed with brine solution and the organic layer was dried over sodium sulphate, then concentrated under reduced pressure to get crude. The crude product was purified by flash column chromatography and compound eluted with 10-15% of ethyl acetate in pet ether gradient to afford intermediate compound 1 (12 g, 66.6%) as colorless oil.

To a stirred solution of intermediate compound 1 (5 g, 19.23 mmol) in THF and water (21 mL & 7 mL) was added LiOH·H₂O (5 g, 57.69 mmol) at 0° C. The resulting reaction mixture was stirred at ambient temperature for 12 h. After completion of the reaction, the solvent was distilled off to get the residue. Water was added and the aqueous portion was washed with diethyl ether (30 mL). The layers were separated and the aqueous portion was cooled to 0° C. and acidified to pH 5.0 with 10% KHSO₄ solution. Then extracted with ethyl acetate (2×200 mL) and the combined organic extract dried over Na₂SO₄ and concentrated under reduced pressure to afford the intermediate compound 2 as colorless liquid.

To a stirred solution of brominated dexamethasone (4 g, 6.60 mmol) in DCM (20 mL) was added TFA (5 mL) at 0° C. and resulting reaction mixture was stirred for 3 h at ambient temperature. After completion of reaction concentrated under reduced pressure to get crude material. The crude material was triturated with diethyl ether (2×50 mL), and concentrated under reduced pressure to afford intermediate compound 4a (crude 2.94 g) as an off white solid.

To a stirred solution of intermediate compound 3 (4 g, 17.2 mmol) in DMF were added triethylamine (7.21 mL, 51.7 mmol) and intermediate compound 4a (7.84 g, 17.2 mmol) at 0° C. The resulting reaction mixture was stirred for 12 h at ambient temperature. After completion of reaction, the reaction mixture was quenched with cold water (200 mL) and extracted with ethyl acetate (2×250 mL). The combined organic layer was washed with cold water (3×200 mL) and brine solution. The organic layer was dried with anhydrous sodium sulphate and concentrated under reduced pressure to get crude. The crude material was purified by flash column chromatography and the product was eluted at 40-50% of ethyl acetate in pet ether gradient to afford intermediate compound 55 (4.2 g, 40%) as pale-yellow solid.

To a stirred solution of (tert-butoxycarbonyl)phenylalanine (176 mg, 0.806 mmol) in DMF (6 mL) were added DIPEA (0.42 mL, 2.41 mmol), EDC·HCl (232 mg, 1.20 mmol) and HOBt (164 mg, 1.20 mmol) at 0° C., allowed to room temperature, and stirred for 10 min. Then intermediate compound 4 (500 mg, 0.806 mmol) was added to the reaction mixture at 0° C. The resulting mixture was stirred for 12 h at ambient temperature and completion of reaction was monitored by TLC. The reaction mixture was quenched with ice water (50 mL) and compound extracted with ethyl acetate (2× 100 mL). The combined organic layer washed with cold water (3×50 mL), brine solution and the organic layer was dried over sodium sulphate, then concentrated under reduced pressure to get crude product. The crude product was purified by flash column chromatography and pure compound eluted with 50% of ethyl acetate in pet ether gradient to intermediate compound 6 (410 mg, 65%) as an off white solid.

To a stirred solution of intermediate compound 6 (400 mg, 0.53 mmol) in DCM (5 mL) was added TFA (1 mL) 0° C. and the resulting reaction mixture was stirred for 3 h at ambient temperature. After completion of reaction concentrated under reduced pressure to get crude material. The crude material was basified with saturated NaHCO₃ solution and extracted with ethyl acetate (2× 60 mL). The combined organic layer was washed with brine solution (20 mL), water and concentrated under reduced pressure to get the crude compound. The crude was purified by flash column chromatography and pure compound was eluted with 9-12% of methanol in DCM gradient to afford the Compound 27 (85 mg, 25.13%) as white solid. Compound 28 was also synthesized in a similar manner by using proline as the amino acid in Step 4. Compound 27 LC-MS (ESI): m/z calculated 653, found 654 (M+H)⁻ Compound 28 LC-MS (ESI): m/z calculated 603, found 604 (M+H)⁺.

### Example 19: Synthesis of Compounds 29-30

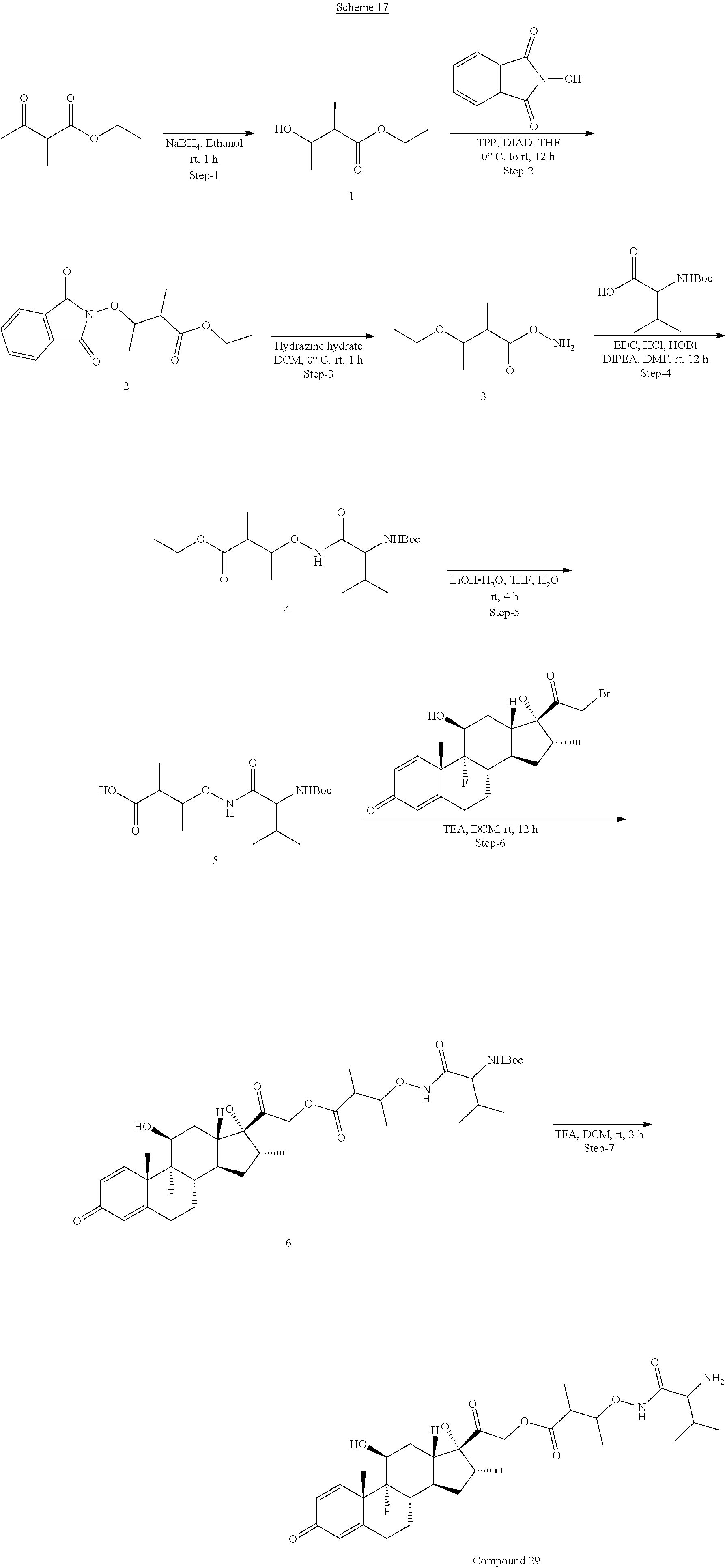

To a stirred solution of ethyl 2-methyl-3-oxobutanoate (10 g, 69.4 mmol) in ethanol (100 mL) was added sodium borohydride (3.94 g, 104 mmol) at 0° C. The resulting mixture was stirred for 1 h at room temperature. The reaction mixture was concentrated under reduced pressure and extracted with ethyl acetate (2×300 mL), washed with water (450 mL) and brine solution (300 mL). The organic layer was dried over sodium sulphate, then concentrated under reduced pressure to get crude intermediate compound 1 (6.1 g) as a colorless liquid.

To a stirred solution of intermediate compound 1 (6 g, 41 mmol) in THF (60 mL) were added triphenylphosphane (16.1 g, 61.6 mmol), DIAD (8.29 mL, 61.6 mmol) and 2-hydroxy-2,3-dihydro-1H-isoindole-1,3-dione (10 g, 61.6 mmol) at 0° C. The resulting mixture was stirred for 16 hours at ambient temperature and monitored by TLC. After completion of the reaction was quenched with water (80 mL) and extracted with ethyl acetate (2×350 ml) and the combined organic layer washed with brine solution and the organic layer was dried over sodium sulphate, then concentrated under reduced pressure to get crude. The crude material was purified by flash chromatography and compound eluted with 20 to 30% of ethyl acetate in hexane to afford the intermediate compound 2 (1.9 g, 15.8%) as brown liquid.

To a stirred solution of intermediate compound 2 (1.9 g, 6.52 mmol) in DCM (20 mL) was added hydrazine hydrate (1.3 g, 26.11 mmol) at 0° C., the resulting mixture was stirred for 1 hour at ambient temperature and reaction was monitored by TLC. The reaction mixture was passed through celite bed and washed with ethyl acetate. The collected filtrate was concentrated under reduced pressure to get crude intermediate compound 3 (927 mg) as a colorless liquid.

To a stirred solution of (tert-butoxycarbonyl) valine (674 mg, 3.1 mmol) in dry DMF (5 mL) were added DIPEA (1.63 mL, 9.31 mmol), EDC·HCl (892 mg, 4.65 mmol) and HOBt (708 mg, 4.65 mmol) at 0° C. and then allowed to rt and stirred for 15 minutes. Intermediate compound 3 (500 mg, 3.1 mmol) was added and the resulting mixture was stirred at ambient temperature for 16 h. The reaction was monitored by TLC and completion of the reaction ice water was added and extracted with ethyl acetate (3×60 mL). The combined organic extract was washed with saturated NaHCO₃ solution (30 mL) and brine solution (30 mL). The organic layer was dried over Na₂SO₄ and concentrated under reduced pressure to get the crude. The crude material was purified by flash chromatography and compound eluted with 20 to 30% of ethyl acetate in pet ether gradient to afford the intermediate compound 4 (490 mg, 43%) as a colorless liquid.

To a stirred solution of intermediate compound 4 (480 mg, 1.36 mmol) in THF (3 mL) and water (1 mL) mixture was added LiOH·H₂O (171 mg, 4.08 mmol) at 0° C. and the resulting mixture was stirred at ambient temperature for 4 h. After completion of the reaction, the solvent was distilled off to get the residue. Water was added and the aqueous portion was washed with diethyl ether (8 mL). The layers were separated and the aqueous portion was cooled to 0° C. and acidified to pH 5.0 with 10% KHSO4 solution and extracted with ethyl acetate (3× 80 mL) and the combined organic extract dried over Na₂SO₄ and concentrated under reduced pressure to intermediate compound 5 (crude 320 mg) as an off-white solid.

To a stirred solution of intermediate compound 5 (300 mg, 0.9 mmol) and dexamethasone (410 mg, 0.9 mmol) in DMF (12 mL) was added TEA (0.379 ml, 2.7 mmol) at 0° C. The resulting mixture was stirred for 16 h at room temperature and monitored by TLC. After completion of the reaction, mixture was quenched with ice water (20 mL) and extracted with ethyl acetate (2×50 mL). The combined organic layer dried over Na₂SO₄ and concentrated under reduced pressure to get the crude. The crude material was purified by flash column chromatography and compound eluted with 40 to 50% of ethyl acetate in pet ether gradient to afford the intermediate compound 56 (210 mg, 31%) as an off white solid.

To a stirred solution of intermediate compound 6 (200 mg, 0.26 mmol) in dry DCM (8 mL) was added Trifluoroacetic acid (2 mL) at 0° C. and the resulting mixture was stirred at ambient temperature for 3 h. After completion of the reaction the solvent was distilled off to get the crude. The crude was dissolved in water (10 mL) cooled to 0° C. and basified to pH 8.0 with saturated NaHCO₃ solution and extracted with 10% MeOH in DCM (2×50 mL) and the combined organic extract was washed with brine solution (10 mL) dried over Na₂SO₄ and concentrated under reduced pressure to get crude. The crude was purified by column chromatography and the product was eluted in 5 to 10% of MeOH in DCM to afford Compound 29 as a white solid. Compound 30 was also synthesized in a similar manner by using phenylalanine as the amino acid in Step 4. Compound 29 LC-MS (ESI): m/=calculated 606, found 607 (M+H)⁺, Compound 30 LC-MS (ESI): m/z calculated 654, found 655 (M+H)+.

### Example 20: Synthesis of Compound 31

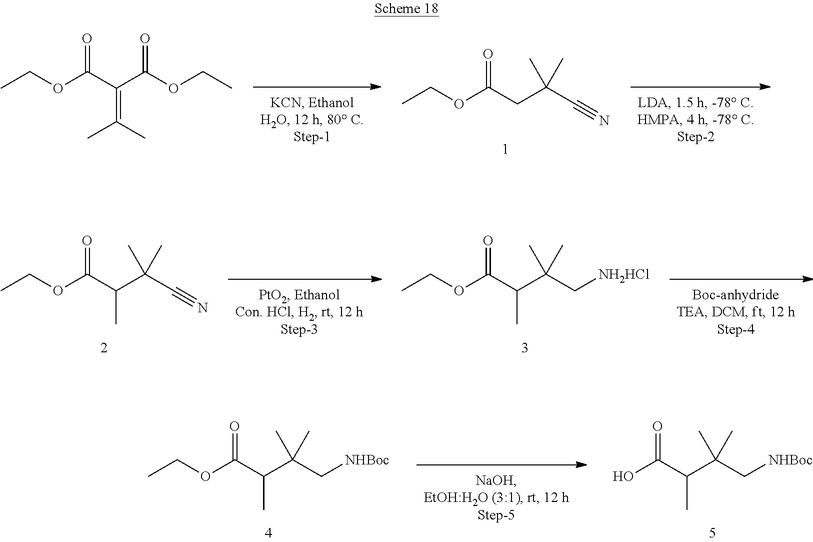

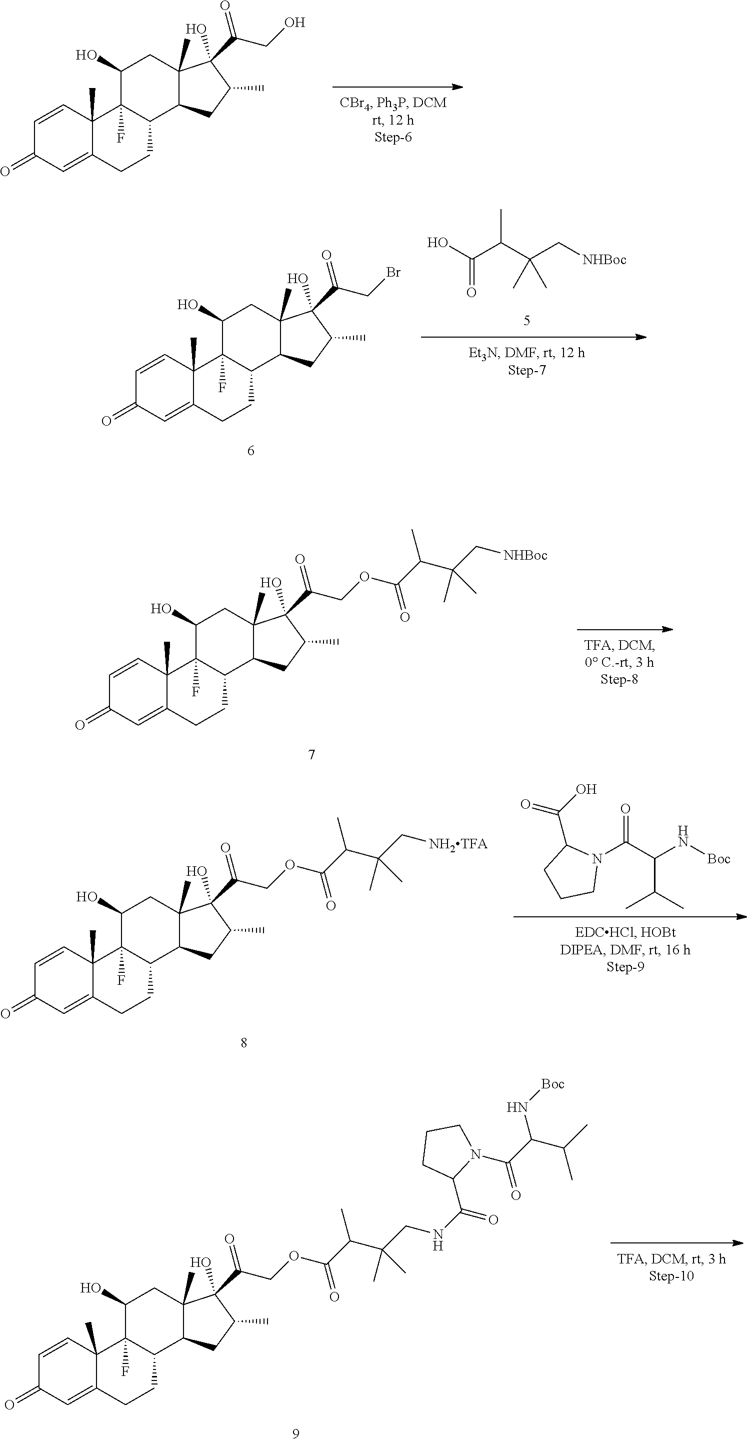

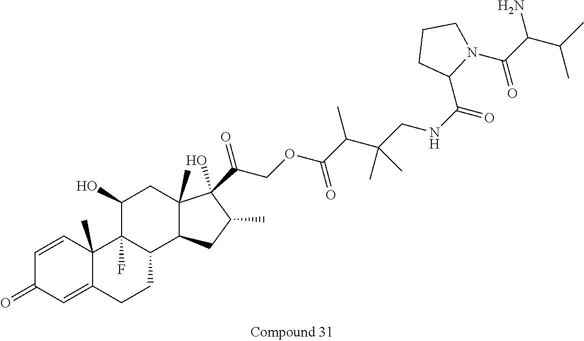

To a stirred solution of diethyl 2-(propan-2-ylidene) malonate (10 g, 49.9 mmol) in ethanol (120 mL) was added a solution of KCN (3.41 g, 54.9 mmol) in water (12 mL) at room temperature and the resulting mixture was heated for 12 h at 80° C. The RM was cooled to RT and the resulting ppt was filtered and washed with ethanol (50 mL). The combined filtrate was acidified to pH 2.0 with 1NHCI (30 mL). The solution was concentrated to get a residue. Water was added and extracted with DCM (100 mL×3). The combined organic layer was washed with 10% aq. Na₂CO₃ solution, dried over Na₂SO₄ and concentrated under reduced pressure to get ethyl 3-cyano-3-methylbutanoate (intermediate compound 1) (crude 5.2 g) as a brown liquid. Thus, obtained crude carried to next step as such without further purification.

To a stirred solution of ethyl 3-cyano-3-methylbutanoate (intermediate compound 1) (2.5 g, 16.12 mmol) in dry THF (60 mL) was added LDA (2M in THF) (12 mL, 24.1 mmol) at −78° C. and the mixture was stirred for 1.5 h. Then a solution of Methyl iodide (3 mL, 48.3 mmol) and HMPA (5.7 g, 32.2 mmol) in THF (10 mL) was added slowly to the reaction mixture and the resulting mixture was stirred for 4 h at −78° C. Completion of the reaction was monitored by TLC. The reaction mixture was quenched with saturated ammonium chloride solution (100 mL) and water (20 mL) and extracted with diethyl ether (100 mL×3). The combined organic layer was washed with brine solution and dried over Na₂SO₄, then concentrated under reduced pressure to get the crude. Thus obtained crude was purified by flash chromatography and the product was eluted in 6%-8% of EtOAc in pet ether to afford ethyl 3-cyano-2,3-dimethylbutanoate (intermediate compound 2) (1.4 g, 51.8%) as a brown liquid.

To a stirred solution of ethyl 3-cyano-2,3-dimethylbutanoate (intermediate compound 2) (1 g, 4.78 mmol) in ethanol (15 mL) was added platinum oxide (IV) (0.54 g, 2.39 mmol) and concentrated HCl (1 mL) at room temperature and the resulting mixture was stirred under H₂ atmosphere for 12 hours. Completion of the reaction was monitored by TLC. The reaction mixture was filtered through celite and the filtrate was concentrated under reduced pressure to get crude. Thus obtained crude was washed with diethyl ether and dried under vacuum to afford ethyl 4-amino-2,3,3-trimethylbutanoate hydrochloride (intermediate compound 3) (crude 780 mg) as an off white solid.

To a stirred solution of ethyl 4-amino-2,3,3-trimethylbutanoate hydrochloride (intermediate compound 3) (2.9 g, 13.8 mmol) in DCM (29 mL) was added triethylamine (9.6 mL, 69 mmol) at 0° C. and stirred for 10 mins. Then Boc-anhydride (4.77 mL, 20.8 mmol) was added and the resulting mixture was stirred at room temperature for 12 hours. After completion of the reaction the reaction mixture was quenched water (50 mL) and extracted with DCM (100 mL×3). The combined organic layer was washed with brine solution and dried over Na₂SO₄ and concentrated under reduced pressure to get the crude. Thus obtained crude was purified by flash chromatography and the product was eluted in 8% to 10% EtOAc in pet ether to afford ethyl 4-((tert-butoxycarbonyl)amino)-2,3,3-trimethylbutanoate (intermediate compound 4) (2.5 g, 67.5%) as a brown

To a stirred solution of ethyl 4-((tert-butoxycarbonyl)amino)-2,3,3-trimethylbutanoate (intermediate compound 4) (500 mg, 1.83 mmol) in Ethanol (10 mL) was added a solution of NaOH (0.14 g, 3.66 mmol) in H₂O (2 mL) at 0° C. and the resulting mixture was stirred for 12 h at room temperature. Completion of the reaction monitored by TLC. The reaction mixture was concentrated under reduced pressure to get residue. The residue was diluted with water (5 mL) and washed with diethyl ether (20 mL). The aqueous layer was acidified to pH 2-3 with 10% KHSO₄ solution at 0° C. and extracted with ethyl acetate (50 mL×3). The combined organic layer was dried over Na₂SO₄ and concentrated under reduced pressure to get 4-((tert-butoxycarbonyl)amino)-2,3,3-trimethylbutanoic acid (intermediate compound 5) (crude 240 mg) as colorless gummy solid.

To a stirred solution of Dexamethasone (6 g, 15.30 mmol) in DCM (50 mL) at room temperature were added TPP (4.42 g, 16.83 mmol) and CBr₄ (5.6 g, 16.83 mmol) at 0° C. The resulting mixture was stirred for 3 h at room temperature. After completion of the reaction, quenched with ice cold water (100 mL) and extracted with DCM (2×300 mL). The combined organic layer was washed with brine solution and the organic layer was dried over sodium sulphate, then concentrated under reduced pressure to get crude. The crude material was purified by flash chromatography and compound eluted with 70% to 80% ethyl acetate in pet ether to afford the intermediate compound 6 (3 g, 43.22%) as pale-yellow solid.

To a stirred solution of (tert-butoxycarbonyl) valylproline (intermediate compound 5) (0.153 g, 0.486 mmol) in dry DMF (5 mL) was added DIPEA (0.3 mL, 1.461 mmol) at 0° C. followed by the addition of EDC·HCl (0.14 g, 0.73 mmol) and HOBt (0.098 g, 0.73 mmol) and stirred at 0° C. for 15 minutes. Intermediate compound 6 (0.3 g, 0.486 mmol) was added and the resulting mixture was stirred at ambient temperature for 16 h. Ice was added and extracted with ethyl acetate (3×50 mL). The combined organic extract was washed with saturated NaHCO₃ solution (20 mL), ice cold water (30 mL) and brine solution (20 mL) dried over Na₂SO₄ and concentrated under reduced pressure to get the crude. The crude was purified by column chromatography and the product was eluted in 70% EA in Hexane to afford intermediate compound 7 as an off white solid (0.26 g, 66.6% yield).

To a stirred solution of intermediate compound 7 (0.12 g, 0.147 mmol) in dry DCM (8 mL) was added Trifluoroacetic acid (2 mL) at 0° C. and the resulting mixture was stirred at ambient temperature for 3 h. After completion of the reaction the solvent was distilled off to get the crude. The crude was dissolved in water (10 mL) cooled to 0° C. and basified to pH 8.0 with saturated NaHCO₃ solution. Extracted with 10% MeOH in DCM (2×50 mL) and the combined organic extract was washed with brine solution (10 mL) dried over Na₂SO₄ and concentrated under reduced pressure to get crude. The crude was purified by column chromatography and the product was eluted in 10% MeOH in DCM to afford Compound 31 as a white solid (0.033 g, 33%). LC-MS (ESI): m/z calculated 715, found 716 (M+H)+.

### Example 21: Synthesis of Compound 32

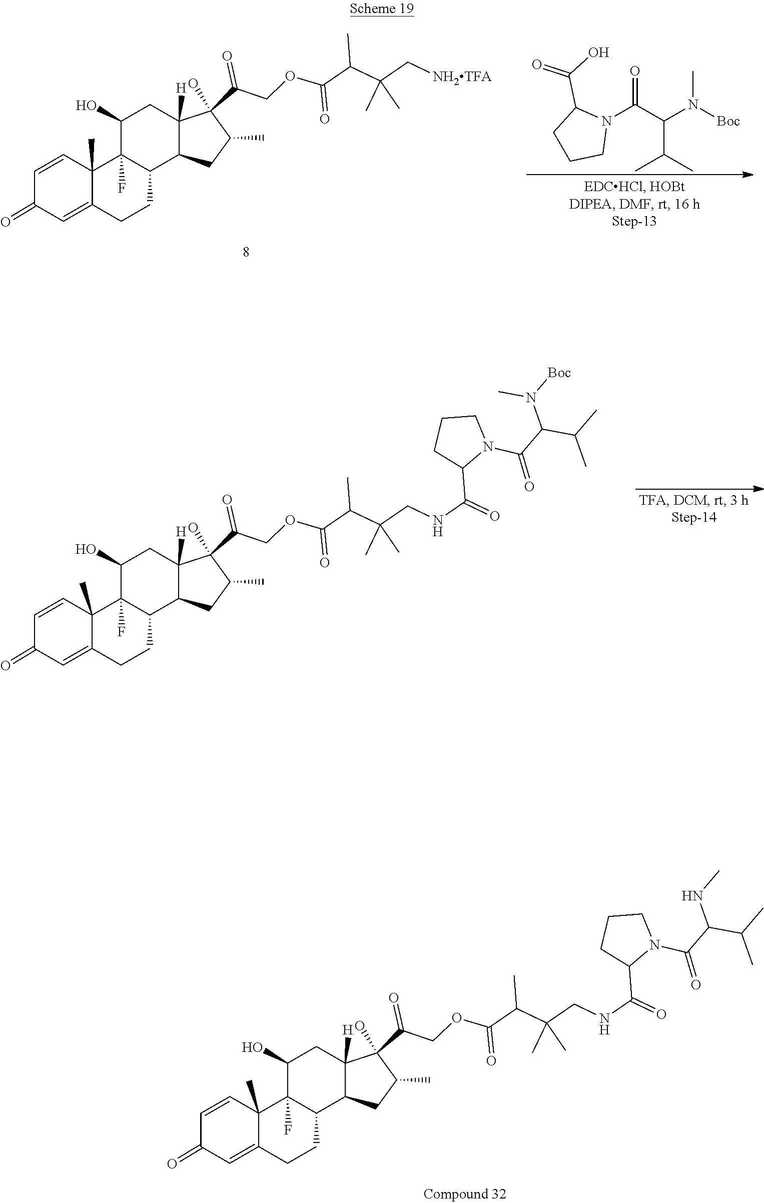

To a stirred solution of N-(tert-butoxycarbonyl)-N-methylvalylproline (190 mg, 0.58 mmol) in dry DMF (5 mL) was added DIPEA (0.3 mL, 1.73 mmol) at 0° C. followed by the addition of EDC·HCl (166 mg, 0.86 mmol) and HOBt (132 mg, 0.86 mmol) and stirred at 0° C. for 15 minutes. Intermediate compound 8 from Example 31 (300 mg, 0.58 mmol) was added and the resulting mixture was stirred at ambient temperature for 16 h. Ice was added and extracted with ethyl acetate (3×50 mL). The combined organic extract was washed with saturated NaHCO₃ solution (20 mL), ice cold water (30 mL) and brine solution (20 mL) dried over Na₂SO₄ and concentrated under reduced pressure to get the crude. The crude was purified by column chromatography and the product was eluted in 70% ethyl acetate in pet ether gradient to afford Compound 32 (0.35 g, 73.04%) as an off white solid. LC-MS (ESI): m/z calculated 829, found 730.

### Example 22: Synthesis of Compound 33

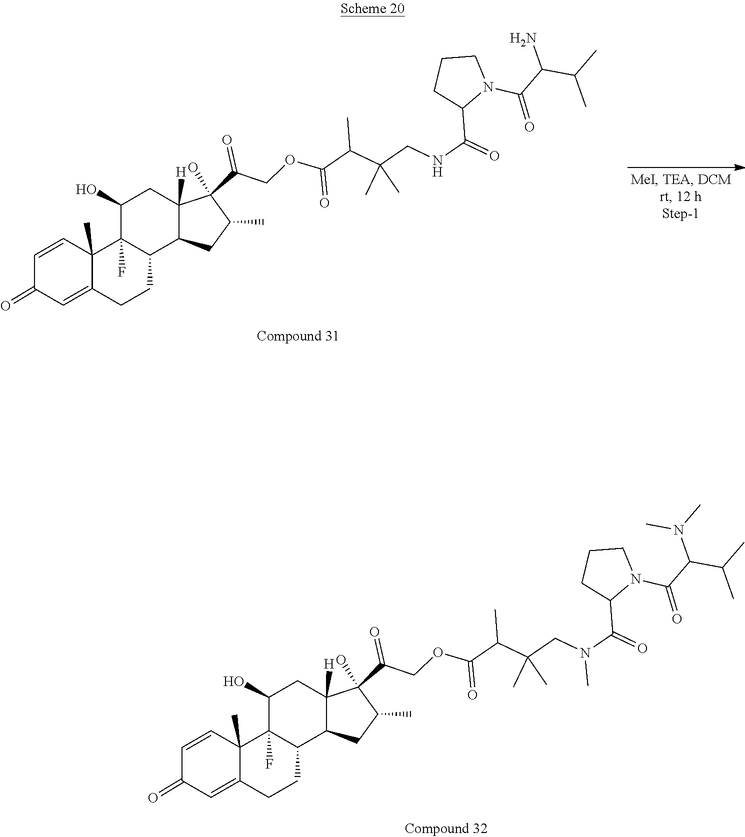

To a stirred solution of Compound 31 (0.65 g, 0.908 mmol) in dry DMF (5 mL) were added potassium carbonate (0.627 g, 4.54 mmol), iodomethane (0.3 mL, 4.54 mmol) at 0° C. and the resulting mixture was stirred for 12 h at room temperature and monitored by TLC. The reaction mixture was quenched with ice water (20 mL) and compound extracted with Ethyl acetate (2×100 mL). The combined organic layer washed with brine solution and the organic layer was dried over sodium sulphate, then concentrated under reduced pressure to get crude. The crude was purified using Prep HPLC method to afford Compound 32 as a white solid (150 mg, 21.8%). LC-MS (ESI): m/z calculated 757, found 758 (M+H)⁺.

### Example 23: Synthesis of Compound 34

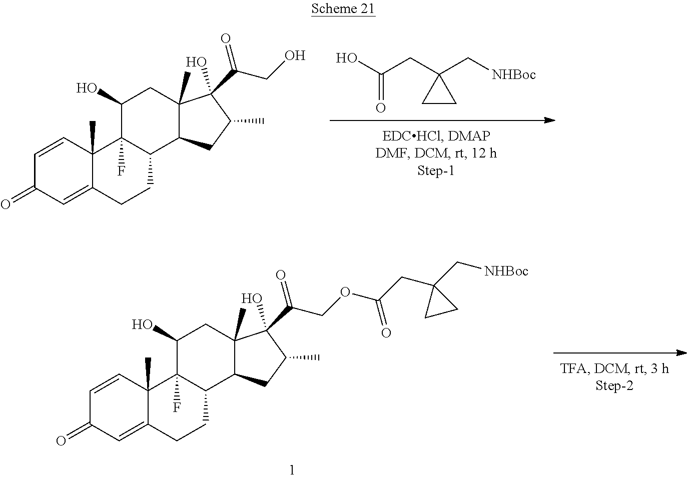

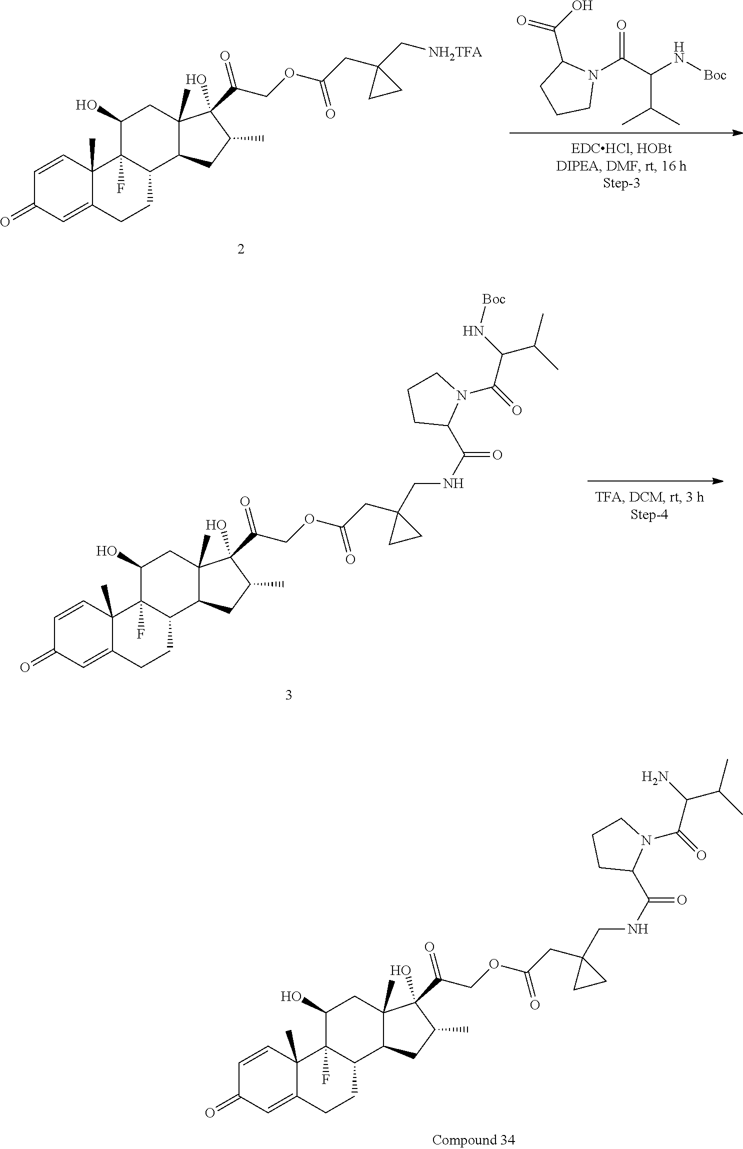

To a stirred solution of 2-(1-(((tert-butoxycarbonyl)amino)methyl)cyclopropyl) acetic acid (2.92 g, 12.75 mmol) in dry DCM (15 mL) and DMF (10 mL) were added Dexamethasone (5 g, 12.73 mmol) and DMAP (460 mg, 3.82 mmol) at ambient temperature. Then EDC·HCl (2.68 g, 14.03 mmol) was added at 0° C. and the resulting mixture was stirred for 12 h at room temperature. The reaction mixture was quenched with ice cold water (100 mL) and extracted with ethyl acetate (2×250 mL). The combined organic extract was washed with water (200 mL) and brine solution (150 mL) and the organic layer was dried over sodium sulphate, concentrated under reduced pressure to get the crude product. The crude was purified by flash column chromatography and the product was eluted with 30-40% ethyl acetate in pet ether gradient to afford intermediate compound 1 (5.5 g, 71%) as an off white solid.

To a stirred solution of intermediate compound 1 (100 mg, 0.19 mmol) in DCM (5 mL) at 0° C. was added slowly TFA (1 mL). The resulting mixture was warmed to room temperature and stirred for 3 h. The reaction was monitored by TLC. After completion of the reaction and the reaction mixture was concentrated under reduced pressure to get crude compound. The crude was basified with saturated NaHCO₃ solution and extracted with ethyl acetate, organic layer was washed with water and brine solution and the organic layer was dried over sodium sulphate, then concentrated under reduced pressure and purified by flash column chromatography, product was eluted with 5-6% of methanol in DCM gradient to afford intermediate compound 2 (47 mg, 56%) as a white solid.

To a stirred solution of (tert-butoxycarbonyl) valylproline (0.101 g, 0.324 mmol) in dry DMF (8 mL) was added DIPEA (0.169 mL, 0.972 mmol) at 0° C. followed by the addition of EDC·HCl (0.0931 g, 0.486 mmol) and HOBt (0.065 g, 0.486 mmol) and stirred at 0° C. for 15 minutes. Intermediate compound 2 (0.2 g, 0.324 mmol) was added and the resulting mixture was stirred at ambient temperature for 16 h. Ice was added and extracted with ethyl acetate (2×50 mL). The combined organic extract was washed with saturated NaHCO₃ solution (20 mL), ice cold water (30 mL) and brine solution (20 mL) dried over Na₂SO₄ and concentrated under reduced pressure to get the crude. The crude was purified by column chromatography and the product was eluted in 4% MeOH in DCM to afford intermediate compound 3 as an off white solid (0.17 g, 65.3% yield).

To a stirred solution of intermediate compound 3 (0.155 g, 0.193 mmol) in dry DCM (8 mL) was added trifluoroacetic acid (1.5 mL) at 0° C. and the resulting mixture was stirred at ambient temperature for 3 h. After completion of the reaction the solvent was distilled off to get the crude. The crude was dissolved in water (10 mL) cooled to 0° C. and basified to pH 8.0 with saturated NaHCO₃ solution. Extracted with ethyl acetate (3×50 mL) and the combined organic extract was washed with brine solution (10 mL) dried over Na₂SO₄ and concentrated under vacuum pressure to get crude which was purified by Preparative HPLC to afford Compound 4 (0.059 g, 43.7%) as a white solid. LC-MS (ESI): m/z calculated 699, found 700 (M+H)+.

### Example 24: Synthesis of Compounds 35-36

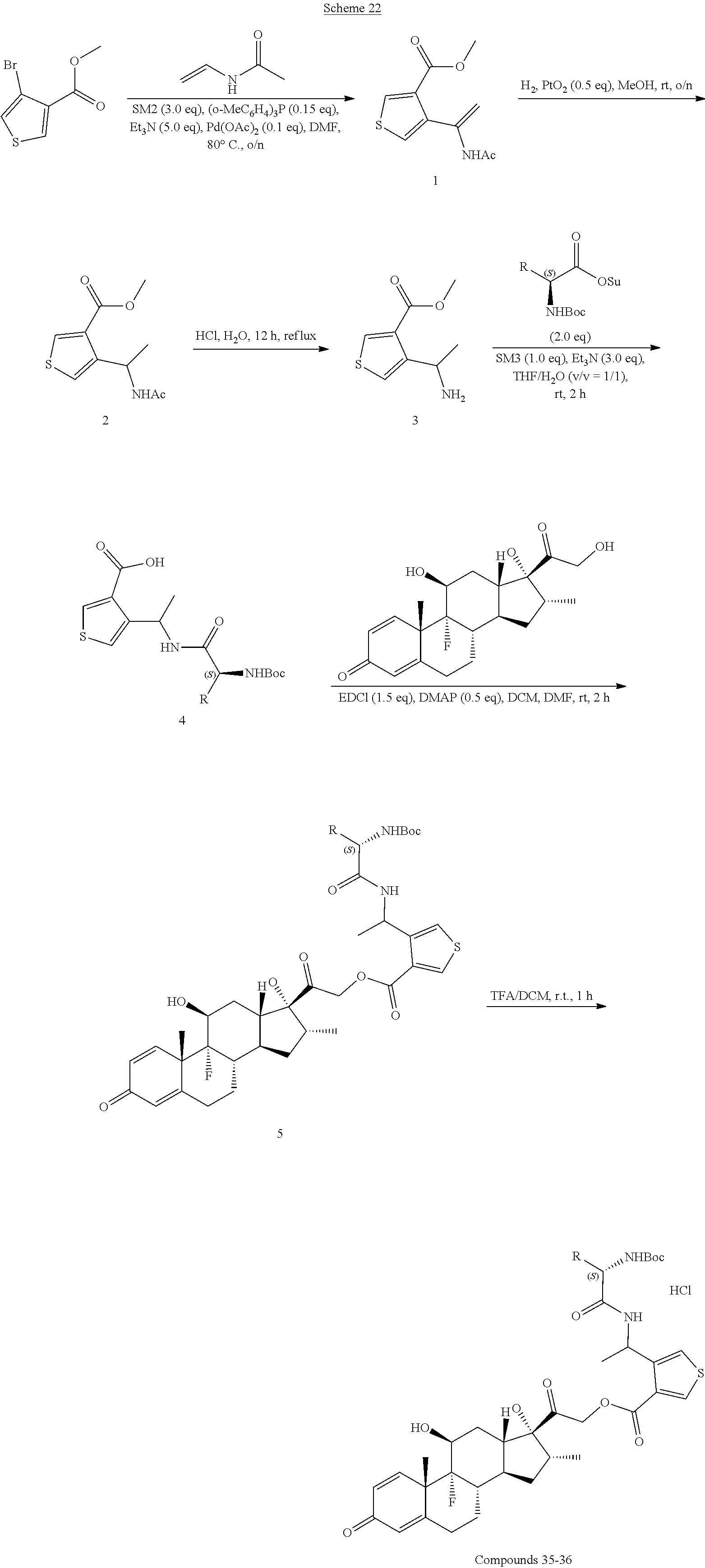

To a stirred solution of methyl 4-bromothiophene-3-carboxylate (4.0 g, 18.09 mmol, 1 equiv) and N-vinylacetamide (4.62 g, 54.28 mmol, 3.0 equiv) in DMF (40 mL) was added tris(2-methylphenyl)phosphane (0.83 g, 2.71 mmol, 0.15 equiv), Pd(AcO)₂ (0.41 g, 1.81 mmol, 0.1 equiv), Et₃N (12.58 mL, 90.47 mmol, 5.0 equiv) at room temperature. The resulting mixture was stirred overnight at 80° C. under nitrogen atmosphere. The resulting mixture was diluted with EtOAc (50 mL) and water (50 mL). The resulting mixture was extracted with EtOAc (3×50 mL). The combined organic layers were washed with water (2×200 mL) and brine (2×300 mL), dried over anhydrous Na₂SO₄. After filtration, the filtrate was concentrated under reduced pressure. The residue was purified by silica gel column chromatography, eluted with PE/EA (1:4) to afford intermediate compound 1 (2.2 g, 53.98%) as a brown yellow oil.

To a stirred mixture of intermediate compound 1 (4.0 g, 17.76 mmol, 1 equiv) and PtO2 (2 g, 8.81 mmol, 0.50 equiv) in MeOH (40 mL) at room temperature. The resulting mixture was stirred overnight at room temperature under hydrogen atmosphere, filtered through a Celite pad and concentrated under reduced pressure. The resulting mixture was concentrated under vacuum. This resulted in intermediate compound 2 (2 g, 46.09%) as a light-yellow solid. To a stirred solution/mixture of intermediate compound 2 (0.8 g, 3.52 mmol, 1 equiv) in H₂O (4 mL) was added HCl (4 mL) at room temperature. The resulting mixture was stirred for 12 h at 100° C. The resulting mixture was concentrated under vacuum. This resulted in intermediate compound 3 (0.6 g, 99.56%) as a yellow solid.

To a stirred solution/mixture of intermediate compound 3 (1 equiv) and Amino acids (2 equiv) in THF and H₂O was added Et₃N (3 equiv) at room temperature. The resulting mixture was stirred for 2.0 h at room temperature. The resulting mixture was diluted with ethyl acetate (20 mL) and water (20 mL). The resulting mixture was extracted with EtOAc (2×40 mL). The combined organic layers were washed with brine (2×40 mL), dried over anhydrous Na₂SO₄. After filtration, the filtrate was concentrated under reduced pressure. The residue was purified by silica gel column chromatography, eluted with PE/EA (1:1) to afford intermediate compound 4

To a stirred solution of intermediate compound 4 (1 equiv) and dexamethasone (1 equiv) in DCM and DMF was added EDCI (1.5 equiv) and DMAP (0.5 equiv) at room temperature. The resulting mixture was stirred for 2.0 h at room temperature. The resulting mixture was diluted with ethyl acetate (20 mL) and water (20 mL). The resulting mixture was extracted with EtOAc (2×40 mL). The combined organic layers were washed with brine (2×100 mL), dried over anhydrous Na₂SO₄. After filtration, the filtrate was concentrated under reduced pressure. The residue was purified by Prep-TLC (PE/EA 1:2) to afford intermediate compound 5.

Into a 20 mL vial were added intermediate compound 5 (1.00 equiv), DCM/TFA (3/1) at room temperature. The resulting mixture was stirred for 1 h at room temperature. The resulting mixture was concentrated under reduced pressure. The crude product was purified by Prep-HPLC to afford compounds 35-36. Compound 35: MS (ESI) (m/z) [M+H]⁺ calculated: 684.3; found: 684.4. Compound 36: MS (ESI) (m/z) [M+H]+ calculated: 693.3; found: 693.3.

### Example 25: Synthesis of Compound 37

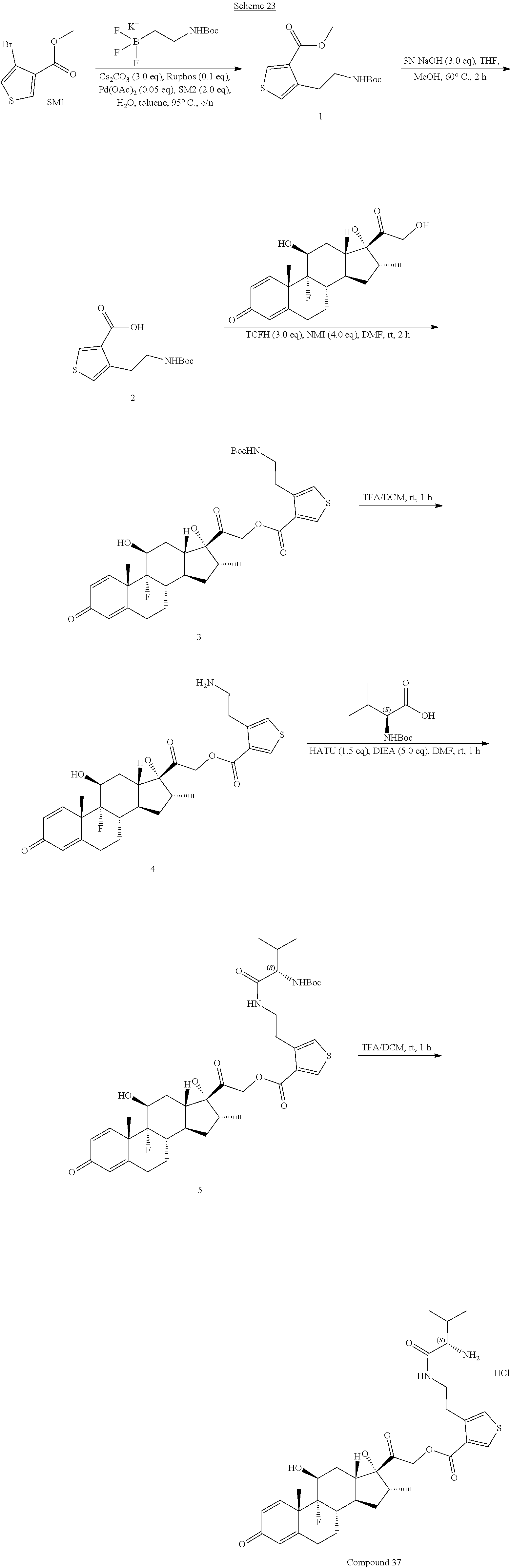

To a solution of methyl 4-bromothiophene-3-carboxylate (1 g, 4.52 mmol, 1 equiv) and potassium tert-butyl N-[2-(trifluoroboranuidyl)ethyl]carbamate (2.27 g, 9.04 mmol, 2 equiv) in toluene (10 mL) and H₂O (1 mL) were added Cs₂CO₃ (1.03 g, 13.57 mmol, 3 equiv), RuPhos (211.09 mg, 0.45 mmol, 0.1 equiv) and Pd(OAc)₂ (50.78 mg, 0.22 mmol, 0.05 equiv). After stirring overnight at 95° C. under a nitrogen atmosphere. The resulting mixture was diluted with EtOAc (20 mL) and water (20 mL). The resulting mixture was extracted with EtOAc (2×40 mL). The combined organic layers were washed with brine (2×40 mL), dried over anhydrous Na₂SO₄. After filtration, the filtrate was concentrated under reduced pressure. The residue was purified by silica gel column chromatography, eluted with PE/EA (1:1) to afford intermediate compound 1 (400 mg, 30.99%) as a white oil.

To a stirred solution of intermediate compound 1 (240 mg, 0.84 mmol, 1 equiv) in THF (2 mL) and MeOH (2 mL) was added 3N NaOH (0.84 mL, 2.52 mmol, 3 equiv) at room temperature. The resulting mixture was stirred for 2 h at 60° C. The mixture was acidified to pH=5 with 1N HCl. The resulting mixture was extracted with EtOAc (2×40 mL). The combined organic layers were washed with brine (2×40 mL), dried over anhydrous Na₂SO₄. After filtration, the filtrate was concentrated under reduced pressure. This resulted in intermediate compound 2 (220 mg, 96.41%) as a white solid.

To a stirred solution/mixture of intermediate compound 2 (0.2 g, 0.73 mmol, 1 equiv) and dexamethasone (0.58 g, 1.47 mmol, 2 equiv) in DMF (2 mL) was added TCFH (0.62 g, 2.21 mmol, 3 equiv) and NMI (0.24 g, 2.92 mmol, 4 equiv) at room temperature. The resulting mixture was stirred for 2 h at room temperature. The resulting mixture was diluted with EtOAc (20 mL) and water (20 mL). The resulting mixture was extracted with EtOAc (2×40 mL). The combined organic layers were washed with brine (2×40 mL), dried over anhydrous Na₂SO₄. The residue was purified by silica gel column chromatography, eluted with PE/EA (1:1) to afford intermediate compound 3 (400 mg, 84.03%) as a white solid.

To a stirred solution of intermediate compound 3 (400 mg, 0.61 mmol, 1 equiv) in DCM (4.5 mL) was added TFA (1.5 mL) at room temperature. The resulting mixture was stirred for 1 h at room temperature. The resulting mixture was diluted with EtOAc (30 mL). The mixture was basified to pH 8 with saturated NaHCO₃ (aq.). The resulting mixture was extracted with EtOAc (2×50 mL). The combined organic layers were washed with brine (1×100 mL), dried over anhydrous Na₂SO₄. After filtration, the filtrate was concentrated under reduced pressure. This resulted in intermediate compound 4 (340 mg, 90.53%) as a light yellow solid.

To a stirred solution of intermediate compound 4 (1 equiv) and amino acid (2 equiv) in DMF were added HATU (1.5 equiv) and DIEA (5 equiv) at room temperature. The resulting mixture was stirred for 1 h at room temperature. The resulting mixture was diluted with EtOAc (10 mL) and water (10 mL). The resulting mixture was extracted with EtOAc (2×30 mL). The combined organic layers were washed with brine (2×50 mL), dried over anhydrous Na₂SO₄. After filtration, the filtrate was concentrated under reduced pressure. The residue was purified by Prep-TLC (PE/EA 1:1) to afford intermediate compound 5.

Into a round-bottom flask were added intermediate compound 5, DCM/TFA (3/1) at room temperature. The resulting mixture was stirred for 1 h at room temperature. The resulting mixture was concentrated under reduced pressure. The crude product was purified by Prep-HPLC to afford compound 37. MS (ESI) (m/z) [M+H]⁺ calculated: 695.3; found: 695.3.

### Example 26: Synthesis of Compound 38

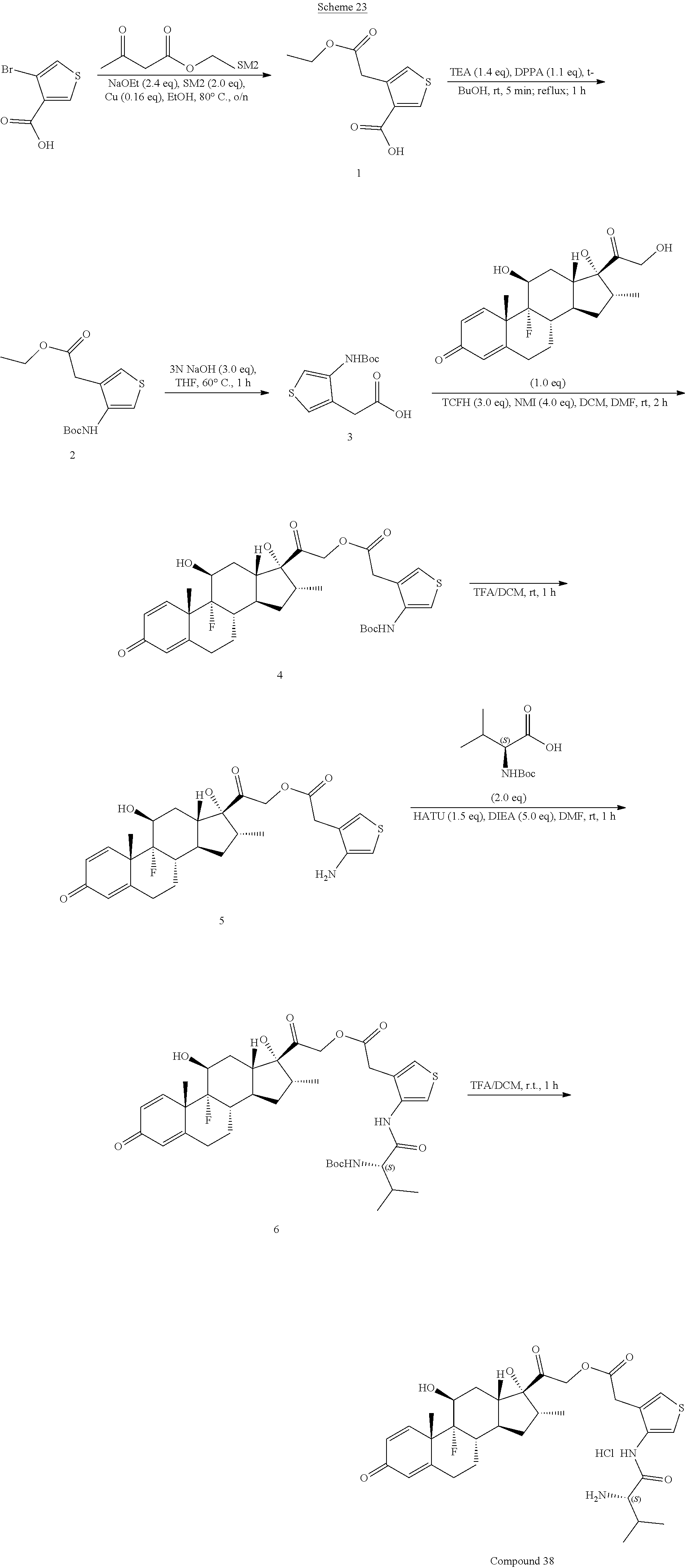

Into a mL 8 vial were added 4-bromothiophene-3-carboxylic acid (1 g, 4.83 mmol, 1 equiv), EtOH (10 mL), ethyl acetoacetate (0.94 g, 7.24 mmol, 1.5 equiv), Cu (49.11 mg, 0.77 mmol, 0.16 equiv) and EtONa (1.31 g, 19.32 mmol, 4 equiv) at room temperature. The resulting mixture was stirred overnight at 80° C. The resulting mixture was diluted with EtOAc (20 mL) and water (20 mL). The mixture was acidified to pH 5 with 1N HCl. The resulting mixture was extracted with EtOAc (2×50 mL). The combined organic layers were washed with brine (2×100 mL), dried over anhydrous Na₂SO₄. After filtration, the filtrate was concentrated under reduced pressure. The residue was purified by Prep-TLC (CH₂Cl₂/MeOH 10:1) to afford intermediate compound 1 (400 mg, 38.66%) as a light yellow solid.

Into a 40 mL vial were added intermediate compound 1 (400 mg, 1.86 mmol, 1 equiv), t-BuOH (4 mL), TEA (0.36 mL, 2.61 mmol, 1.4 equiv) and DPPA (0.44 mL, 2.05 mmol, 1.1 equiv) at room temperature. The resulting mixture was stirred for 1 h at 80° C. The resulting mixture was diluted with water (20 mL). The resulting mixture was extracted with EtOAc (50 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EA (2:1) to afford intermediate compound 2 (130 mg, 24.40%) as a white oil.

Into a 50 mL round-bottom flask were added intermediate compound 2 (130 mg, 0.45 mmol, 1 equiv), THF (2 mL) and NaOH (0.46 mL, 1.36 mmol, 3 equiv) at room temperature. The resulting mixture was stirred for 1 h at 60° C. The mixture was acidified to pH 5 with HCl (aq.). The resulting mixture was extracted with EtOAc (50× mL). The combined organic layers were washed with brine (2×20 mL), dried over anhydrous Na₂SO₄. After filtration, the filtrate was concentrated under reduced pressure. This resulted in intermediate compound 3 (110 mg, 93.84%) as a green oil.

Into a round-bottom flask were added intermediate compound 3 (1 equiv), DMF, dexamethasone (1 equiv), TCFH (3.0 equiv) and NMI (4.0 equiv) at room temperature. The resulting mixture was stirred for 2 h at room temperature. The resulting mixture was extracted with EtOAc. The combined organic layers were washed with brine, dried over anhydrous Na₂SO₄. After filtration, the filtrate was concentrated under reduced pressure. The residue was purified by Prep-TLC (PE/EA 1:2) to afford intermediate compound 4. Into a round-bottom flask were added intermediate compound 4, DCM/TFA (3/1) at room temperature. The resulting mixture was stirred for 1 h at room temperature. The resulting mixture was concentrated under reduced pressure. The crude product intermediate compound 5 was used in the next step directly without further purification.

To a stirred solution of intermediate compound 5 (1 equiv) and amino acid (2 equiv) in DMF were added HATU (1.5 equiv) and DIEA (5 equiv) at room temperature. The resulting mixture was stirred for 1 h at room temperature. The resulting mixture was diluted with EtOAc and water. The resulting mixture was extracted with EtOAc. The combined organic layers were washed with brine, dried over anhydrous Na₂SO₄. After filtration, the filtrate was concentrated under reduced pressure. The residue was purified by Prep-TLC (PE/EA 1:1) to afford intermediate compound 6.

Into a round-bottom flask were added intermediate compound 6, DCM/TFA (3/1) at room temperature. The resulting mixture was stirred for 1 h at room temperature. The resulting mixture was concentrated under reduced pressure. The crude product was purified by Prep-HPLC to afford Compound 38. MS (ESI) (m/z) [M+H]⁺ calculated: 667.2; found: 667.2.

### In-Vitro and In-Vivo Biological Studies

Synthesis of different compounds was followed by testing each compound in different preclinical models for lead optimization. Initial tests included i) chemical stability at two different pH (4.5 and 7.4), ii) human and rodent plasma stability iii) rat liver microsomal stability, iv) cellular permeability v) pharmacokinetic and tissue distribution analysis and vi) biological assays such as DPP4 enzymatic cleavage and glucocorticoid receptor (GR) binding were performed.

In vivo PK studies were conducted in accordance with the guidelines of Institutional Animal Care and Use Committee (IACUC). No abnormal clinical symptoms were observed in rats during the entire experiment.

### Chemical Stability

Chemical stability of the prodrug compounds was tested at pH 4.5 and pH 7.4 (pH adjusted in phosphate buffer saline). Both buffer solutions at two pHs were prepared in house. 2 μL of 500 μM stock solution of the prodrug was added to each vial containing 198 μL PBS at pH 7.4 or pH 4.5 and mixed evenly. The assay was performed in duplicate. The final concentration of the test compound was 5 μM. Samples were incubated at 37° C. at 600 rpm. The initiation of the reaction was staggered so all the time points (0, 30, 60, 120, 180 and 240 minutes) are terminated at the same time with 1000 μL cold quench solution (acetonitrile containing internal standards (IS, 200 nM Labetalol, 100 nM Alprazolam and 2 μM Ketoprofen)). Samples from individual vials were used for the different time points. The samples were vortexed for 2 minutes and centrifuged at 3,220 g for 5 minutes at 4° C. 100 μL of the supernatant was transferred to a new plate. The supernatant was diluted with 100 μL or 200 μL water according to the LC/MS signal response and peak shape. The samples were mixed well and analyzed using LC/MS/MS. The remaining percentage of the parent drug versus reaction time was used to calculate the t1/2 value.

### Plasma Stability

Rat and human plasma were obtained from qualified vendors. 5 μL of 500 μM of the test article solution was added to 495 μL of pre-incubated human or rat plasma to reach a final concentration of 5 μM. The final concentration of organic solvents was not more than 0.5%. The assay was performed in duplicate. The reaction samples were incubated in a 37° C. water bath with shaking at approximately 60 rpm. 50 μL of the reaction samples were collected at 0, 30, 60, 120 and 180 minutes. The reaction was stopped by adding 300 μL of room temperature quench solution (acetonitrile containing internal standards (IS, 200 nM labetalol, 100 nM alprazolam and 2 μM ketoprofen)). The samples were vortexed for 5 minutes followed by centrifugation of the samples at 3,220 g for 30 minutes at room temperature to precipitate the protein. 100 μL of the supernatant was transferred to a new plate and diluted with 100 μL or 200 μL water according to the LC/MS signal response and peak shape. The samples were mixed well and analyzed using LC/MS/MS. The remaining percentage of parent drug versus reaction time was used to calculate the t1/2 value.

### Metabolic Stability:

Rat liver microsomes were obtained from qualified vendors and metabolic stability of the test compound was determined at 1 μM. The compound was incubated at 37° C. with 0.5 mg microsome/mL in the buffer in the presence of 1 mM NADPH. Aliquots of 50 μL were taken from the reaction solution at 0, 15, 30, 45 and 60 minutes. The reaction was stopped by the addition of 4 volumes of cold acetonitrile with internal standards (IS, 200 nM labetalol, 100 nM alprazolam and 2 μM ketoprofen). Precipitated proteins were removed by centrifugation, followed by withdrawing and diluting the supernatant for analysis. Two positive controls and negative controls (without NADPH) were used in the study. All samples were analyzed in duplicates. LC/MS/MS was performed for detection and quantification of the test compounds. Linear regression was determined using the remaining percentage of the parent drug at each incubation time and t1/2 value was calculated.

### Permeability Study Using Caco-2 Cell Monolayers:

The objective of this study was to evaluate the bidirectional permeability and absorption mechanisms of the test compounds. CaCO-2 cells were cultured, and a monolayer was developed in-house in 96 well-plates over 18 days using standard protocol. The monolayer was tested for the integrity of the cell monolayer before using.

Test compounds were added to the transwell insert (apical compartment) to determine the rate of drug transport in the apical to basolateral direction. To determine the rate of drug transport in the basolateral to apical direction, the test compound was added to the receiver plate wells (basolateral compartment). Appropriate amount of HBSS (10 mM HEPES, pH 7.4) was added in both compartments for a final volume of 200 μL. The plates were incubated at 37° C. with shaking at 150 rpm on a rotary shaker for 2 hours. At the end of the transport period, 50 μL from the apical and basolateral wells were collected and 4 volume of cold acetonitrile containing internal standards (IS: 100 nM alprazolam, 200 nM labetalol, and 2 μM ketoprofen) was added to each sample to terminate the reaction. The samples were vortexed and centrifuged at 3,800 g for 20 minutes. An aliquot of 150 μL of the supernatant was used for LC/MS/MS analysis. All samples were analyzed in duplicates. Samples were analyzed through LC/MS/MS for estimating the permeability and efflux of the compounds across Caco-2 cell monolayers.

### Glucocorticoid Receptor Binding Assay

Glucocorticoid receptor (GR) binding of prodrugs was determined using TR-FRET GR competitive binding assay. Three-point (0.1, 1 and 10 μM) binding was evaluated for each prodrug. Dexamethasone was used as the positive control.

### DPP4 Assay:

To assess the hydrolysis of prodrugs by DPP4 enzyme, 10 μM of a test compound was incubated with 20 ng/reaction of DPP4 enzyme for 180 min at 37° C. Aliquots were withdrawn at 0, 60 and 120 minutes from the reaction mixture and quenched with 20-volumes of cold acetonitrile. The concentration of the prodrug and free dexamethasone was analyzed using LC/MS. Half-life of the prodrug and percentage of dexamethasone released was calculated for each compound. Glycine-proline p-nitroanilide was used as a positive control for this assay.

### Pharmacokinetic Studies in Rats: Lung

Six to eight weeks old Sprague-Dawley rats were procured from a qualified provider. Test compounds were introduced in rats by a single intravenous administration. A liquid formulation was made (2 mg/kg dexamethasone or equivalent prodrug) in 20% HP-β-CD and administered via the tail vein to the rats which were subject to overnight fasting as a prerequisite. Blood and lung samples were collected at three timepoints (15, 60 and 360 min) from three rats (3 replicates) after dosing. Blood samples (0.1, 0.2 or 0.5 mL) were collected with a 1 mL syringe containing anticoagulants K2EDTA. Blood samples were centrifuged to obtain plasma for analysis. Additional blood samples were collected at 15, 30, 60, 120, 240 min from the 360 min group. After collection at each time point, lung samples were frozen at −75° C. Prior to analysis, a portion of lung sample was weighed and homogenized with phosphate buffer saline. Concentrations of the prodrug and dexamethasone released from the prodrug, in both plasma and lung tissue, were measured by LC/MS/MS. Data analysis and pharmacokinetic parameters were generated using WinNonlin software.

### Pharmacokinetic Studies in Rats: Brain

Six to eight weeks old Sprague-Dawley rats were procured from a qualified provider. Test compounds were introduced in rats by a single intravenous administration. A liquid formulation was made (2 mg/kg dexamethasone or equivalent prodrug) in 20% HP-β-CD and administered via the tail vein to the rats which were subject to overnight fasting as a prerequisite. Blood and brain samples were collected at three timepoints (15, 60 and 360 min) from three rats (3 replicates) after dosing. Blood samples (0.1, 0.2 or 0.5 mL) were collected with a 1 ml syringe containing anticoagulants K2EDTA. Blood samples were centrifuged to obtain plasma for analysis. Additional blood samples were collected at 15, 30, 60, 120, 240 min from the 360 min group. After collection at each time point, lung samples were frozen at −75° C. Prior to analysis, a portion of brain sample was weighed and homogenized with phosphate buffer saline. Concentrations of the prodrug and dexamethasone released from the prodrug, in both plasma and brain tissue, were measured by LC/MS/MS. Data analysis and pharmacokinetic parameters were generated using WinNonlin software.

Table 2 shows the chemical stability at two different pH (4.5 and 7.4) for prodrug compounds according to embodiments of the present invention versus comparative compounds (dexamethasone conjugated to amino acids without the linkers). As shown in Table 2, the prodrug compounds were stable even at pH 7.4 while the comparative compounds were not stable.

TABLE 2
Chemical stability of compounds vs. comparative compounds

| | Chemical Stability, | Chemical Stability, |
|---|---|---|
| Compound | pH 4.5 | pH 7.4 |
| Comparative Compound 1 | Yes | No |
| Comparative Compound 2 | No | No |
| Comparative Compound 3 | Yes | No |
| Compound 1 | Yes | Yes |
| Compound 2 | Yes | Yes |
| Compound 3 | Yes | Yes |
| Compound 7 | Yes | Yes |
| Compound 9 | Yes | Yes |
| Compound 11 | Yes | Yes |
| Compound 12 | Yes | Yes |
| Compound 13 | Yes | Yes |
| Compound 14 | Yes | Yes |
| Compound 15 | Yes | Yes |
| Compound 16 | Yes | Yes |
| Compound 24 | Yes | Yes |
| Compound 25 | Yes | Yes |
| Compound 28 | Yes | Yes |
| Compound 31 | Yes | Yes |
| Compound 34 | Yes | Yes |
| Compound 35 | Yes | Yes |
| Compound 36 | Yes | Yes |
| Compound 37 | Yes | Yes |
| Compound 38 | Yes | Yes |

Table 3 shows human plasma stability and rat plasma stability for prodrug compounds according to embodiments of the present invention versus comparative compounds. As shown in Table 3, the prodrug compounds showed better human plasma stability as compared to comparative compounds.

TABLE 3
Human plasma stability and rat plasma stability
for prodrug compounds vs. comparative compounds.

| | Human Plasma Stability | Rat Plasma Stability |
|---|---|---|
| | t½ min | t½ min |
| Comparative | 173 | NA |
| Compound 1 | | |
| Comparative | 25 | NA |
| Compound 3 | | |
| Compound 1 | >240 | NA |
| Compound 6 | >240 | >240 |
| Compound 9 | >240 | NA |
| Compound 11 | >240 | >240 |
| Compound 12 | >240 | >240 |
| Compound 13 | >240 | >240 |
| Compound 14 | >240 | >240 |
| Compound 15 | >240 | >240 |
| Compound 16 | >240 | >240 |
| Compound 23 | >240 | >240 |
| Compound 24 | >240 | >240 |
| Compound 25 | >240 | >240 |
| Compound 28 | >240 | 147 |
| Compound 31 | >240 | >240 |
| Compound 33 | >240 | >240 |
| Compound 34 | >240 | >240 |
| Compound 35 | >240 | >240 |
| Compound 36 | >240 | >240 |
| Compound 37 | >240 | >240 |
| Compound 38 | >240 | >240 |

Note:
t½ >240 min was considered to be stable

Table 4 shows glucocorticoid receptor (GR) binding data for prodrug compounds according to embodiments of the present invention versus comparative compounds. A shown in Table 4 the prodrugs of the present invention showed significantly lower GR binding as compared to dexamethasone.

TABLE 4
Glucocorticoid receptor (GR) binding data for prodrugs
compounds vs. Dexamethasone and 0.1 μM.

| Compound | GR Binding Data |
|---|---|
| Dexamethasone | 85% |
| Compound 18 | 46% |
| Compound 25 | 19% |

Table 5 shows DPP4 assay data for prodrug compounds according to embodiments of the present invention. As shown in Table 5, DPP4 was effective in cleaving the prodrugs of the present invention.

TABLE 5
DPP4 assay data for prodrugs compounds

| Compound | DPP4 t 1/2 min |
|---|---|
| Compound 3 | 152 |
| Compound 31 | 167 |
| Compound 32 | 214 |

Table 6 shows in-vitro data for prodrug compounds 9, 25 and 31 according to embodiments of the present invention.

TABLE 6
Data from in vitro studies for Compounds 9, 25 and 31

| Assays | Compound 9 | Compound 25 | Compound 31 |
|---|---|---|---|
| Chemical | Yes | Yes | Yes |
| stability | | | |
| Rat plasma | >240 | >240 | >240 |
| stability | | | |
| RLM | 24 | 38 | 63 |
| Permeability | A-B = 0.025 × | A-B = 0.065 × | A-B = 0.065 × |
| | 10⁻⁶, cm/s | 10⁻⁶, cm/s | 10⁻⁶, cm/s |
| | B-A = 7 × | B-A = 11 × | B-A = 11 × |
| | 10⁻⁶, cm/s | 10⁻⁶, cm/s | 10⁻⁶, cm/s |
| | Efflux ratio: 279 | Efflux ratio: 170 | Efflux ratio: 170 |

Biochemical data showed prodrug compounds of the present invention also released active molecules (dexamethasone) in time and dose dependent manner.

### In Vivo Pharmacokinetics and Lung Tissue Distribution

Compounds 9, 25 and 31 were tested in rats and the concentration of the prodrugs and active molecules (dexamethasone) were measured in the plasma and lungs at three different time points. Table 7 shows that the prodrug compounds exhibited higher concentration of dexamethasone in the lung relative to that of in the plasma.

TABLE 7
In-vivo analysis of Dexamethasone, Compound 9, Compound 25, Compound
31 and Dexamethasone released from the prodrugs in rat

| | Time in | Dexa- | Compound | Compound | Compound |
|---|---|---|---|---|---|
| | min | methasone | 9 | 25 | 31 |
| Conc of | 15 | NA | 694 ± 83 | 742 ± 96 | 1427 ± 102 |
| Prodrug in | 60 | NA | 244 ± 13 | 367 ± 55 | 358 ± 16 |
| plasma | 360 | NA | 21 ± 1.5 | 34 ± 10 | 58 ± 3 |
| Conc in | 15 | NA | 992 ± 422 | 3895 ± 368 | 770 ± 122 |
| Prodrug in | 60 | NA | 959 ± 283 | 5827 ± 1483 | 1109 ± 265 |
| lung | 360 | NA | 433 ± 212 | 1425 ± 509 | 792 ± 192 |
| Lung/Plasma | 15 | NA | 148 | 531 | 54 |
| % of Prodrug | 60 | NA | 397 | 1650 | 311 |
| | 360 | NA | 2100 | 4690 | 1370 |
| Conc of Dex | 15 | 1400 ± 731 | 191 ± 15 | 55 ± 8 | 58 ± 3 |
| in plasma | 60 | 1247 ± 28 | 279 ± 27 | 49 ± 7 | 51 ± 2 |
| | 360 | 250 ± 19 | 137 ± 29 | 48 ± 17 | 42 ± 1.5 |
| Conc of Dex | 15 | 620 ± 315 | 617 ± 315 | 348 ± 49 | 43 ± 3 |
| in lung | 60 | 463 ± 139 | 608 ± 196 | 425 ± 158 | 51 ± 6 |
| | 360 | 89 ± 18 | 190 ± 80 | 138 ± 69 | 33 ± 2 |
| Lung/Plasma | 15 | 44 | 320 | 643 | 74 |
| % of Dex | 60 | 37 | 222 | 910 | 100 |
| | 360 | 36 | 136 | 343 | 78 |

The concentration of dexamethasone in plasma released from the prodrugs was significantly lower than free dexamethasone administered to the rats, while the concentration of dexamethasone in the lung was comparable in both cases for up to 60 min. The concentration of dexamethasone released from the prodrugs in the lungs at 6 h was approximately two times higher relative to dexamethasone alone. Therefore, the dexamethasone percentage ratio in lung/plasma was found to be higher by 2-3-fold in Compound 9 and 7-8 fold higher in Compound 25 when compared to dexamethasone alone. Higher abundance of dexamethasone found in the lung was attributed to the fact that the Compounds 9 and 25 released dexamethasone in the lung while maintaining relatively lower concentration in the plasma and other significantly vital organs like brain.

From the in vivo study of dexamethasone, plasma concentration of dexamethasone was 1250-1400 ng/ml for up to 1 h, which can lead to high systemic exposure. The concentration of dexamethasone in the lung was found to be in the range of 450-620 ng/mL (Table 7). At the same time point, Compound 9 released ˜200 ng/ml of dexamethasone in the plasma, however, the concentration in the lung was 608-620 ng/ml (Table 7). Hence, it is possible to achieve efficacious dose in the lung while maintaining a 6-fold lower concentration of dexamethasone in the plasma.

Similarly at the same time point, Compound 25 released ˜50 ng/mL of dexamethasone in the plasma, while the concentration of dexamethasone in the lung was 350-425 ng/mL (Table 7). Hence, it is possible to achieve efficacious dose in the lung while the concentration of dexamethasone in the plasma is 25-30-fold lower.

Thus, the data suggests that prodrugs compounds of the present invention have the potential to deliver adequate amounts of dexamethasone in the lung with less systemic exposure, thereby, reducing the toxicities of dexamethasone in the body.

### In Vivo Pharmacokinetics and Brain Tissue Distribution

Compound 33 was tested in rats and the concentration of the prodrugs and active molecules (dexamethasone) were measured in the plasma and brain at different time points. Table 8 shows that the prodrug compounds exhibited higher concentration of dexamethasone in the brain relative to that of in the plasma.

TABLE 8

| | Time in min | Compound 33 | Dexamethasone |
|---|---|---|---|
| Conc of Prodrug in | 15 | 6549 ± 1537 | NA |
| plasma | 60 | 1349 ± 101 | |
| | 360 | 11 ± 4.17 | |
| | 540 | 5.73 ± 1.02 | |
| | 1440 | 1.70 ± 0.06 | |
| Conc of Prodrug in Brain | 15 | 36.9 ± 15.7 | |
| | 60 | 25.3 ± 8.35 | |
| | 360 | 6.34 ± 4.43 | |
| | 540 | 5.60 ± 1.64 | |
| | 1440 | 5.08 ± 0.74 | |
| Brain/Plasma % of | 15 | 1 | |
| Prodrug | 60 | 2 | |
| | 360 | 59 | |
| | 540 | 98 | |
| | 1440 | 299 | |
| Conc of Dex in plasma | 15 | 18.8 ± 7.75 | 1400 ± 731 |
| | 60 | 8.01 | 1247 ± 27.7 |
| | 360 | 2.74 | 250 ± 18.9 |
| | 540 | 2.09 | NA |
| | 1440 | NA | NA |
| Conc of Dex in Brain | 15 | 16.5 | 54.1 ± 17.2 |
| | 60 | NA | 84.1 ± 7.44 |
| | 360 | 17.4 ± 5.91 | 42.9 ± 5.34 |
| | 540 | 23.3 | NA |
| | 1440 | 24.0 ± 2.0 | NA |
| Brain/Plasma % of Dex | 15 | 88 | 0.04 |
| | 60 | NA | 0.07 |
| | 360 | 634 | 0.17 |
| | 540 | 1110 | NA |
| | 1440 | Very High | NA |

As shown in Table 8. the concentration of dexamethasone in plasma released from the prodrugs was significantly lower than free dexamethasone administered to the rats, while the concentration of dexamethasone in the brain was comparable in both cases for up to 6 hours. The percentage of dexamethasone in the brain relative to the plasma after 6 hours was significantly higher for the prodrug versus dexamethasone alone. Thus, the data suggests that prodrugs compounds of the present invention have the potential to deliver adequate amounts of dexamethasone in the brain with less systemic exposure, thereby, reducing the toxicities of dexamethasone in the body.

While only certain features of several embodiments have been illustrated and described herein, many modifications and changes will occur to those skilled in the art. It is, therefore, to be understood that the appended claims are intended to cover all such modifications and changes as fall within the scope of the invention and the appended claims.

1. A compound having a structure of formula (I), or a pharmaceutically acceptable salt thereof, wherein formula (I) is: 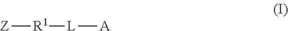
wherein Z is a therapeutic moiety;
L is a linker moiety bonded to Z via R¹, wherein R¹ is O, N, or NH, and L is selected from: 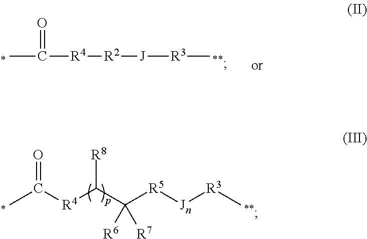
wherein * denotes the point of attachment of the linker moiety to R¹ and ** denotes the point of attachment of the linker moiety to A;
J is an aryl group or a heteroaryl group optionally substituted with one or more functional groups;
R² and R³ are independently at each occurrence a bond, a C₁-C₆ alkylene group, or a C₁-C₆ alkenylene group;
R⁴ is a bond, O or NR′;
R⁵ is a bond, a C₁-C₆ alkylene group, O or NR′, wherein R′ is independently hydrogen or a C₁-C₃ alkyl group;
R⁶, R⁷, and R⁸ are independently at each occurrence hydrogen or a C₁-C₃ alkyl group, with the proviso that at least one R⁶, R⁷, or R⁸ is a C₁-C₃ alkyl group or R⁶ and R⁷ together with a carbon to which each is attached form a C₃-C₆ cycloalkyl group;
“n” is an integer from 0 to 1, “p” is an integer from 0 to 1; and
A is an amino acid moiety or a peptide moiety.

2. The compound of claim 1, wherein A comprises a residue of one or more α-amino acids selected from the group consisting of glycine, valine, isoleucine, proline, phenylalanine, tryptophan, and combinations thereof.

3. The compound of claim 1, wherein the linker moiety is selected from: 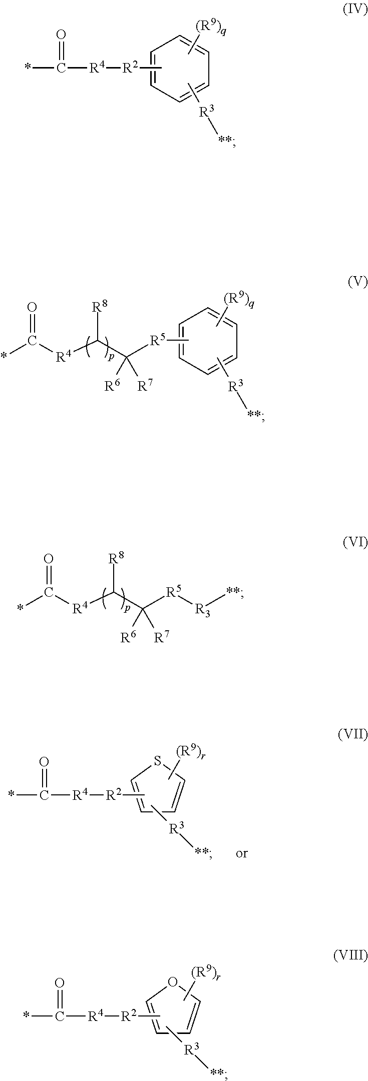
wherein “q” is an integer from 0 to 4, “r” is an integer from 0 to 2; and R⁹ is independently at each occurrence a functional group selected from alkoxy, hydroxy, halogen, amine, nitro, cyano, or dialkylamine.

4. The compound of claim 1, having a structure of formula (IX) to (XIII), or a pharmaceutically acceptable salt thereof: 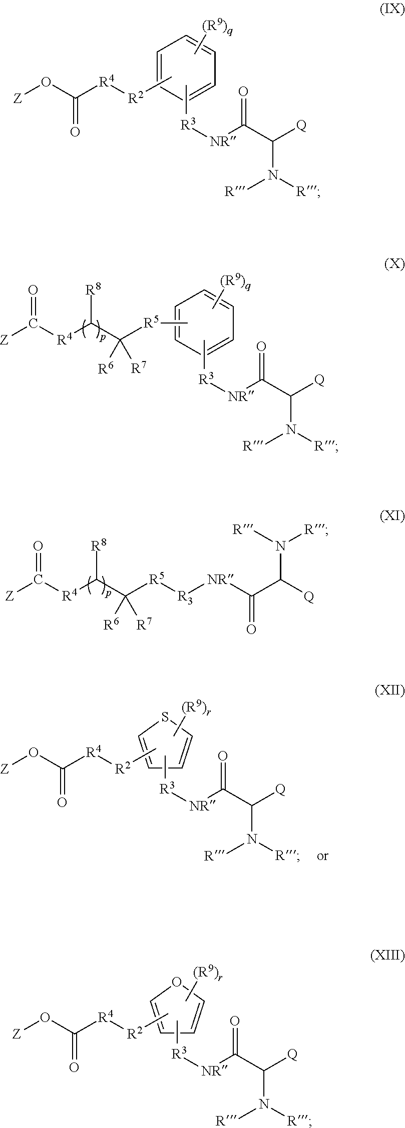
wherein “q” is an integer from 0 to 4, “r” is an integer from 0 to 2;
R⁹ is independently at each occurrence a functional group selected from alkoxy, hydroxy, halogen, amine, nitro, cyano, or dialkylamine;
R″ is hydrogen or a C₁-C₃ alkyl group; and
R′″ is independently at each occurrence hydrogen, a C₁-C₃ alkyl group, or a C(═O)CH(Q)NR′″ moiety, Q is an amino acid side chain.

5. The compound of claim 4, wherein R², R³, and R⁵ are independently a bond or a C₁-C₃ alkylene group, R⁴ is a bond, O, or NH, R⁶, and R⁷ are independently at each occurrence a methyl group, or R⁶ and R⁷ together with a carbon to which each is attached form a cyclopropyl group, and “p” is 0 or R⁸ is a C₁-C₃ alkyl group.

6. The compound of claim 4, wherein R², R³, and R⁵ are independently a bond or a C₁-C₃ alkylene group, R⁴ is a bond, R⁶ and R⁷ are independently at each occurrence a methyl group, or R⁶ and R⁷ together with a carbon to which each is attached form a cyclopropyl group, and “p” is 0 or R⁸ is a C₁-C₃ alkyl group.

7. The compound of claim 4, wherein R², R³, R⁴, and R⁵ are independently a bond, R⁶, and R⁷ are independently at each occurrence a methyl group, or R⁶ and R⁷ together with a carbon to which each is attached form a cyclopropyl group, and “p” is 0 or R⁸ is a methyl group.

8. The compound of claim 4, wherein “q” is 1-2, “r” is 1-2, and R⁹ is independently at each occurrence alkoxy, halogen, amine, or dialkylamine.

9. The compound of claim 4, wherein Q is independently at each occurrence an amino acid side chain of an amino acid selected from the group consisting of valine, proline, phenylalanine, and tryptophan.

10. The compound of claim 1, when Z is a therapeutic moiety obtained from a therapeutic agent selected from the group consisting of an antibiotic, an anti-inflammatory agent, an anti-viral agent, an anti-cancer agent, an anti-infective agent, and combinations thereof.

11. The compound of claim 1, wherein Z is a therapeutic moiety obtained from a corticosteroid.

12. The compound of claim 11, wherein the corticosteroid is selected from the group consisting of dexamethasone, prednisone, prednisolone, triamcinolone, cortisone, hydrocortisone, and betamethasone.

13. The compound of claim 4, when Z is a therapeutic moiety obtained from a therapeutic agent selected from the group consisting of an antibiotic, an anti-inflammatory agent, an anti-viral agent, an anti-cancer agent, an anti-infective agent, and combinations thereof.

14. The compound of claim 4, wherein Z is a therapeutic moiety obtained from a corticosteroid selected from the group consisting of dexamethasone, prednisone, prednisolone, triamcinolone, cortisone, hydrocortisone, and betamethasone.

15. The compound of claim 15, wherein Z is a dexamethasone residue.

16. A pharmaceutical composition comprising:
the compound of claim 1 or a pharmaceutically acceptable salt, solvate, hydrate, polymorph, or co-crystal thereof, and
a pharmaceutically carrier, diluent, or excipient.

17. A method of treating a chronic respiratory disease, an edema, or a brain disease comprising administering to a patient an effective amount of a pharmaceutical composition comprising a compound of claim 1 or a pharmaceutically acceptable salt, solvate, hydrate, polymorph, or co-crystal thereof.

18. The method of claim 17, wherein the chronic respiratory disease is chronic obstructive pulmonary disease (COPD), sarcoidosis, or asthma.

19. The method of claim 17, wherein the edema is cerebral edema, pulmonary edema, or peripheral edema.

20. The method of claim 17, wherein the brain disease is glioblastoma, medulloblastoma, glioma, or brain metastatic disease.
